(19) United States
(12) Patent Application Publication
(10) Pub. No.: US 2020/0282015 A1
(43) Pub. Date: Sep. 10, 2020

(54) TARGETED PHARMACOLOGICAL THERAPEUTICS IN UVEAL MELANOMA

(71) Applicant: Washington University, St. Louis, MO (US)

(72) Inventors: Kendall J. Blumer, St. Louis, MO (US); Michael D. Onken, St. Louis, MO (US)

(21) Appl. No.: 16/649,556

(22) PCT Filed: Sep. 21, 2018
(86) PCT No.: PCT/US2018/052299

§ 371 Date: Mar. 20, 2020

Related U.S. Application Data
(60) Provisional application No. 62/562,106, filed on Sep. 22, 2017.

(51) Int. Cl. A61K 38/15; A61K 38/12; A61K 31/395; A61P 35/00

(52) U.S. Cl. CPC A61K 38/15; A61P 35/00; A61K 31/395; A61K 38/12

(57) ABSTRACT

The present disclosure relates generally to methods and compositions for inhibiting or protecting against diseases arising from constitutively active G protein. In particular, the disclosure provides administration of FR900359, YM-254890 or a derivative thereof to down-regulate constitutively active G signaling, and is therefore useful in treatment for uveal melanoma, growth hormone-secreting pituitary tumors, tumors derived from Nevus of Ota, certain forms of other cancers (e.g. colon, lung, adenocarcinoma, skin melanoma, thyroid adenomas), cholera, Sturge-Weber Syndrome and other disorders.

### CROSS-REFERENCE TO RELATED APPLICATIONS

This application claims the benefit of U.S. Provisional Application 62/562,106, filed Sep. 22, 2017, the disclosure of which is hereby incorporated by reference in its entirety.

### FIELD OF THE INVENTION

The present disclosure relates generally to methods and compositions for treatment of diseases arising from constitutively active G protein signaling. In particular, the disclosed compositions and methods can comprise administering an FR900359, YM-254890 or a derivative thereof to a subject in need. Further, the disclosure provides administration of FR900359, YM-254890 or a derivative thereof to down-regulate constitutively active Ga signaling, therefore useful in treatment for uveal melanoma, growth hormone-secreting pituitary tumors, tumors derived from Nevus of Ota, certain forms of other cancers (e.g. colon, lung, adenocarcinoma, skin melanoma, thyroid adenomas), cholera, Sturge-Weber Syndrome and other disorders.

### BACKGROUND

A plethora of extracellular stimuli mediate cellular activity through G protein-coupled receptors, a large family of cell surface signaling molecules. The ability of G protein-coupled receptors to transduce signaling typically is induced by the binding of an appropriate ligand, resulting in a conformational change of the receptor and the subsequent interaction with the heterotrimeric αβγ guanine nucleotide-binding proteins (G proteins). G proteins located at the cytoplasmic face of the plasma membrane suffice to receive, interpret and route these signals to diverse sets of downstream target proteins. Mutations in the G protein-coupled receptors and/or G protein subunits can result in constitutively active mutants having the capacity to activate the G protein signaling cascade even in the absence of ligand. Constitutively active G protein α-subunits cause cancer, cholera, Sturge-Weber Syndrome and other disorders. While inhibitors of individual signaling pathways downstream of Gα are being studied in clinical trials, all have failed thus far. Therapeutic intervention by targeted inhibition of constitutively active Gα subunits has yet to be achieved.

Thus, there is a need for methods which allow for targeted intervention of aberrant G protein signaling. Additionally, there is a need for methods with inhibit or reduce diseases associated with constitutively active G protein signaling.

### SUMMARY

Among the various aspects of the present disclosure provide methods and pharmaceutical compositions comprising an effective amount of a constitutively active Gα subunit inhibitor, for use in treating conditions associated with constitutively active G protein signaling.

In an aspect of the disclosure provides a method of treating or preventing uveal melanoma tumor cell proliferation, the method comprising contacting uveal melanoma cells with a therapeutically effective amount of a Gα subunit inhibitor.

The present invention further provides a method of re-differentiating uveal melanoma tumor cells, the method comprising contacting uveal melanoma cells with a therapeutically effective amount of a Gα subunit inhibitor. In an aspect of the disclosure is a method of inducing polycomb repressive complex-2 mediated gene repression in uveal melanoma cells.

In a further aspect of the invention provides a method of locking a constitutively active guanine nucleotide-binding protein in a GDP-bound state. In a further aspect of the invention provides inhibiting downstream signaling pathways that result from constitutively active guanine nucleotide-binding proteins.

In certain embodiments, the Gα subunit inhibitor is selected from the group consisting of FR900359, YM-254890, a salt, prodrug or solvate thereof, an analog thereof, a derivative thereof, and any combinations thereof.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, FIG. 1C and FIG. 1D shows FR traps mutant constitutively active Gαq in the inactive GDP-bound state. (FIG. 1A-1C) depicts the effect of FR on the activity state of Gα subunits as determined by interaction with Gβγ or RGS domains in split luciferase complementation assays. FIG. 1A Effect of FR on interaction of Gβ1γ2 with the indicated wild type (WT) or mutant constitutively active (Q/L) forms of Gαq (q) or Gα13 (13). * p<0.01; ** p<0.05. FIG. 1B Potency of FR as a driver of interaction of Gβ1γ2 with wild type or mutant constitutively active Gαq. FIG. 1C Potency of FR as inhibitor of interaction of RGS2 with mutant constitutively active (Q/L) Gαq, or interaction of the RGS domain of LARG (LARG-RGS) with mutant constitutively active Gα13. FIG. 1D Potency of FR as inhibitor of signal transduction by constitutively active Gαq or Gα13 detected with an SRE(L) promoter-driven transcriptional reporter. Constitutively active Gαq (Q/L), constitutively active Gαq bearing the indicated FR binding site mutations, and constitutively Gα13 (Q/L) were studied.

FIG. 2A, FIG. 2B and FIG. 2C displays engineering an optimized FR binding site in FR-insensitive Gail is sufficient to confer FR sensitivity. FIG. 2A Amino acid residues in Gail identical to or diverged from the FR/YM binding site in Gαq are indicated in green and red, respectively. An FR/YM binding site was created in Gαi1 by introducing the eight indicated amino acid substitutions so as to match the corresponding residues of Gαq, thereby producing a chimeric Gα subunit termed Gi/q. FIG. 2B Effect of FR on guanine nucleotide exchange in vitro by Gi/q α-subunits or a mutant (R54K) corresponding to a Gαq mutant that is less sensitive to FR/YM. FIG. 2C Effect of FR on agonist-evoked signaling mediated by Gi/q. Inhibition of forskolin-induced cAMP formation by Gi-coupled cannabinoid receptors was measured in Neuro2A cells transfected with a cAMP FRET reporter and pertussis toxin (PTX)-resistant and EE epitope-tagged forms of the indicated Gα subunits, and treated with PTX to inactivate endogenously expressed Gi. Inhibition of forskolin-induced cAMP formation by a cannabinoid receptor agonist (WIN 55,212-2; WIN) was measured by FRET. Attenuation of this inhibitory effect by FR was quantified relative to vehicle controls. **p<0.05; *p<0.01.

FIG. 3A and FIG. 3B shows FR inhibits signaling by constitutively active Gαq in UM cells. Inhibition of signaling by constitutively active Gαq in UM cultured cell lines was quantified by measuring intracellular inositol 1-phosphate (IP1), a metabolically stable product of inositol 1,4,5-trisphosphate produced by Gαq-stimulated phospholipase Cβ. FIG. 3A Basal IP1 levels in UM cell lines driven by constitutively active Gαq (92.1 and Mel202) and BRAF (OCM-1A). FIG. 3B Effects of FR on IP1 levels in 92.1, Mel202 and OCM-1A cells. *p<0.01.

FIG. 4A, FIG. 4B, FIG. 4C and FIG. 4D shows FR sensitivity of growth and viability of UM cells driven by constitutively active Gαq. FIG. 4A Changes in viability of UM cells treated with FR were quantified using a water-soluble tetrazolium salt assay. The fold change in viability over time is shown for Gαq(Q209L) driven 92.1 and Mel-202 cells, and for BRAF(V600E)-driven OCM-1A cells in response to increasing concentration of FR. FIG. 4B Potency of FR as an inhibitor of UM cell viability measured as in (FIG. 4A). FIG. 4C The indicated UM cell lines were treated for 3 d with the indicated concentrations of FR and analyzed for DNA content. Flow cytometry of the Gαq(Q209L) driven cell lines (92.1 and Mel202) and BRAF(V600E)-driven OCM-treated 3 d with vehicle or FR. FIG. 4D Potency of FR as inducer of apoptosis (sub-G1 cells) and inhibitor of cell proliferation (S- and G2/M-phase cells). *p<0.01.

FIG. 5A, FIG. 5B, and FIG. 5C shows FR induces redifferentiation of UM cells driven by constitutively active Gαq. FIG. 5A Morphological changes elicited by FR. UM cell lines were treated 3 d with FR and imaged by phase contrast microscopy. FR caused UM cells driven by constitutively active Gαq (92.1 and Mel202) to lose their characteristic spindle shaped and assume a stellate shape with multiple projections. FR had no effect on BRAF-driven UM cells (OCM-1A). FIG. 5B Melanocytic differentiation of FR-treated UM cells indicated by pigmentation. UM cell lines were treated 3 d with FR. Cells were pelleted and examined macroscopically. FIG. 5C Induction of melanocytic markers by FR as indicated by immunofluorescence staining of tyrosinase (TYR), dopachrome tautomerase (DCT) and pre-melanosomal protein (PMEL) of Gαq-mutant cell lines (92.1 and Mel202) but not BRAF-driven UM cells (OCM-1A). Scale bar=50 μm.

FIG. 6A and FIG. 6B shows effects of FR on YAP-driven gene expression. FIG. 6A FR upregulates some YAP target genes but downregulates others in Gαq(Q209L)-driven 92.1 cells. Expression data from two experimental replicates are mean-centered; the color gradient corresponds to difference in log 2 ratio of expression from the mean, such that 0.5 versus −0.5 is a total difference of 2-fold in unlogged expression. FIG. 6B Potency of FR as an inducer of a YAP-driven transcriptional reporter in Gαq(Q209L)-driven Mel202 and 92.1 cells, but not in BRAF(V600E)-driven OCM-1A cells.

FIG. 7A, FIG. 7B, FIG. 7C, FIG. 7D, FIG. 7E, FIG. 7F and FIG. 7G shows FR represses expression of differentiation genes by restoring function of the polycomb repressive complex 2. Gαq-mutant 92.1 UM cells were treated with FR or vehicle, and RNA was collected 1 and 3 d after treatment for RNA-Seq analysis. FIG. 7A Unsupervised principal component analysis identified FR treatment and time in culture as the two main separable factors contributing to changes in gene expression. FIG. 7B Volcano plot comparing gene expression between FR- and vehicle-treated samples identifies a group of significantly downregulated genes (circled) associated with FR treatment. FIG. 7C Gene ontology analysis of the FR-repressed geneset shows that most are involved in developmental processes and differentiation. FIG. 7D FR-repressed genes identified as targets of the polycomb repressive complex 2 by Geneset enrichment analysis. FIG. 7E The Ezh1/2 inhibitor GSK503 blocks the morphological differentiation elicited by FR. 92.1 UM cells were treated 7 d with GSK503 and 3 d with FR and then imaged by phase contrast microscopy. FIG. 7F GSK503 decreased pigmentation of FR-treated cells, visualized by macroscopic inspection. 92.1 cells were treated 7 d with GSK503 and 3 d with FR and pelleted. FIG. 7G PRC2 inhibition by GSK503. Immunoblots of 92.1 cells treated 7 d with GSK503 show reduced histone H3 lysine 27 trimethylation.

FIG. 8A, FIG. 8B, FIG. 8C and FIG. 8D shows FR Inhibition of Gαi1 bearing an engineered FR binding site. FIG. 8A Inhibition of nucleotide exchange in vitro. Nucleotide exchange was assayed by measuring increases in fluorescence of BODIPY-GTPγS (ΔF/Fo) upon binding to the indicated purified His-tagged Gα subunits in the absence or presence of the indicated concentrations of FR. Gαi/q contains an engineered FR binding site as illustrated in FIG. 2A. Curves are representative of three independent experiments. FIG. 8B Protein expression of internally EE epitope-tagged Gα subunits in transiently transfected N2a cells as detected by immunoblotting. Gα mutants insensitive to pertussis toxin (C351G) and/or FR (R54K) are indicated. FIG. 8C Inhibition of forskolin-evoked cAMP formation by Gαi1 bearing an engineered FR binding site. Inhibition of forskolin-evoked cAMP production by an agonist (WIN 55 212-2 (WIN)) for Gαi1-coupled type 1 cannabinoid receptors in N2a cells was used to assay function of the Gαi1 subunit bearing an engineered FR binding site. N2a cells were transfected with the indicated Gα subunits and the Epac-SH187 cAMP FRET sensor, and treated or not with pertussis toxin to inactivate endogenously expressed Gαi/o. Cells then were treated as indicated with forskolin (FSK; 20 μM; black bars) for 5 min to stimulate cAMP production, and then with WIN (5 μM; grey bars) to activate endogenous Gαi/o coupled cannabinoid type 1 receptors and inhibit adenylyl cyclase. Each transfected Gα subunit bearing the C351G substitution, including those also bearing an engineered FR binding site or an FR-resistant mutation (R54K), were functional as indicated by the ability to mediate pertussis toxin-insensitive inhibition of cAMP formation. Results shown are the averages of three independent experiments. FIG. 8D FR inhibits signaling mediated by Gαi1 bearing an FR binding site. Signaling mediated by the indicated transfected forms of Gαi1 in N2a cells treated with pertussis toxin with or without the indicated concentrations of FR was assayed as described in panel C. Forskolin (FSK; black bars) and agonist (WIN; grey bars) treatment are indicated. FR concentrations are color coded as indicated. Data from one experiment representative of three independent experiments are shown.

FIG. 9A and FIG. 9B shows heatmaps of cell cycle and apoptosis gene expression in response to FR. Expression data from two experimental replicates are mean-centered, and the colorgradient corresponds to difference in log 2 ratio of expression from the mean, such that −0.5 versus 0.5 is a total difference of 2-fold in unlogged expression. Genes are ordered by fold change of FR-treated versus control from positive (left) to negative (right). FIG. 9A FR has little overall effect on expression of cell cycle genes. FIG. 9B FR evokes modest up- and down-regulation of pro- and anti-apoptotic genes.

FIG. 10A, FIG. 10B, FIG. 10C, FIG. 10D, FIG. 10E and FIG. 10F shows validation of selected FR target genes. The results for six genes showing significant changes in expression in response to FR by RNA-Seq were validated by qPCR. ADRA2A, HAND2, SCARF2, and WT1 are known targets of PRC2-directed histone methylation and all showed significant down-regulation in response to FR in Gαq(Q209L)-driven 92.1 UM cells with no effect on BRAF(B600E)-driven OCM-1A UM cells. PMP22, a known YAP target, and DCT, a pigmentation enzyme, were significantly upregulated in response to FR in Gαq(Q209L)-driven 92.1 UM cells but showed no effect on BRAF(B600E)-driven OCM-1A UM cells.

### DETAILED DESCRIPTION

The present invention provides, generally, methods and compositions for treating and preventing disorders mediated by constitutively active G-protein signaling. In particular, provided herein are compositions comprising FR900359, YM-254890 or a derivative thereof and methods of use. In particular, Applicants have surprisingly discovered that FR900359 or YM-254890 and derivatives thereof can allosterically inhibit the nucleotide exchange to trap constitutively active mutant Gαq in the inactive GDP-bound state and as such represent a therapeutic option for treatment of diseases and disorders associated with constitutively active G-protein signaling. The invention is useful, in non-limiting examples, for slowing, protecting from the effects of, or halting the progression of a condition resulting from constitutively active G-protein signaling.

The present invention also provides therapeutic compositions for treating a constitutively active G-protein modulated disease in a subject, wherein the composition comprises an inhibitor of a constitutively active G-protein and a carrier. In non-limiting examples the G-protein inhibitor is FR900359, YM-254890, a salt, prodrug or solvate thereof, an analog thereof, a derivative thereof, and any combinations thereof.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules of the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Various aspects of the invention are described in further detail in the following sections.

### (I) Compositions

One aspect of the present disclosure provides a small molecule inhibitor of a constitutively active Gα subunit. As used herein, a small molecule inhibitor of a constitutively active Gα subunit, or “Gα subunit inhibitor” includes any compound capable of downregulating, decreasing, reducing, suppressing or inactivating the amount and/or activity of a constitutively active G-protein. In some embodiments the inhibitors for use with the invention may function to inhibit constitutively active G-protein signaling, by locking the G-protein in a GDP-bound state. Compounds that decrease the activity of a constitutively active G-protein also decrease the associated downstream signaling pathways. In alternative aspects, the invention provides compositions for targeting a constitutively active G-protein, e.g., antibodies, aptamers, inhibitory peptides, inhibitory nucleic acids and the like.

A composition of the invention may optionally comprise one or more additional drug or therapeutically active agent in addition to the FR900359, YM-254890, or derivatives thereof. A composition of the invention may further comprise a pharmaceutically acceptable excipient, carrier, or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents, or antioxidants.

Other aspects of the invention are described in further detail below.

### (a) FR900359, YM-254890, and Derivatives Thereof

In general, the compounds detailed herein include compounds comprising a FR900359, structure as diagrammed below. FR900359 is a synthetic organic molecule. Its chemical elements are expressed as C₄₉H₇₅N₇O₁₅, with a molecular weight of 1002.173 g/mol. FR900359 can be isolated from the plant Ardisia crenata and its synthesis is known, for example as described by Xiong et al. Nat Chem. 2016 November; 8(11): 1035-1041, herein incorporated by reference in its entirety. FR900359 is also manufactured commercially.

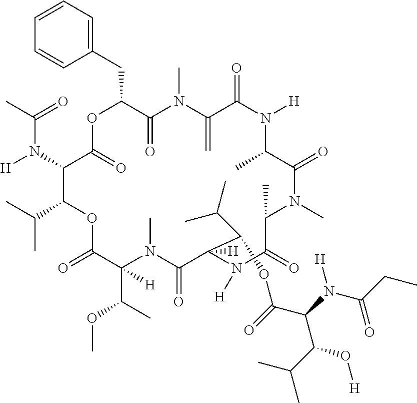 [SMILES: C=C1C(=O)N[C@@H](C)C(=O)N(C)[C@@H](C)C(=O)N[C@H]([C@@H](OC(=O)[C@@H](NC(=O)CC)[C@H](O)C(C)C)C(C)C)C(=O)N(C)[C@@H]([C@H](C)OC)C(=O)O[C@H](C(C)C)[C@H](NC(C)=O)C(=O)O[C@H](Cc2ccccc2)C(=O)N1C]

FR900359 may also be called [(1S)-1-[(3S,6R,9S,12S,18R,21S,22R)-21-acetamido-18-benzyl-3-[(1S)-1-methoxyethyl]-4,9,10,12,16-pentamethyl-15-methylidene-2,5,8,11,14,17,20-heptaoxo-22-propan-2-yl-1,19-dioxa-4,7,10,13,16-pentazacyclodocos-6-yl]-2-methylpropyl] (2S,3R)-3-hydroxy-4-methyl-2-(propanoylamino)pentanoate. In some embodiments, the disclosure provides a pharmaceutical composition comprising a therapeutically effective amount of FR900359, or a pharmaceutically acceptable salt thereof, and a pharmaceutically acceptable excipient.

Moreover, the compounds as described herein include the small molecule inhibitor is YM-254890. YM-254890 is a cyclic depsipeptide isolated from a soil bacterium Chromobacterium sp. YM-254890, has the following chemical formula:

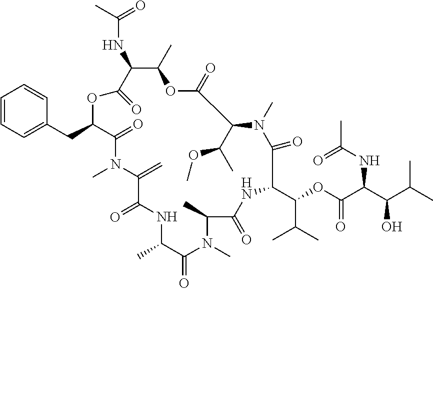

YM-254890 is also known as (1R)-1-{(3S,6S, 9S, 12S,18R,21S,22R)-21-Acetamido-18-benzyl-3-[(1R)-1-methoxyethyl]-9,10,12,16,22-pentamethyl-15-methylene-2,5,8,11,14,17,20-heptaoxo-1,19-dioxa-4,7,10,13,16-pentaazacyclodocosan-6-yl}-2-methylpropyl (2S,3R)-2-acetamido-3-hydroxy-4-methylpentanoate. YM-254890 can be synthesized, for example, as described by Xiong et al. Nat Chem. 2016 November; 8(11): 1035-1041, herein incorporated by reference in its entirety. The invention provides a pharmaceutical composition comprising an effective amount of YM-254890 or a pharmaceutically acceptable salt thereof, and a pharmaceutically acceptable excipient.

Provided herein are derivatives of FR900359 and YM-254890. FR900359 and YM-254890 derivatives are modified versions of FR900359 and YM-254890, respectively that are useful as an inhibitor of constitutively active G-protein signaling. As used herein an “FR900359 derivative” or “YM-254890 derivate” may be any derivative known in the art. FR900359 and YM-254890. FR900359 and YM-254890 derivatives are known in the art, including, for example as described in Zhang et al. Eur J Med Chem. 2018 Aug. 5; 156:847-860; Reher et al. Chem Med Chem. 2018 Aug. 20; 13(16):1634-1643; Zhang et al. Chem Med Chem. 2017 Jun. 7; 12(11):830-834; and Xiong et al. Nat Chem. 2016 November; 8(11): 1035-1041, herein incorporated by reference in their entirety.

### (b) Components of the Composition

The present disclosure also provides pharmaceutical compositions. The pharmaceutical composition comprises FR900359, YM-254890, and derivatives thereof, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to FR900359, YM-254890, and derivatives thereof. Thus, in addition to the therapies described herein, one may also provide to the subject other therapies known to be efficacious for treatment of the disease, disorder, or condition. In some embodiments, the additional drug or therapeutic agent maybe a small molecule, a polypeptide, a nucleic acid, a cell or cell lysate, a virus (e.g. oncolytic virus), and antibody or the like. In some embodiments, the administration of FR900359, YM-254890, or derivatives thereof maybe administered before or after radiation or surgery. In some embodiments, the additional drug or therapeutically active agent induces anti-inflammatory effects. In some embodiments, the secondary agent is selected from a corticosteroid, a non-steroidal anti-inflammatory drug (NSAID), an intravenous immunoglobulin, a tyrosine kinase inhibitor, a fusion protein, a monoclonal antibody directed against one or more pro-inflammatory cytokines, a chemotherapeutic agent and a combination thereof. In some embodiments, agents suitable for combination therapy include but are not limited to immune checkpoint blockades. Non-limiting examples of immune checkpoint blockade agents include inhibitors of PD-1/PD-L1, CTLA-4, IDO, TIM3, LAG3, TIGIT, BTLA, VISTA, ICOS, KIRs, CD39, Pembrolizumab, Nivolumab, Ipilimumab, Atezolizumab, Avelumab, Durvalumab. In some embodiments, the additional drug or therapeutic agent is IMCgp100. In some embodiments, the additional drug or therapeutic agent is a CYSLTR2 antagonists. In one aspect, the additional drug or therapeutically active agent is a chemotherapeutic agent. In another aspect, agents suitable for combination therapy include CAR T-cell therapy. In some embodiments, the secondary agent may be a glucocorticoid, a corticosteroid, a non-steroidal anti-inflammatory drug (NSAID), a phenolic antioxidant, an anti-proliferative drug, a tyrosine kinase inhibitor, an anti IL-5 or an IL5 receptor monoclonal antibody, an anti IL-13 or an anti IL-13 receptor monoclonal antibody, an IL-4 or an IL-4 receptor monoclonal antibody, an anti IgE monoclonal antibody, a monoclonal antibody directed against one or more pro-inflammatory cytokines, a TNF-α inhibitor, a fusion protein, a chemotherapeutic agent or a combination thereof. In some embodiments, the secondary agent is an anti-inflammatory drug. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, curcumin, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, lysofylline, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, mepolizumab, prodrugs thereof, and a combination thereof.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (d) Administration

(i) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising the FR900359, YM-254890, or derivatives thereof, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the FR900359, YM-254890, or derivatives thereof, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the the FR900359, YM-254890, or derivatives thereof may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palm itate), n-octadecanoate (stearate), n-eicosanoate (arachidate), n-docosanoate (behenate), n-tetracosanoate (lignocerate), cis-9-hexadecenoate (palm itoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3, 3, 3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the FR900359, YM-254890, or derivatives thereof, may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of FR900359, YM-254890, or derivatives thereof, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of the FR900359, YM-254890, or derivatives thereof may be encapsulated in a microemulsion by any method generally known in the art.

In yet another embodiment, the FR900359, YM-254890, or derivatives thereof, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

Generally, a safe and effective amount FR900359, YM-254890, or derivatives thereof is, for example, that amount that would cause the desired therapeutic effect in a subject while minimizing undesired side effects. In various embodiments, an effective amount of an FR900359, YM-254890, or derivatives thereof described herein can substantially inhibit constitutively active G-protein signaling and treat associated diseases. In some embodiments, an effective amount is an amount capable of allosterically inhibiting the nucleotide exchange to trap constitutively active mutant Gαq in the inactive GDP-bound state and as such represent a therapeutic option for treatment of diseases and disorders associated with constitutively active G-protein signaling.

When used in the treatments described herein, a therapeutically effective amount of FR900359, YM-254890, or derivatives thereof can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate diseases and disorders associated with constitutively active G-protein signaling.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of FR900359, YM-254890, or derivatives thereof can occur as a single event or over a time course of treatment. For example, FR900359, YM-254890, or derivatives thereof can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease or disorder associated with constitutively active G-protein signaling.

The present disclosure encompasses pharmaceutical compositions comprising a G-protein inhibitor as disclosed above, so as to facilitate administration and promote stability of the active agent. For example, a G-protein inhibitor of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the invention, the subject may be a human or any other animal. In particular embodiments, the subject is selected from the group consisting of primate, equine, ovine, caprine, leporine, avian, feline, rodent, or canine. Methods of preparing and administering constitutively active G-protein inhibitor disclosed herein to a subject in need thereof are well known to or are readily determined by those skilled in the art. The route of administration of a constitutively active G-protein inhibitor can be, for example, peripheral, oral, parenteral, by inhalation or topical.

### (II) Methods

The present disclosure encompasses a method of treating a disease or disorder associated with constitutively active G-protein signaling. Aberrant G-protein signaling pathways are involved in many diseases such as, in non-limiting examples, uveal melanoma, growth hormone-secreting pituitary tumors, tumors derived from Nevus of Ota, certain forms of other cancers (e.g. colon, lung, adenocarcinoma, skin melanoma, thyroid adenomas), cholera, and Sturge-Weber Syndrome. Generally, the method comprises administration of a therapeutically effective amount of FR900359, YM-254890, or derivatives thereof, so as to down-regulate constitutively active Gα signaling, lock a G-protein in a GDP bound state, inhibit proliferation of cancer cells, re-differentiate cancer cells, increase polycomb repressive complex-2 (PRC2) activity in cancer cells, inhibit a constitutively active Gα associated disease, slow the progress of a constitutively active Gα associated disease or limit the development of a constitutively active Gα associated disease. In some embodiments the cancer cells are Uveal Melanoma (UM) cells. In another aspect, the present disclosure encompasses a method suppressing constitutively active Gα signaling in a subject in need thereof or in a biological sample, the method comprising administering to the subject, or contacting the biological sample with a composition comprising a therapeutically effective amount of FR900359, YM-254890, derivatives thereof or combinations thereof. In yet another aspect, the present disclosure encompasses of inhibiting tumor growth in a subject in need thereof, the method comprising administering to the subject a composition comprising a therapeutically effective amount a compound FR900359, YM-254890, derivatives thereof or combinations thereof. In still yet another aspect, the present disclosure provides a composition comprising FR900359, YM-254890, derivatives thereof or combinations thereof, for use in vitro, in vivo, in situ or ex vivo. Suitable compositions comprising FR900359, YM-254890, or derivatives thereof are disclosed herein, for instance those described in Section I.

According to an aspect of the invention a pharmaceutical composition comprising FR900359, YM-254890, derivatives thereof or combinations thereof can treat, reduce, or prevent a disease, disorder, or condition associated with constitutively active Gα signaling. As used herein the term “treating” refers to: (i) preventing a disease, disorder or condition from occurring in an animal or human that may be predisposed to the disease, disorder and/or condition but has yet been diagnosed as having it; (ii) inhibiting the disease, disorder, or condition, i.e., arresting its development or progression; and/or (iii) relieving the disease, disorder or condition, i.e., causing regression, remission of the disease, disorder and/or condition. For example, with respect to uveal melanoma, treatment may be measure by quantitatively or qualitatively to determine the presence of absence of the disease, or its progression or regression using for example symptoms associated with the diseases or clinical indication associated with the pathology.

G protein-coupled receptors (GPCRs) are integral membrane proteins that comprise one of the largest classes of proteins in the human genome. G proteins are key mediators of G protein-coupled receptor signaling, which facilitates a plethora of important physiological processes. Agonist binding to a GPCR stabilizes an active conformation of the receptor, which activates intracellular heterotrimeric guanine nucleotide binding proteins (G proteins). G proteins are composed of α, β and γ subunits, which on activation dissociate from GPCRs and modulate a range of intracellular effectors. Constitutively active G protein a subunits cause cancer, cholera, Sturge-Weber syndrome, and other disorders. Therapeutic intervention by targeted inhibition of constitutively active Gα subunits in these disorders has yet to be achieved. Applicants have shown that constitutively active Gαq in uveal melanoma (UM) cells is inhibited by the cyclic depsipeptide FR900359 (FR), YM-254890 (YM), and derivatives thereof. Specifically, the compositions of the disclosure allosterically inhibited guanosine diphosphate-for-guanosine triphosphate (GDP/GTP) exchange to trap constitutively active Gαq in inactive, GDP-bound Gαβγ heterotrimers. Allosteric inhibition of other Gα subunits was achieved by the introduction of an inhibitor-binding site. In UM cells driven by constitutively active Gαq, the compositions as disclosed herein inhibited second messenger signaling, arrested cell proliferation, reinstated melanocytic differentiation, and stimulated apoptosis. FR, YM and derivatives thereof promoted UM cell differentiation by reactivating polycomb repressive complex 2 (PRC2)-mediated gene silencing, a heretofore unrecognized effector system of constitutively active Gαq in UM. Constitutively active Gαq and PRC2 therefore provide therapeutic targets for diseases and disorders mediated by constitutively active Gα.

In an aspect the disclosure provides a method of re-differentiating uveal melanoma tumor cells, the method comprising contacting uveal melanoma cells with a therapeutically effective amount of FR900359, YM-254890, or derivatives thereof. As used herein, “re-differentiating” means a process by which one or more dedifferentiated cells (e.g. uveal melanoma cells) return to their original specialized form. In particular re-differentiation may include, without being limited, morphological changes indicative of re-differentiation, increased melanocytic pigmentation, and increased expression of differentiation specific gene products. In some embodiments, the disclosure provides a method of inducing polycomb repressive complex-2 mediated gene repression in uveal melanoma cells. It was surprisingly discovered that by inhibiting constitutively active Gα in uveal melanoma results in reversing the repression of gene sets that are targets of epigenetic silencing by the polycomb repressive complex 2 (PRC2), and control differentiation and development. In some embodiments, a method of re-differentiating cells includes repressing ADRA2A (alpha-adrenergic receptor-2A) and/or HAND2 (heart and neural crest derivatives expressed-2) expression or activity, comprising administering an effective amount or FR900359, YM-254890, or derivatives thereof.

In another aspect, the disclosure provides a method of locking a constitutively active guanine nucleotide-binding protein in a GDP-bound state. The method comprises administering an effective amount of FR900359, YM-254890, or derivatives thereof. In some embodiments, the method includes inhibiting downstream signaling pathways that result from constitutively active guanine nucleotide-binding proteins. In non-limiting examples, such pathways include the modulation of YAP, adenylyl cyclase, phospholipase C, the mitogen activated protein kinases (MAPKs), extracellular signal regulated kinase (ERK) c-Jun-NH2-terminal kinase (JNK) and p38 MAPK.

In each of the above embodiments, the Gα subunit inhibitor may be selected from the group consisting of FR900359, YM-254890, a salt, prodrug or solvate thereof, an analog thereof, a derivative thereof, and any combinations thereof. The compounds according to the disclosure are effective over a wide dosage range. The exact dosage will depend upon the route of administration, the form in which the compound is administered, the subject to be treated, the body weight of the subject to be treated. Pharmaceutical compositions suitable for use in the present disclosure include compositions wherein the active ingredients are contained in a therapeutically effective amount to achieve its intended purpose. Determination of the effective amounts is well within the capability of those skilled in the art.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery. In a particular embodiment, the pharmaceutical composition is formulated for inhalation or oral administration.

Methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### (III) KITS

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to compositions and pharmaceutical formulations comprising a constitutively active G-protein modulation agent, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As various changes could be made in the above-described reporter molecules and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: FR Traps Constitutively Active Gαq in the GDP-Bound State

To determine whether constitutively active Gαq undergoes appreciable GDP/GTP exchange in UM cells a common oncogenic GTPase-defective mutant Gαq(Q209L) was evaluated for the ability to be trapped in the GDP-bound state by FR. The GDP- and GTP-bound states of Gαq(Q209L) were assessed in living cells by detecting interaction with Gβγ subunits, which bind preferentially to GDP-loaded Gα subunits (1), or with RGS2, which binds GTP- but not GDP-loaded Gαq (22). To detect protein-protein interactions, a split luciferase complementation assays was used (23), as employed to study activation state-dependent interaction between Gα subunits and cognate binding partners (24).

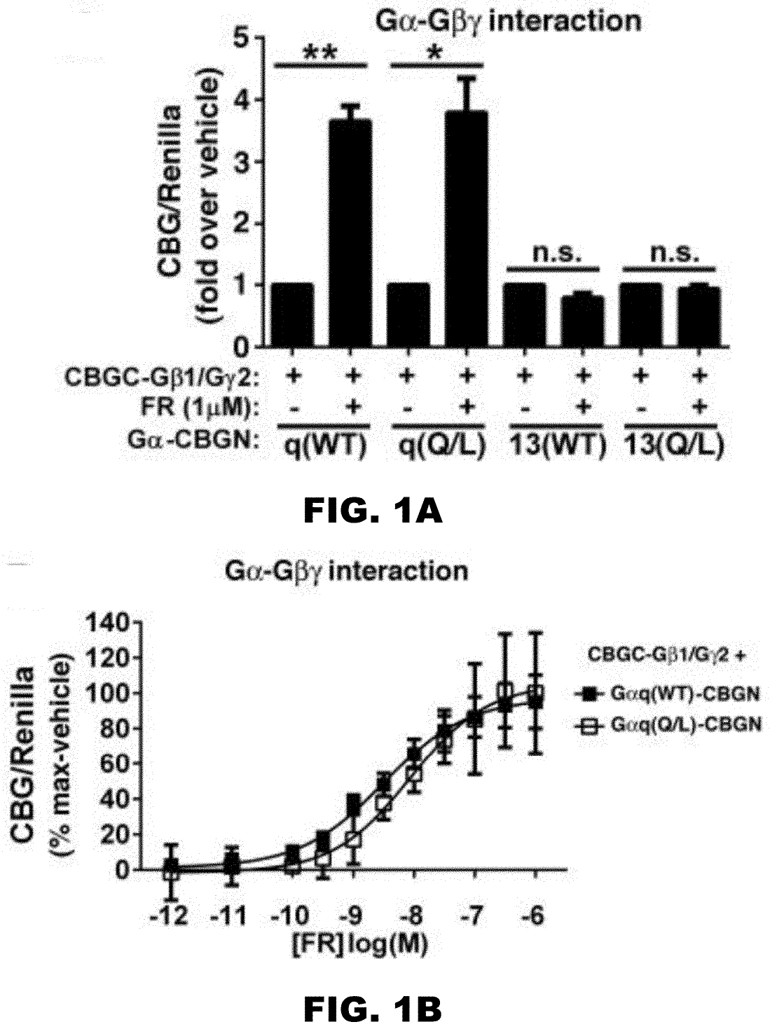

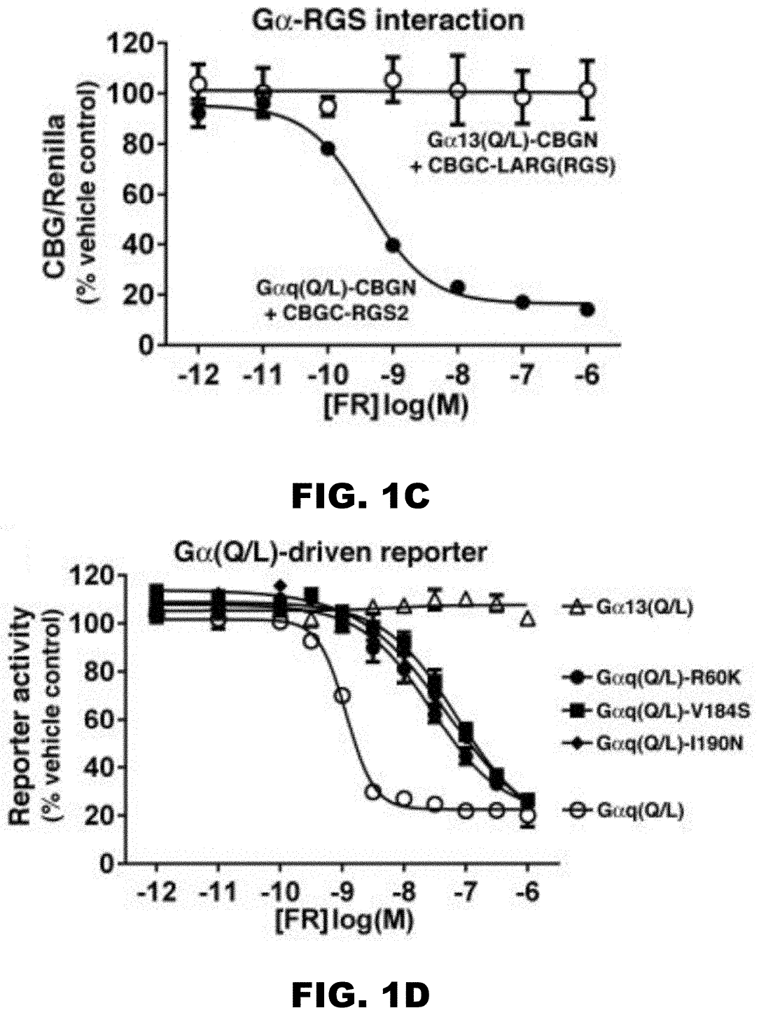

Results indicated that FR is able to trap constitutively active Gαq in the GDP-bound state. FR increased the level of split luciferase complementation for wild type or constitutively active Gαq interacting with Gβ1γ2 (FIG. 1B; EC₅₀ 3 nM and 9 nM, respectively). Conversely, FR drove constitutively active Gαq out of the active GTP-bound state, as indicated by inhibition of interaction between constitutively active Gαq and RGS2 (FIG. 1C; IC₅₀ 0.4 nM). FR was highly selective for Gαq, revealed by its lack of effect on the interaction of wild type or constitutively active Gα13 with Gβ1γ2 (FIG. 1A), or constitutively active Gα13 with the RGS domain of LARG (FIG. 1C). Thus, FR drives constitutively active Gαq from its active GTP-bound state into inactive GDP-bound Gαβγ complexes.

As a further test of the ability of FR to inhibit constitutively active Gαq, downstream signaling was measured. FR inhibited induction of a transcriptional reporter driven by constitutively active Gαq (FIG. 1D; IC50 1 nM), but FR had no effect on expression of the reporter when driven by constitutively active Gα13. Three amino acids involved in the FR binding site were chosen from crystallographic and mutagenesis studies using wild type Gαq and an inhibitor nearly identical to FR, YM-254890 (YM) (19). Single amino-acid substitutions of each (R60K, V184S, or 1190N) in constitutively active Gαq blunted the inhibitory potency of FR (FIG. 1D; IC₅₀ 30 to 70 nM), demonstrating that FR targets constitutively active and wild type Gαq by the same mechanism.

An important discovery is that GDP/GTP exchange is an underappreciated vulnerability of constitutively active G protein α-subunits, and one that can be exploited pharmacologically in UM and other diseases. Although Gα subunits undergo GDP/GTP exchange slowly in vitro, the inventors have discovered that exchange occurs in cells at rates sufficient for constitutively active Gαq to be trapped in the inactive GDP-bound state when cells are treated with FR, an allosteric inhibitor of GDP release. When trapped by FR, GDP-bound constitutively active Gαq assembles into Gαβγ heterotrimers, further suppressing GDP release and stabilizing the inactive state. Because FR-bound Gq heterotrimers are refractory to activation by GPCRs (20), signaling networks downstream of constitutively active Gαq are attenuated.

As the above work involved constitutively active Gαq in UM, it is anticipated that constitutively active forms of Gα subunit subtypes that drive other types of cancers may also be vulnerable to allosteric inhibitors of GDP release. Constitutively active Gα11 in UM (12) and Gα14 in vascular tumors (30) should be susceptible because wild type forms of these Gα subunits are sensitive to FR (20). Although other subtypes of Gα subunits are not sensitive to FR, all Gα subunits possess a diverged but related form of the allosteric regulatory site in Gαq that binds FR. This site includes conserved features of linker 1, which stabilizes the GDP-bound state by interacting with helix 1, helix A, and helix F as part of the universal mechanism that regulates GDP release.

### Example 2: FR Inhibits Gαi1 with an Engineered FR Binding Site

FR inhibits receptor-evoked signaling by wild type Gαq and its close relatives Gα11 and Gα14, but not other Gα subunits (20). Whether FR-insensitive Gα subunits could be converted into FR-sensitive forms by the introduction of an FR binding site was tested. It was reasoned that this might be possible because all Gα subunits release GDP by a common allosteric mechanism (25) and because structural elements of the allosteric relay include the FR binding site (19, 25). Moreover, FR-insensitive Gα subunits do contain a similar but diverged form of the FR binding site.

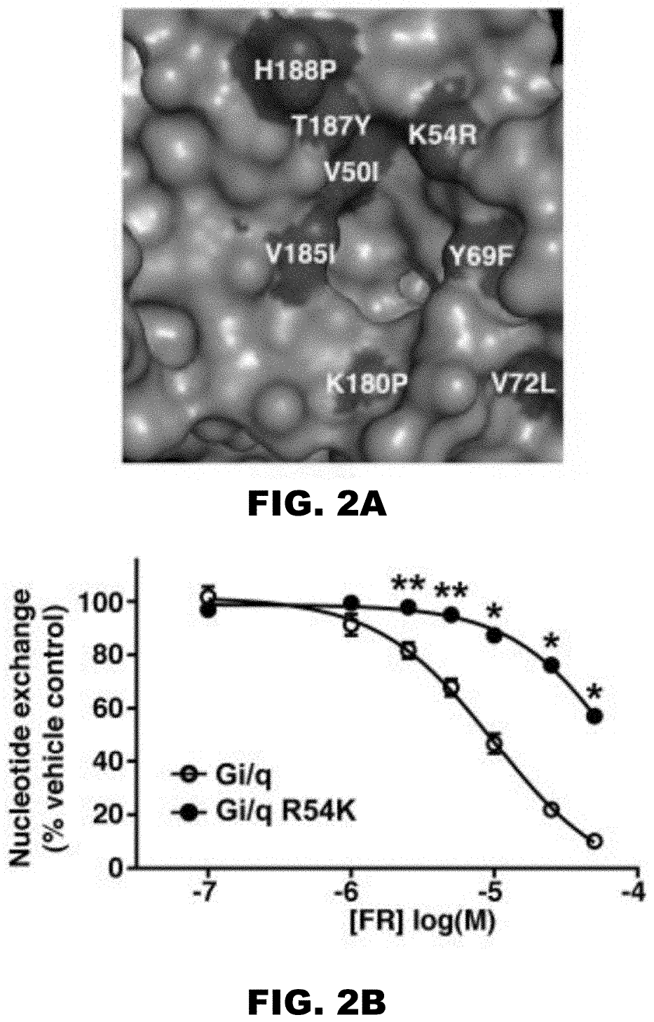

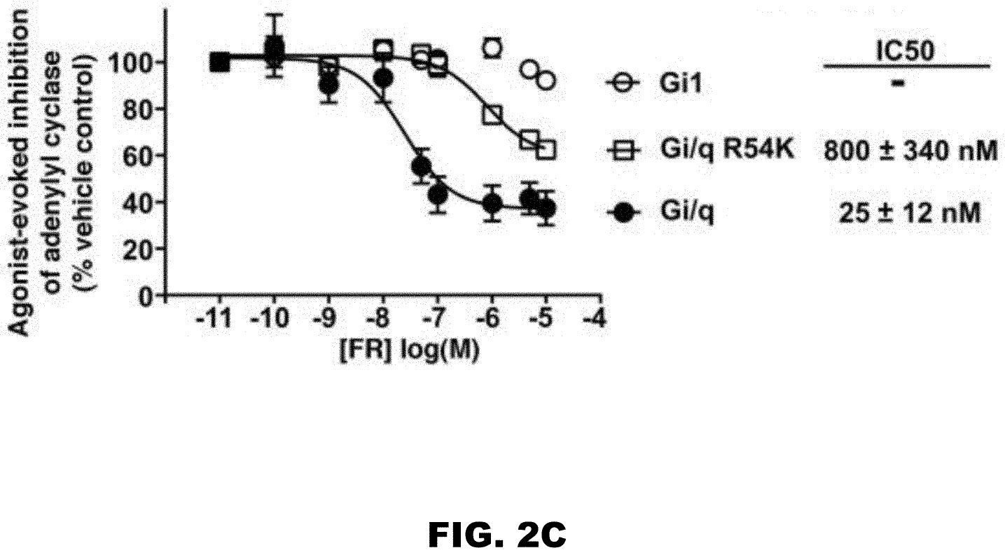

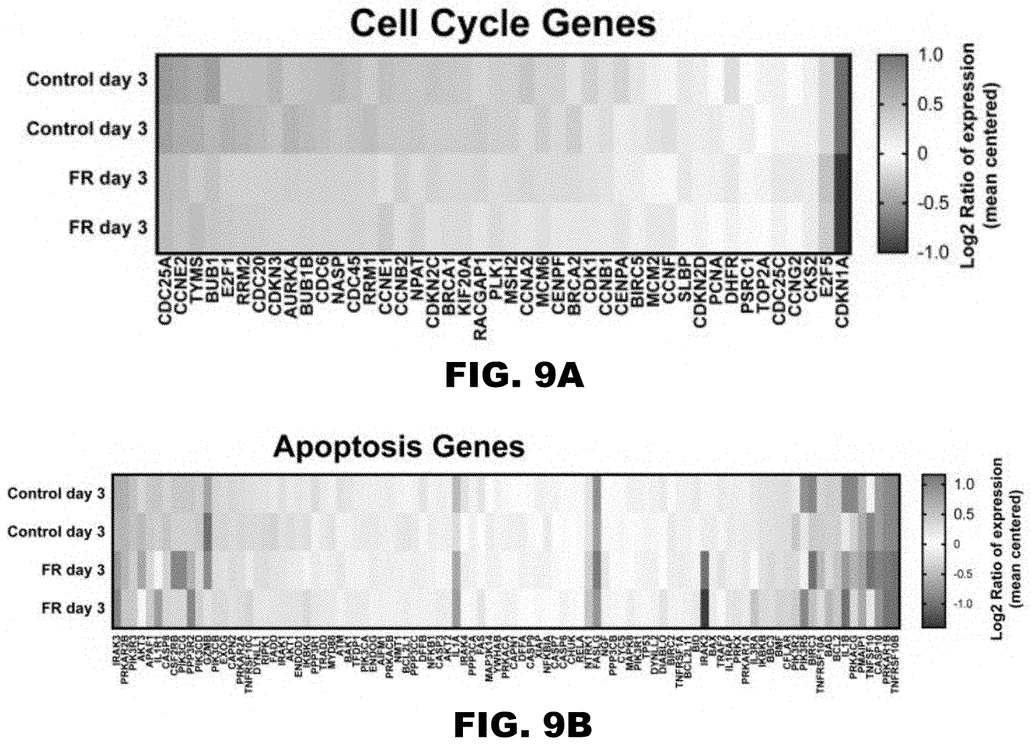

To test this, eight diverged amino acids in Gαi1 to match their counterparts in the FR/YM binding site of Gαq, producing “Gαi/q” (illustrated in FIG. 2A). Gαi1 was chosen because it is insensitive to FR (20). A Gαi/q(R54K) mutant also was made, which corresponds to an amino acid substitution in Gαq that blunts inhibition by YM (19). FR inhibited in vitro nucleotide exchange by Gαi/q (FIG. 2B). As expected, FR was ˜10-fold less potent toward Gαi/q(R54K). In addition, FR was able to inhibit signaling downstream of Gαi/q. FR attenuated the ability of signaling from the cannabinoid type 1 receptor via Gαi/q to inhibit forskolin-induced cAMP accumulation (IC₅₀=25 nM; FIG. 2C and FIG. 9). FR was ˜30-fold less potent (IC₅₀˜800 nM; FIG. 2C) toward Gαi/q(R54K). Thus, FR appears to inhibit Gαi/q and wild-type Gαq by similar mechanisms. This finding suggests that FR-like molecules may be able to target the analogous, but distinct, binding sites of other subtypes of Gα subunits, providing a general approach to discover novel chemical probes of Gα function and potential therapeutics for various diseases. In addition, this approach may be efficacious for diseases that are driven by multiple GPCRs, for which blocking a single receptor is ineffective.

### Example 3: FR Inhibits Signaling by Constitutively Active Gαq in UM Cells

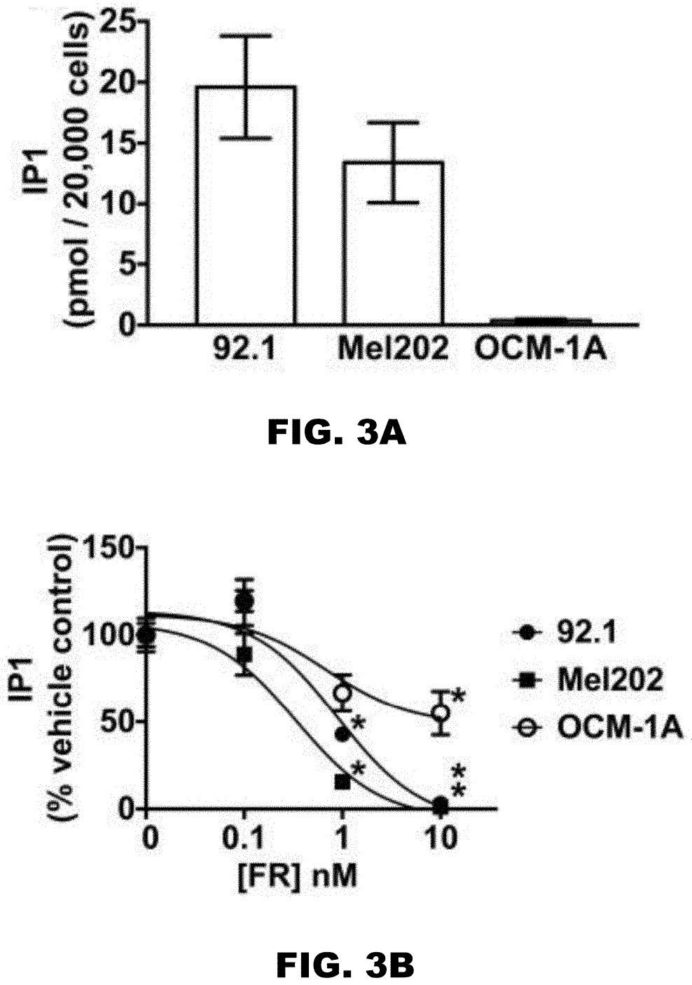

To determine whether FR inhibits signal transduction by constitutively active Gαq in UM cells, two UM cell lines (Mel202 and 92.1) driven by constitutively active Gαq(Q209L) were analyzed. A third UM cell line (OCM-1A) driven by constitutively active BRAF(V600E) served as a negative control. Signaling by Gαq-stimulated phospholipase Cβ was quantified based on levels of inositol monophosphate (IP1), a metabolically stable product of inositol 1,4,5-trisphosphate (IP3) produced by cleavage of phosphatidylinositol 4,5-bisphosphate (PIP2). In the absence of FR, IP1 levels (FIG. 3A) were >50-fold higher in Gαq(Q209L)-driven Mel202 and 92.1 cells relative to BRAF(V600E)-driven OCM-1A cells. FR reduced IP1 levels in Mel202 and 92.1 cells>50-fold (FIG. 3B), but had only modest effect (˜2-fold) on OCM-1A cells (FIG. 3B). Thus, FR strikingly inhibited second messenger production driven by constitutively active Gαq in UM cells.

### Example 4: FR Inhibits UM Tumor Cell Proliferation and Survival

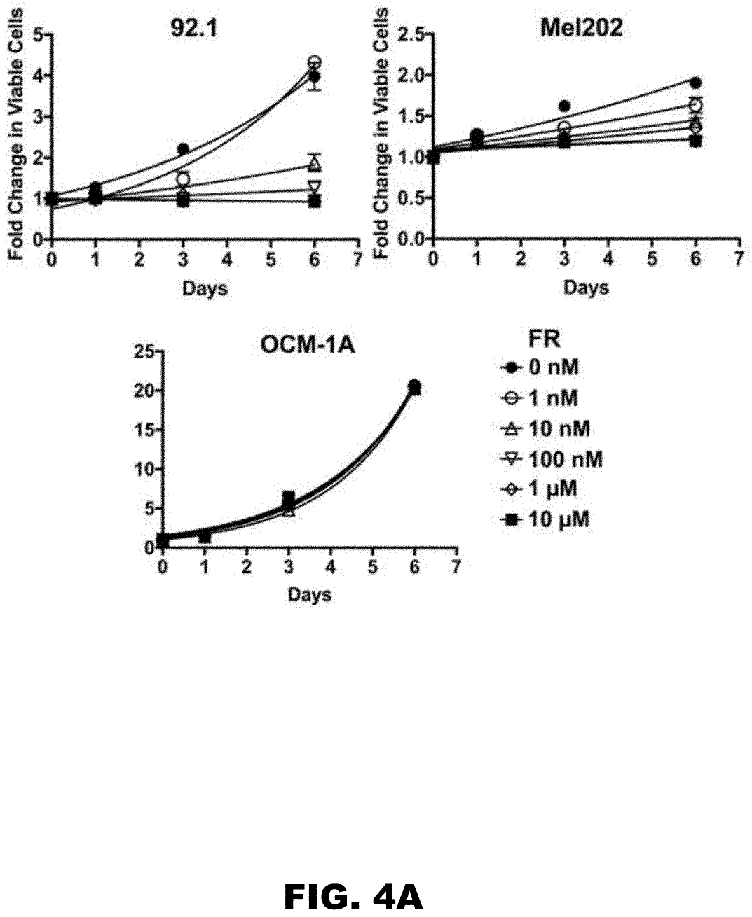

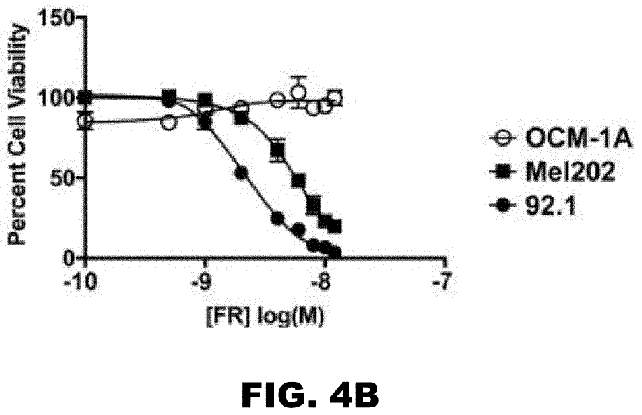

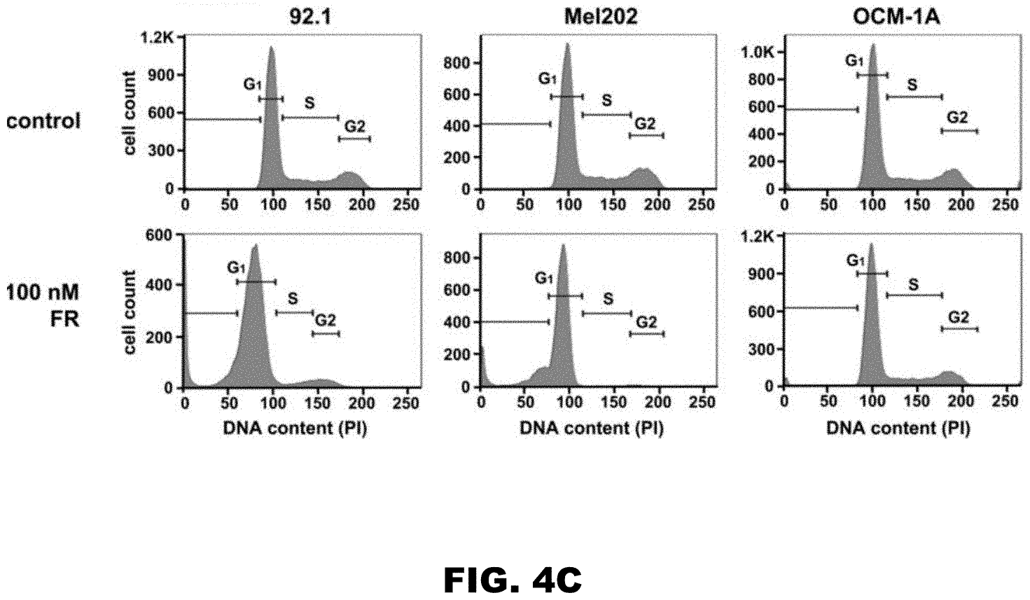

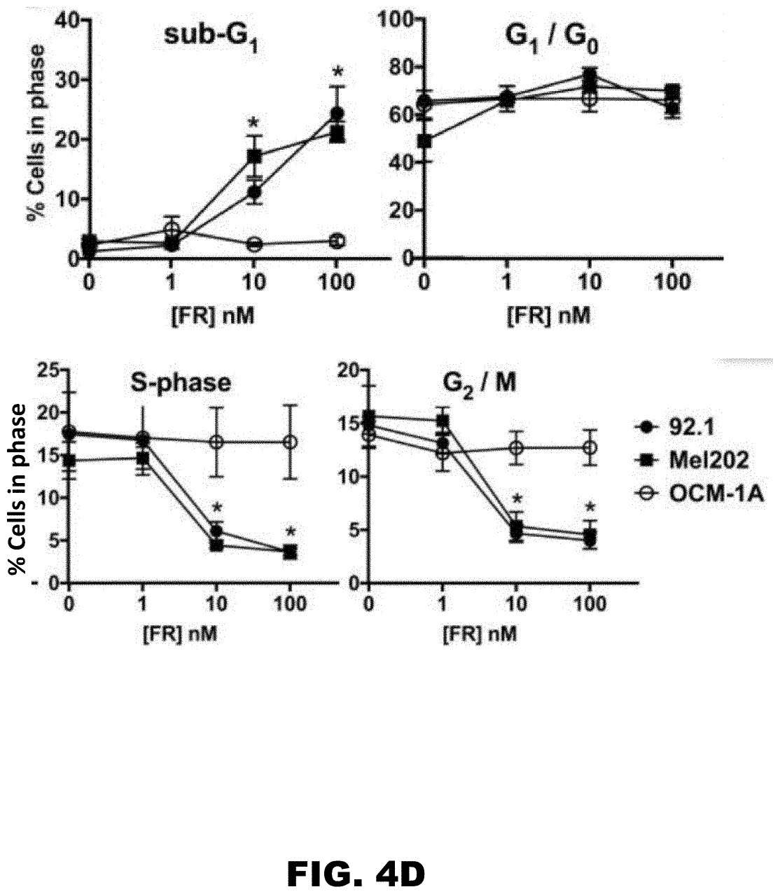

During the preceding experiments, it was observed that FR treatment decreased viability of Gαq(Q209L)-driven UM tumor cells. Quantification confirmed that FR inhibited proliferation of Gαq(Q209L)-driven Mel202 and 92.1 UM cells (FIG. 4A-B; EC₅₀ 6 nM and 2 nM, respectively), with no effect on proliferation of BRAF(V600E)-driven OCM-1A cells even at high concentration (10 μM). Flow cytometry revealed that FR induced cell cycle inhibition (decreased levels of S- and G2/M-phase cells) and apoptosis (increased levels of sub-G1/G0 cells) for Mel202 and 92.1 cells (FIG. 4C-D), with no effect on OCM-1A cells (FIG. 4C-D). FR therefore inhibited proliferation and survival in UM cells that had constitutively active Gαq as their oncogenic driver.

### Example 5: FR Promotes Melanocytic Re-Differentiation of UM Cell Lines

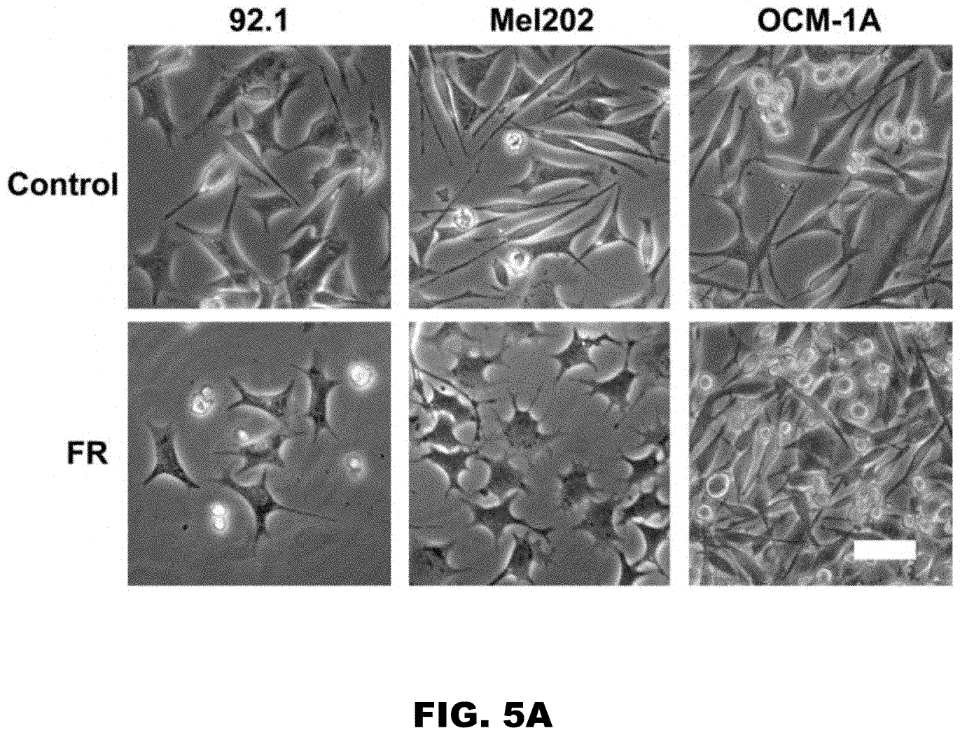

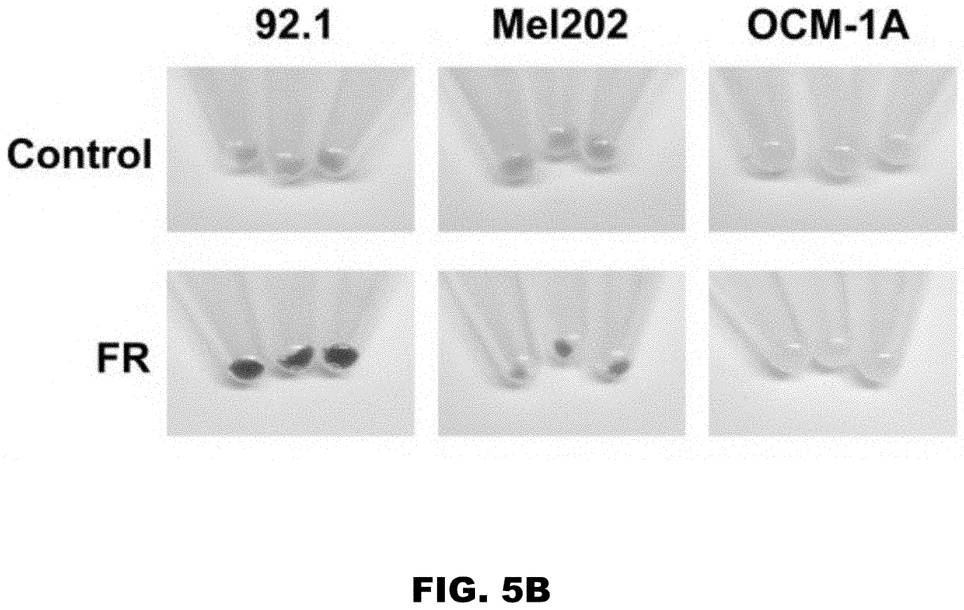

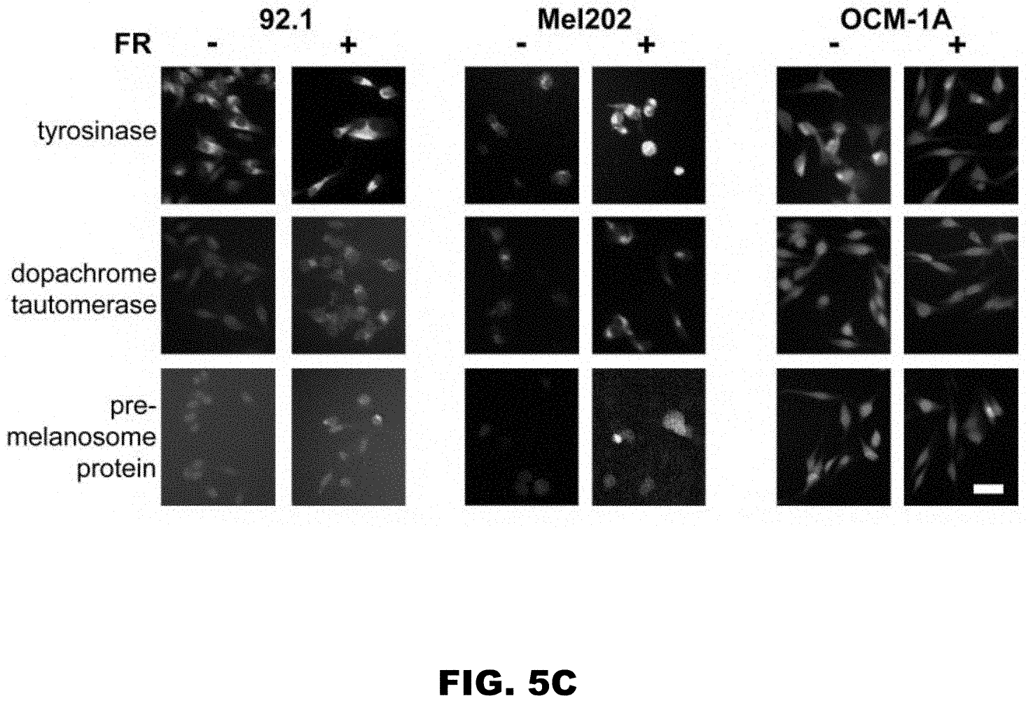

FR treatment caused Gαq(Q209L)-driven Mel202 and 92.1 cells to undergo morphological changes indicative of re-differentiation. These FR-treated cells lost spindle morphology, became flatter, and produced multiple projections (FIG. 5A), when compared with vehicle-treated cells or FR-treated OCM-1A cells. FR strikingly increased melanocytic pigmentation in Mel202 and 92.1 cells, compared with vehicle-treated cells or FR-treated OCM-1A cells (FIG. 5B). FR increased the expression levels of two pigmentation enzymes (tyrosinase (TYR) and dopachrome tautomerase (DCT)) and a melanosome structural protein (PMEL) in Mel202 and 92.1 cells but not in OCM-1A cells (FIG. 5C). Thus, FR antagonized the de-differentiation process driven by constitutively active Gαq in UM cells.

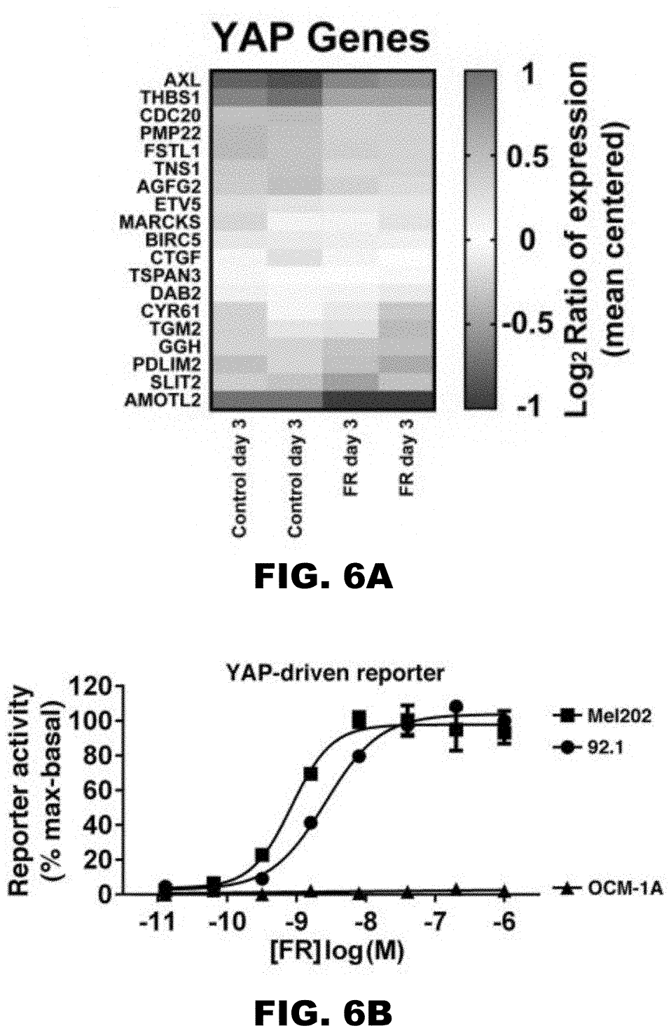

To explore how FR regulates UM cell phenotypes, global gene expression by RNA-Seq was performed. First, the inventors focused on YAP-regulated genes because the YAP protein is activated downstream of constitutively active Gαq in UM cell lines (26, 27). As expected, certain YAP target genes were downregulated by FR; however, others were upregulated (FIG. 6A). Moreover, FR caused increases in the expression of a YAP-driven transcriptional reporter in Mel202 and 92.1 cells (EC50 0.8 and 3 nM, respectively; FIG. 6B), but FR had no effect in BRAF(V600E)-driven OCM-1A cells (FIG. 6B). Therefore, rather than simply activating YAP to induce target gene expression, constitutively active Gαq exerts gene-specific effects on YAP-mediated transcription.

FR caused an apoptotic response in UM cells, as described above. However, FR treatment did not lead to striking upregulation of pro-apoptotic genes or to downregulation of survival genes. Instead, two pro-apoptotic members of the BCL2 family were downregulated modestly in response to FR—BBC3/PUMA decreased 3.5-fold (p<0.001) and PMAIP1/NOXA decreased 2.5-fold (p<0.001) (FIG. 9). No other BCL2 family members showed significant change (p<0.05, fold change>2), and broader examination of apoptosis-related genes showed only small effects (FIG. 9). These results are consistent with evidence that intrinsic and extrinsic apoptotic pathways function independently of gene transcription (28, 29).

Cell-cycle genes were also relatively unaffected by FR, as indicated by gene set enrichment analysis and direct comparison of the RNA-Seq data (FIG. 9 and Table 1). The cyclin-dependent kinase inhibitor p21CIP1 (CDKN1A, down 3.0 fold, p<0.001) was downregulated, and several cell cycle genes showed upward trends lacking statistical significance (p>0.05, fold change<2) (FIG. 9). Targets of E2F, a transcription factor positively regulated by cyclin-dependent kinases, were positively enriched (Table 1). These results suggest that the anti-proliferative effects of FR are not mediated primarily by short-term changes in cell cycle gene expression.

TABLE 1
Gene Set Enrichment Positive Correlation

| | | | | NOM | [symbol] | FWER | RANK |
|---|---|---|---|---|---|---|---|
| NAME | SIZE | ES | NES | p-val | q-val | p-val | AT MAX |
| DUTERIRE_ESTRADIOL_RESPONSE_24HR_UP | 264 | 0.6517718 | 3.44795 | 0 | 0 | 0 | 1877 |
| XOBAYASHI_EGFR_SIGNALING_24HR_ON | 209 | 0.662429 | 3.421171 | 0 | 0 | 0 | 2070 |
| ZHANG_TIX_TARGETS_60HR_ON | 230 | 0.6492758 | 3.392733 | 0 | 0 | 0 | 2050 |
| HOSTY_CERVICAL_CANCER_PROLIFERATION_CLUSTER | 120 | 0.7079712 | 3.349099 | 0 | 0 | 0 | 1252 |
| FOURNIER_AGNAR_DEVELOPMENT_LATE_2 | 222 | 0.6335315 | 3.348332 | 0 | 0 | 0 | 2283 |
| SOTHRIOU_BREAST_CANCER_GRADE_1_VS_3_UP | 124 | 0.6922286 | 3.341391 | 0 | 0 | 0 | 1433 |
| WINNEPENNINCKK_MELANOMA_METASTASIS_UP | 128 | 0.6813596 | 3.238367 | 0 | 0 | 0 | 2248 |
| KROONQUIST_IL6_DEPRIVATION_UN | 81 | 0.7235784 | 3.209366 | 0 | 0 | 0 | 1761 |
| FERREIRA_EWINGS_SARCOMA_UNSTABLE_VS_STABLE_UP | 165 | 0.5753432 | 3.206096 | 0 | 0 | 0 | 2326 |
| MITSIAGES_RESPONSE_TO_APLIOIN_DN | 210 | 0.622916 | 3.206297 | 0 | 0 | 0 | 2103 |
| PENG_LEUCINE_DEPRIVATION_DN | 152 | 0.6417515 | 3.204675 | 0 | 0 | 0 | 2063 |
| SHEDOIN_LUNG_CANCER_POOR_SURVIVAL_A6 | 338 | 0.5789082 | 3.164519 | 0 | 0 | 0 | 2483 |
| ZHANG_TLX_TARGETS_DN | 77 | 0.7067005 | 3.159272 | 0 | 0 | 0 | 2050 |
| GOBERT_OLIGODENDROCYTE_DIFFERENTIATION_UP | 430 | 0.5668274 | 3.154917 | 0 | 0 | 0 | 2244 |
| CHANG_CYCLING_GENES | 119 | 0.6553933 | 5.149013 | 0 | 0 | 0 | 1980 |
| KANG_DOXORUBICIN_RESISTANCE_UP | 50 | 0.7535572 | 3.106621 | 0 | 0 | 0 | 1422 |
| BEN_INTESTINE_PROBIOTICS_24HR_UP | 367 | 0.5622381 | 3.105234 | 0 | 0 | 0 | 2857 |
| SARRIO_EPITHELIAL_MESENCHYMAL_TRANSITION_UP | 134 | 0.6312098 | 3.090752 | 0 | 0 | 0 | 2243 |
| WHITEFORD_PEDIATRIC_CANCER_MARKERS | 94 | 0.57131 | 3.080914 | 0 | 0 | 0 | 1423 |
| PUHANA_BBCA2_PCC_NETWORK | 294 | 0.57710908 | 3.074268 | 0 | 0 | 0 | 2066 |
| MARSON_BOUND_BY_E2F4_UNSTIMULATED | 459 | 0.5462862 | 3.072465 | 0 | 0 | 0 | 2244 |
| BASAM_VUK1_TARGETS_UP | 205 | 0.5920223 | 3.06783 | 0 | 0 | 0 | 1873 |
| BLUM_RESPONSE_TO_SAURASIB_DN | 265 | 0.5754255 | 3.063616 | 0 | 0 | 0 | 2066 |
| ZHANG_TLX_TARGETS_36HR_DN | 143 | 0.5208622 | 3.060966 | 0 | 0 | 0 | 2108 |
| REACTOME_CELL_CYCLE_MITOTIC | 222 | 0.580772 | 3.036345 | 0 | 0 | 0 | 2316 |
| BURTON_ADIPOGENESIS_3 | 84 | 0.6783643 | 3.031054 | 0 | 0 | 0 | 2050 |
| FUJI_VBX1_TARGETS_DN | 155 | 0.6078871 | 3.02593 | 0 | 0 | 0 | 2541 |
| ODONNELL_TFRC_TARGETS_DN | 80 | 0.6865559 | 3.017553 | 0 | 0 | 0 | 1976 |
| CROONQUIST_NRAS_SIGNALING_DN | 63 | 0.7114243 | 3.016316 | 0 | 0 | 0 | 1243 |
| GRAHAM_CML_DIVIDING_VS_NORMAL_QUIESCENT_UP | 153 | 0.6183688 | 3.014998 | 0 | 0 | 0 | 2263 |
| ZHOU_CELL_CYCLE_GENES_IN_IR_RESPONSE_24HR | 99 | 0.6436814 | 3.009834 | 0 | 0 | 0 | 2244 |
| [symbol] _METASTASIS_UP | 159 | 0.5994755 | 2.989941 | 0 | 0 | 0 | 2283 |
| HOFFMANN_LARGE_TO_SMALL_RE_BR_LYMPHOCYTE_UP | 128 | 0.6149781 | 2.98444 | 0 | 0 | 0 | 2244 |
| ZHAN_MULTIPLE_MYELOMA_PR_UP | 40 | 0.7560601 | 2.977059 | 0 | 0 | 0 | 1980 |
| HORUICHI_WTAP_TARGETS_DN | 224 | 0.5680340 | 2.970475 | 0 | 0 | 0 | 2408 |
| LEE_EARLY_T_LYMPHOCYTE_UP | 78 | 0.5722333 | 2.966888 | 0 | 0 | 0 | 1423 |
| PENG_GLUTAMINE_DEPRIVATION_DN | 243 | 0.5665463 | 2.965871 | 0 | 0 | 0 | 1986 |
| REACTOME_DNA_REPLICATION | 140 | 0.6076682 | 2.961261 | 0 | 0 | 0 | 1989 |
| REACTOME_MITOTIC_M_M_GI_PHASES | 122 | 0.6165375 | 2.961159 | 0 | 0 | 0 | 2050 |
| GRAHAM_NORMAL_QUIESCENT_VS_NORMAL_DIVIDING_ | 72 | 0.6737723 | 2.959949 | 0 | 0 | 0 | 2017 |
| DN | | | | | | | |
| MORI_IMMATURE_B_LYMPHOCYTE_DN | 75 | 0.6602019 | 2.947281 | 0 | 0 | 0 | 2399 |
| FURUKAWA_DUSP6_TARGETS_ [symbol] _DN | 48 | 0.7425728 | 2.9371 | 0 | 0 | 0 | 1333 |
| MORI_LARGE_PRE_BII_LYMPHOCYTE_UP | 71 | 0.6629807 | 2.920429 | 0 | 0 | 0 | 1783 |
| KAUFFMANN_MELANOMA_RELAPSE_UP | 48 | 0.7180015 | 2.918148 | 0 | 0 | 0 | 1754 |
| [symbol] _E2F_TARGETS | 47 | 0.7270096 | 2.919251 | 0 | 0 | 0 | 2244 |
| CHIANG_LIVER_CANCER_SUBLCASS_PROLIFERATION_ | 129 | 0.5999274 | 2.910144 | 0 | 0 | 0 | 2041 |
| UP | | | | | | | |
| MANALO_HYPOXIA_DN | 228 | 0.5593274 | 2.909112 | 0 | 0 | 0 | 2879 |
| LINDGREN_BLADDER_CANCER_CLUSTER_3_UP | 215 | 0.5544321 | 2.904898 | 0 | 0 | 0 | 2885 |
| ZHOU_CELL_CYCLE_GENES_IN_IR_RESPONSE_6HR | 71 | 0.6622609 | 2.893702 | 0 | 0 | 0 | 2244 |
| [symbol] _TRANSFORMED_BY_RHOA_UP | 377 | 0.5261049 | 2.876792 | 0 | 0 | 0 | 2245 |
| ODONNELL_TARGETS_OF_MYC_ANG_TFRC_DN | 37 | 0.7402481 | 2.872902 | 0 | 0 | 0 | 1976 |
| XONG_E2F3_TARGETS | 29 | 0.640658 | 2.849773 | 0 | 0 | 0 | 1980 |
| PUIANA_XPRS5_INT_NETWORK | 128 | 0.5951415 | 2.847488 | 0 | 0 | 0 | 2332 |
| WANG_RESPONSE_TO_GSK3_ [symbol] _ [symbol] _DN | 240 | 0.5399863 | 2.844591 | 0 | 0 | 0 | 1953 |
| REACTOME_CELL_CYCLE | 266 | 0.5292530 | 2.836672 | 0 | 0 | 0 | 2316 |
| TATE_PLASMA_CELL_VS_PLASMABLAST_DN | 222 | 0.5381889 | 2.815545 | 0 | 0 | 0 | 2194 |
| GACIN_FOXP3_TARGETS_CLUSTER_P6 | 56 | 0.664676 | 2.804849 | 0 | 0 | 0 | 1939 |
| [symbol] _PROLIFERATION | 102 | 0.6039325 | 2.802778 | 0 | 0 | 0 | 1980 |
| [symbol] _TARGETS_OF_CCND1_AND_CDKA_DN | 45 | 0.6947311 | 1.793475 | 0 | 0 | 0 | 1899 |
| WHITFIELD_CELL_CYCLE_G2_M | 142 | 0.5631633 | 2.788675 | 0 | 0 | 0 | 2494 |
| YU_MYC_TARGETS_UP | 34 | 0.7430208 | 2.767473 | 0 | 0 | 0 | 1737 |
| PUIANA_BRCA_CENTERED_NETWORK | 94 | 0.6085714 | 2.765684 | 0 | 0 | 0 | 2050 |
| MARKEY_RB1_ACUTE_LOF_UP | 169 | 0.5481748 | 2.748592 | 0 | 0 | 0 | 2836 |
| QU_APOPTOSIS_BY_CDKN1A_VIA_TP53 | 49 | 0.6737988 | 2.7449 | 0 | 0 | 0 | 1813 |
| BENPORAIM_CYCLING_GENES | 430 | 0.4913681 | 2.744477 | 0 | 0 | 0 | 2244 |
| PUIANA_BREAST_CANCER_WITH_BRCA3_MUTATED_UP | 46 | 0.6713083 | 2.73026 | 0 | 0 | 0 | 2308 |
| [symbol] _REGULATED_BY_METHYLATION_DN | 98 | 0.5890535 | 2.728953 | 0 | 0 | 0 | 2244 |
| FRASOR_RESPONSE_TO_ [symbol] _OR_FULVESTRANT_DN | 43 | 0.6845541 | 2.725551 | 0 | 0 | 0 | 1147 |
| [symbol] _BREAST_CANCER_METASTASIS_DN | 84 | 0.5959497 | 2.710735 | 0 | 0 | 0 | 2244 |
| [symbol] _Q4 | 162 | 0.5403296 | 2.708875 | 0 | 0 | 0 | 2332 |
| LINDGREN_BLADDER_CANCER_CLUSTER_1_DN | 237 | 0.52073 | 2.702628 | 0 | 0 | 0 | 2714 |
| TOYOTA_TARGETS_OF_MIR34B_AND_MIR34C | 302 | 0.4961034 | 2.70057 | 0 | 0 | 0 | 2893 |
| KAMMINGA_E2B2_TARGETS | 33 | 0.7312832 | 2.699502 | 0 | 0 | 0 | 2783 |
| KASORELLI_ACUTE_PROMYELOCYTIC_LEUKEMIA_DN | 463 | 0.4728171 | 2.696775 | 0 | 0 | 0 | 2897 |
| HEN_BOUND_BY_E2F | 52 | 0.6574689 | 2.694135 | 0 | 0 | 0 | 2383 |
| STEIN_ [symbol] _TARGETS_RESPONSIVE_TO_ | 31 | 0.7390018 | 2.688479 | 0 | 0 | 0 | 1525 |
| ESTROGEN_DN | | | | | | | |
| E2F_Q5 | 162 | 0.5322467 | 2.679208 | 0 | 0 | 0 | 2332 |
| [symbol] _WILMS_TUMOR_VS_FETAL_KIDNEY_1_DN | 125 | 0.5500801 | 2.673842 | 0 | 0 | 0 | 2399 |
| SONG_TARGETS_OF_IE85_CMV_PROTEIN | 52 | 0.6557211 | 2.667125 | 0 | 0 | 0 | 1055 |
| MOHANKUMAH_TLX1_TARGETS_UP | 274 | 0.5005348 | 2.661168 | 0 | 0 | 0 | 2585 |
| AMUNDSON_GAMMA_RADIATION_RESPONSE | 24 | 0.7198703 | 2.661168 | 0 | 0 | 0 | 1243 |
| WEST_ADRENOCORTICAL_TUMOR_UP | 222 | 0.5045992 | 2.648262 | 0 | 0 | 0 | 2714 |
| REACTOME_MITOTIC_PROMETAPHASE | 66 | 0.6150564 | 2.54542 | 0 | 0 | 0 | 2989 |
| KAUFFMANN_DNA_REPLICATION_GENES | 300 | 0.5681802 | 2.6462 | 0 | 0 | 0 | 2050 |
| BURTON_ADIPOGENESIS_PEAK_AT_16HR | 32 | 0.7150328 | 2.644086 | 0 | 0 | 0 | 2190 |
| WHITFIELD_CELL_CYCLE_LITERATURE | 42 | 0.5811483 | 2.642025 | 0 | 0 | 0 | 1762 |
| PUIANA_BREAST_CANCER_LIT_INT_NETWORK | 73 | 0.5915995 | 2.639938 | 0 | 0 | 0 | 1768 |
| [symbol] _CELL_CYCLE_RB1_TARGETS | 21 | 0.8039092 | 2.537259 | 0 | 0 | 0 | 3248 |
| NAKAYAMA_SOFT_TISSUE_TUMORS_PCA2_UP | 65 | 0.6206203 | 2.637116 | 0 | 0 | 0 | 1731 |
| GOLDRATH_ANTIGEN_RESPONSE | 216 | 0.5083296 | 2.036961 | 0 | 0 | 0 | 2219 |
| SENGUPTA_NASOPHARYNGEAL_CARCINOMA_UP | 191 | 0.5185849 | 2.632345 | 0 | 0 | 0 | 2390 |
| [symbol] _TNC_TARGETS_DN | 109 | 0.5581724 | 2.629792 | 0 | 0 | 0 | 1482 |
| CSR_LATE_UP, V1_UP | 118 | 0.5494502 | 2.629753 | 0 | 0 | 0 | 5042 |
| RODRIGUES_THYROID_CARCINOMA_POORLY_ | 462 | 0.4635281 | 2.627287 | 0 | 0 | 0 | 3092 |
| DIFFERENTIATION_UP | | | | | | | |
| TANG_SENESCENCE_TP53_TARGETS_DN | 46 | 0.5643747 | 2.626405 | 0 | 0 | 0 | 1229 |
| REACTOME_MITOTIC_G1_G1_5_PHASES | 90 | 0.5750307 | 2.625195 | 0 | 0 | 0 | 2640 |
| LE_EGR2_TARGETS_UP | 86 | 0.588038 | 2.625156 | 0 | 0 | 0 | 1810 |
| KHEMNIT2_RESPONSE_TO_PROSTAGLANDIN_E2_UP | 102 | 0.2299114 | 2.623393 | 0 | 0 | 0 | 1737 |
| VECCHI_GASTRIC_CANCER_EARLY_UP | 273 | 0.4884897 | 2.618871 | 0 | 0 | 0 | 2757 |
| E2F4DP1_D1 | 158 | 0.5221322 | 2.61598 | 0 | 0 | 0 | 2215 |
| REACTOME_G2_M_CHECKPOINTS | 35 | 0.5854786 | 2.613967 | 0 | 0 | 0 | 1754 |
| REACTOME_DNA_STRAND_ELONGATION | 26 | 0.7390443 | 2.513621 | 0 | 0 | 0 | 1063 |
| AFFAR_YY1_TARGETS_DN | 141 | 0.5318596 | 2.609855 | 0 | 0 | 0 | 1529 |
| FARMER_BREAST_CANCER_CLUSTER_2 | 28 | 0.7388588 | 2.606811 | 0 | 0 | 0 | 1390 |
| E2F_D2 | 157 | 0.5222832 | 2.603409 | 0 | 0 | 0 | 2219 |
| [symbol] _MALIGNANY_MESOTHELIOMA_UP | 217 | 0.499816 | 2.593973 | 0 | 0 | 0 | 2957 |
| REACTOME_ACTIVATION_OF_THE_PRE_REPLICATE_ | 23 | 0.7647482 | 2.593947 | 0 | 0 | 0 | 926 |
| COMPLEX | | | | | | | |
| PID_AURORA_B_PATHWAY | 29 | 0.7211104 | 2.592803 | 0 | 0 | 0 | 2239 |
| TGASTMAGC_NFE2_D1 | 114 | 0.5554186 | 2.592271 | 0 | 0 | 0 | 2332 |
| E2F4DP2_D1 | 157 | 0.5171896 | 2.591206 | 0 | 0 | 0 | 2219 |
| EDF1_Q6 | 158 | 0.5178882 | 2.591212 | 0 | 0 | 0 | 2219 |
| [symbol] _INVASIVE_BREAST_CANCER_UP | 116 | 0.54061 | 2.589162 | 0 | 0 | 0 | 1737 |
| E2F3DP2_D1 | 152 | 0.5121496 | 2.585663 | 0 | 0 | 0 | 2219 |
| E2F1DPIRB_D1 | 153 | 0.5126388 | 2.57999 | 0 | 0 | 0 | 2332 |
| [symbol] _LIVER_CANCER_SUBLCLASS_ [symbol] _UP | 141 | 0.5222583 | 2.579289 | 0 | 0 | 0 | 3136 |
| REACTOME_CELL_CYCLE_CHECKPOINTS | 78 | 0.565222 | 2.569227 | 0 | 0 | 0 | 2640 |
| REACTOME_5_PHASE | 74 | 0.5798341 | 2.567453 | 0 | 0 | 0 | 2050 |
| REACTOME_G1_S_TRANSITION | 72 | 0.5828485 | 2.55336 | 0 | 0 | 0 | 2640 |
| KEGG_CELL_CYCLE | 93 | 0.5568015 | 2.551866 | 0 | 0 | 0 | 1539 |
| E2F1DP1_D1 | 157 | 0.5171496 | 2.550628 | 0 | 0 | 0 | 2219 |
| REACTOME_SYNTHESIS_OF_DNA | 63 | 0.5880677 | 2.548754 | 0 | 0 | 0 | 2050 |
| SUNG_METASTASIS_STROMA_DN | 33 | 0.6897342 | 2.543812 | 0 | 0 | 0 | 1818 |
| [symbol] | 189 | 0.4952148 | 2.543681 | 0 | 0 | 0 | 2778 |
| REACTOME_M_G1_TRANSITION | 52 | 0.5289346 | 2.541686 | 0 | 0 | 0 | 2640 |
| [symbol] _ERYTHROID_DIFFERENTIATION | 53 | 0.5260986 | 2.541519 | 0 | 0 | 0 | 2174 |
| ERRERT_PROGENITOR | 91 | 0.5486303 | 2.532053 | 0 | 0 | 0 | 2394 |
| NAKAMURA_TUMOR_ZONE_PERIPHERAL_VS_CENTRAL_UP | 165 | 0.5020148 | 2.529642 | 0 | 0 | 0 | 2222 |
| BURTON_ADIPOGENESIS_AT_24HR | 34 | 0.6768556 | 2.529516 | 0 | 0 | 0 | 1402 |
| VEGF_A_UP, V1_DN | 134 | 0.5142433 | 2.523708 | 0 | 0 | 0 | 3110 |
| GAL_LEUKEMIC_STEM_CELL_DN | 105 | 0.5435259 | 2.521694 | 0 | 0 | 0 | 1939 |
| LY_AGING_OLD_DN | 41 | 0.656711 | 2.51529 | 0 | 0 | 0 | 2235 |
| SCIAN_CELL_CYCLE_TARGETS_OF_TP53_AND_TP73_DN | 17 | 0.8156967 | 2.514904 | 0 | 0 | 0 | 1243 |
| REACTOME_ASPARAGINE_N_LINKED_GLYCOSYLATION | 50 | 0.6125944 | 2.496269 | 0 | 0 | 0 | 2416 |
| REICHERT_MITOSIS_ [symbol] _TARGETS | 24 | 0.727967 | 2.496268 | 0 | 0 | 0 | 2173 |
| ELVIDGE_HYPOXIA_DN | 104 | 0.5344598 | 2.492277 | 0 | 0 | 0 | 2483 |
| REACTOME_ACTIVATION_OF_ATR_IN_RESPONSE_TO_ | 29 | 0.5882426 | 2.491589 | 0 | 0 | 0 | 1754 |
| REPLICATION_STRESS | | | | | | | |
| CHANG_CORE_SERUM_RESPONSE_UP | 144 | 0.5119669 | 1.490954 | 0 | 0 | 0 | 3046 |
| REACTOME_PROCESSING_OF_CAPPED_INTRON_ | 94 | 0.5161728 | 2.490088 | 0 | 0 | 0 | 1894 |
| CONTAINING_PRE_MRNA | | | | | | | |
| E2F1_Q3 | 155 | 0.4989949 | 2.489905 | 0 | 0 | 0 | 2219 |
| E2F_01 | 44 | 0.6265794 | 2.489184 | 0 | 0 | 0 | 1566 |
| [symbol] _BREAST_CANCER_PROGNOSIS_UP | 29 | 0.5738477 | 2.486231 | 0 | 0 | 0 | 1875 |
| ZHENG_GLIOBLASTOMA_PLASTICITY_UP | 168 | 0.4924908 | 2.47083 | 0 | 0 | 0 | 1873 |
| PENG_RAPAMYCIN_RESPONSE_DN | 177 | 0.48906024 | 2.464535 | 0 | 0 | 0 | 2786 |
| REACTOME_MITOTIC_G2_G2_M_PHASES | 56 | 0.5810709 | 2.456503 | 0 | 0 | 0 | 2283 |
| [symbol] _G2M_ARREST_BY_2METHOXYESTRADIOL_UP | 73 | 0.5654142 | 2.454489 | 0 | 0 | 0 | 2347 |
| E2F1_UP, V1_UP | 127 | 0.5119882 | 2.452825 | 0 | 0 | 0 | 2553 |
| GARY_CDS_TARGETS_DN | 279 | 0.4585115 | 2.152442 | 0 | 0 | 0 | 3160 |
| BURTON_ADIPOGENESIS_2 | 51 | 0.5927104 | 2.447649 | 0 | 0 | 0 | 1712 |
| [symbol] _PRE_BI_LYMPHOCYTE_UP | 57 | 0.5828884 | 2.446345 | 0 | 0 | 0 | 1783 |
| II_RESPONSE_TO_FSH_DN | 43 | 0.6178514 | 2.425183 | 0 | 0 | 0 | 2381 |
| KAUFFMANN_DNA_REPAIR_GENES | 157 | 0.4887121 | 2.433041 | 0 | 0 | 0 | 3019 |
| CAIRO_HEPATOBLASTOMA_CLASSES_UP | 423 | 0.4375872 | 2.428374 | 0 | 0 | 0 | 2244 |
| E2F_Q4_01 | 147 | 0.4877567 | 2.411003 | 0 | 0 | 0 | 2332 |
| E2F_Q3 | 360 | 0.4781991 | 2.388575 | 0 | 0 | 0 | 2332 |
| GCNP_SHH_UP_LATE, V1_UP | 116 | 0.4964835 | 2.354005 | 0 | 0 | 0 | 2533 |
| E2F_Q6_01 | 152 | 0.4698952 | 2.330773 | 0 | 0 | 0 | 2332 |
| E2F, Q3 | 140 | 0.4718486 | 2.327572 | 0 | 0 | 0 | 2332 |
| E2F_Q3_01 | 148 | 0.4733464 | 2.315264 | 0 | 0 | 0 | 2072 |
| E2F1_Q4_01 | 142 | 0.4668199 | 2.319439 | 0 | 0 | 0 | 2072 |
| E2F1_Q5_01 | 159 | 0.4528135 | 2.271717 | 0 | 0 | 0 | 2215 |
| GARGALOVIC_RESPONSE_TO_OXIDIZED_ | 34 | 0.6431882 | 2.381599 | 0 | 6.94E−06 | 0.001 | 1965 |
| PHOSPHOLIPIDS_TURQUOISE_DN | | | | | | | |
| GRAHAM_CML_QUIESCENT_VS_NORMAL_QUIESCENT_ | 53 | 0.5729029 | 2.385525 | 0 | 6.99E−06 | 0.001 | 1737 |
| UP | | | | | | | |
| SU_TESTIS | 43 | 0.5052453 | 2.38601 | 0 | 7.03E−06 | 0.001 | 2583 |
| PATIL_LIVER_CANCER | 491 | 0.4270232 | 2.389805 | 0 | 7.08E−06 | 0.001 | 2894 |
| VERNELL_RETINOBLASTOME_PATHWAY_UP | 62 | 0.5558563 | 2.390926 | 0 | 7.13E−06 | 0.001 | 2017 |
| WONG_EMBRYONIC_STEM_CELL_CORE | 237 | 0.4531235 | 2.394108 | 0 | 7.18E−06 | 0.001 | 1848 |
| [symbol] _DNA_REPLICATION | 27 | 0.6996888 | 2.396272 | 0 | 7.23E−06 | 0.001 | 1754 |
| [symbol] _EMU_MYC_LYMPHOMA_BY_ONSET_TIME_UP | 65 | 0.5518671 | 2.3972 | 0 | 7.28E−06 | 0.001 | 2879 |
| KEGG_PROGESTERONE_MEDIATED_OOCYTE_ | 49 | 0.5913903 | 2.401491 | 0 | 7.33E−06 | 0.001 | 1553 |
| MATURATION | | | | | | | |
| OSMAN_BLADDER_CANCER_UP | 255 | 0.4531255 | 2.404237 | 0 | 7.38E−06 | 0.001 | 2631 |
| [symbol] _PLK_PATHWAY | 36 | 0.6268197 | 2.407149 | 0 | 7.44E−06 | 0.001 | 1243 |
| WILCOX_RESPONSE_TO_PROGESTERONE_UP | 96 | 0.5241065 | 2.412156 | 0 | 7.49E−06 | 0.001 | 1762 |
| KEGG_OOCYTE_MEIOSIS | 63 | 0.5720019 | 2.414789 | 0 | 7.54E−06 | 0.001 | 2610 |
| [symbol] _WILMS_TUMOR_ANAPLASTIC_UP | 18 | 0.7413286 | 2.351513 | 0 | 1.84E−05 | 0.002 | 1223 |
| [symbol] _CELL_CYCLE_G2 | 119 | 0.4957912 | 2.557137 | 0 | 1.35E−05 | 0.002 | 1456 |
| MONNIER_POSTRADIATION_TUMOR_ESCAPE_UP | 257 | 0.4436246 | 2.3585 | 0 | 1.36E−05 | 0.002 | 3483 |
| [symbol] _BREAST_CANCER_ [symbol] | 16 | 0.7686798 | 2.3583 | 0 | 1.36E−05 | 0.002 | 2243 |
| REACTOME_MRNA_PROCESSING | 107 | 0.5003405 | 2.364106 | 0 | 1.38E−05 | 0.002 | 1981 |
| REACTOME_TRANSPORT_OF_MATURE_TRANSCRIPT_ | 39 | 0.615582 | 2.364106 | 0 | 1.38E−05 | 0.002 | 1848 |
| TO_CYTOPLASM | | | | | | | |
| [symbol] _CTNNB1_TARGETS_DN | 368 | 0.4308235 | 2.378087 | 0 | 1.39E−05 | 0.002 | 2573 |
| [symbol] _LIVER_CANCER_SUBCLASS_G23_UP | 40 | 0.6095077 | 2.347648 | 0 | 2.00E−05 | 0.003 | 1038 |
| RODRIGUES_THYROID_CARCINOMA_ANAPLASTIC_UP | 492 | 0.4135969 | 2.309747 | 0 | 2.50E−05 | 0.004 | 2647 |
| RHODES_UNDIFFERENTIATED_CANCER | 50 | 0.5741092 | 2.312351 | 0 | 2.52E−05 | 0.004 | 1771 |
| STEIN_ESR1_TARGETS | 57 | 0.5478188 | 2.320627 | 0 | 2.53E−05 | 0.004 | 1525 |
| PID_ATR_PATHWAY | 34 | 0.6201547 | 2.528239 | 0 | 2.55E−05 | 0.004 | 1768 |
| REACTOME_ASSEMBLY_OF_THE_PRE_REPLICATIVE_ | 42 | 0.5948156 | 2.328291 | 0 | 2.56E−05 | 0.004 | 2640 |
| COMPLEX | | | | | | | |
| [symbol] _MCM_PATHWAY | 15 | 0.7687059 | 1.331338 | 0 | 2.58E−05 | 0.004 | 256 |
| REACTOME_TRANSPORT_TO_THE_GOLGI_AND_ | 25 | 0.6869984 | 2.531537 | 0 | 2.59E−05 | 0.004 | 1495 |
| SUBSEQUENT_MODIFICATION | | | | | | | |
| [symbol] _LIVER_CANCER_SUBCLASS_G123_UP | 33 | 0.6305398 | 2.33323 | 0 | 2.61E−05 | 0.004 | 3070 |
| PID_ [symbol] _PATHWAY | 50 | 0.5685199 | 2.53457 | 0 | 2.63E−05 | 0.004 | 2769 |
| ZHANG_BREAST_CANCER_PROGENITORS_UP | 279 | 0.434684 | 2.34155 | 0 | 2.64E−05 | 0.004 | 2426 |
| WHITFIELD_CELL_CYCLE_G1_ [symbol] | 96 | 0.5123215 | 2.341555 | 0 | 2.66E−05 | 0.004 | 2877 |
| REACTOME_ANTIVIRAL_MECHANISM_BY_IFN_ | 51 | 0.5613973 | 2.297269 | 0 | 3.07E−05 | 0.005 | 3136 |
| STIMUATED_GENES | | | | | | | |
| REACTOME_REGULATION_OF_MITOTIC_CELL_CYCLE | 48 | 0.5779655 | 2.298501 | 0 | 3.09E−05 | 0.005 | 2627 |
| PAL_PRMTS_TARGETS_UP | 124 | 0.480239 | 2.299389 | 0 | 3.10E−05 | 0.005 | 2571 |
| [symbol] _UV_RESPONSE_KERATINOCYTE_ [symbol] | 328 | 0.4257683 | 2.291295 | 0 | 3.61E−05 | 0.005 | 3038 |
| GEORGES_CELL_CYCLE_MIR192_TARGETS | 49 | 0.5611235 | 2.29236 | 0 | 3.63E−05 | 0.006 | 2001 |
| VANIVEER_BREAST_CANCER_ESR1_DN | 151 | 0.4659884 | 2.294941 | 0 | 3.65E−05 | 0.006 | 1732 |
| REACTOME_MNC_CLASS_II_ANTIGEN_PRESENTATION | 61 | 0.5386897 | 2.271817 | 0 | 4.08E−05 | 0.007 | 2329 |
| REACTOME_G1_S_SPECIFIC_TRANSCRIPTION | 15 | 0.758102 | 2.273017 | 0 | 4.20E−05 | 0.007 | 857 |
| [symbol] _B_CLL_WITH_VH3_21_ [symbol] | 35 | 0.6047155 | 2.275127 | 0 | 4.13E−05 | 0.007 | 2362 |
| PID_FOXM3_PATHWAY | 30 | 0.6251206 | 2.275983 | 0 | 4.15E−05 | 0.007 | 1525 |
| REACTOME_KINESINS | 18 | 0.7059842 | 2.280051 | 0 | 4.17E−05 | 0.007 | 736 |
| REACTOME_MRNA_SPLICING | 71 | 0.5256779 | 2.289946 | 0 | 4.20E−05 | 0.007 | 1894 |
| DACOSTA_UV_RESPONSE_VIA_ERCC3_COMMON_DN | 332 | 0.413126 | 2.258007 | 0 | 4.63E−05 | 0.008 | 3146 |
| IGCACTI_ [symbol] | 266 | 0.4066616 | 2.1657 | 0 | 5.11E−05 | 0.001 | 2292 |
| PYEON_CANCER_HEAD_AND_NECK_VS_CERVICAL_UP | 125 | 0.470616 | 2.261943 | 0 | 5.14E−05 | 0.009 | 2931 |
| [symbol] _NEOPLASTIC_TRANSFORMATION_KRAS_UP | 81 | 0.5071876 | 2.262118 | 0 | 5.17E−05 | 0.009 | 2737 |
| [symbol] _TARGETS_DN | 217 | 0.4301757 | 2.236008 | 0 | 5.82E−05 | 0.001 | 2126 |
| REACTOME_RECRUITMENT_OF_MITOTIC_ | 45 | 0.559968 | 2.240593 | 0 | 5.85E−05 | 0.011 | 2281 |
| CENTROSOME_PROTEINS_AND_COMPLEXES | | | | | | | |
| [symbol] _INTERACT_WITH_ [symbol] | 38 | 0.5720327 | 2.241129 | 0 | 5.88E−05 | 0.011 | 1885 |
| REACTOME_ [symbol] _OF_NUP_FROM_MITOTIC_ | 41 | 0.5645759 | 2.241887 | 0 | 5.91E−05 | 0.011 | 2281 |
| CENTROSOMES | | | | | | | |
| [symbol] _APOPTOSIS_VIA_CD40_UP | 129 | 0.4596237 | 2.24277 | 0 | 5.94E−05 | 0.011 | 1817 |
| REACTOME_FORMATION_OF_ [symbol] _FOLDING_ | 17 | 0.7198963 | 2.247552 | 0 | 5.97E−05 | 0.011 | 2626 |
| INTERMEDIATES_BY_ [symbol] | | | | | | | |
| [symbol] _BREAST_CANCER_LUMINAL_VS_ | 304 | 0.4141847 | 2.24982 | 0 | 6.00E−05 | 0.011 | 2233 |
| MESENCHYMAL_DN | | | | | | | |
| WELSCH_BRCA1_TARGETS_DN | 109 | 0.4808689 | 2.249974 | 0 | 6.03E−05 | 0.011 | 1984 |
| [symbol] _AML_BY_ [symbol] _LOCALIZATION_DN | 117 | 0.4726266 | 2.26075 | 0 | 6.06E−05 | 0.011 | 1762 |
| WHITFIELD_CELL_CYCLE_5 | 101 | 0.4808036 | 2.252008 | 0 | 5.09E−05 | 0.011 | 2050 |
| [symbol] _GLUCOCORTICOID_THERAPY_DN | 234 | 0.4290472 | 2.252641 | 0 | 6.13E−05 | 0.011 | 2786 |
| [symbol] _RB1_TARGETS_SENESCENT | 338 | 0.4124367 | 2.253956 | 0 | 6.16E−05 | 0.011 | 1980 |
| [symbol] _E2F3_ONCOGENIC_SIGNATURE | 161 | 0.4473504 | 2.256184 | 0 | 6.19E−05 | 0.011 | 2498 |
| REACTOME_E2F_MEDIATED_REGULATION_OF_DNA_ | 25 | 0.5585140 | 2.256432 | 0 | 6.23E−05 | 0.011 | 1245 |
| REPLICATION | | | | | | | |
| [symbol] _RESPONSE_TO_GONADOTRPHINS_UP | 58 | 0.5308755 | 2.225892 | 0 | 5.31E−05 | 0.012 | 1766 |
| [symbol] _HDAC1_TARGETS_UP | 277 | 0.4140967 | 2.222115 | 0 | 2.26E−05 | 0.014 | 3208 |
| [symbol] _PSEUDOPODIA_HAPTOTAXIS_DN | 438 | 0.395995 | 2.2225 | 0 | 2.30E−05 | 0.014 | 3049 |
| ONDER_CDH1_TARGETS_1_DN | 89 | 0.4845379 | 2.224719 | 0 | 7.22E−05 | 0.014 | 1741 |
| SASAKI_ADULT_T_CELL_LEUKEMIA | 113 | 0.4693115 | 2.13253 | 0 | 7.73E−05 | 0.015 | 3371 |
| SCHMIDT_FOR_TARGETS_IN_LIMB_BUD_UP | 20 | 0.6919208 | 2.231637 | 0 | 8.16E−05 | 0.016 | 1870 |
| PID_PDGFRB_PATHWAY | 88 | 0.4862162 | 2.232389 | 0 | 8.20E−05 | 0.016 | 2261 |
| REACTOM_GLUCONE_TRANSPORT | 26 | 0.6342709 | 2.21083 | 0 | 8.64E−05 | 0.017 | 1848 |
| TBK1DF_DN | 185 | 0.4340437 | 2.200987 | 0 | 1.01E−04 | 0.001 | 2610 |
| [symbol] _MDM4_TARGETS_FETAL_LIVER_DN | 321 | 0.4046304 | 2.198309 | 0 | 1.16E−04 | 0.023 | 3220 |
| [symbol] _OVARIAN_CANCER_INVAVSIVE_VS_LMP_UP | 87 | 0.4908394 | 2.190942 | 0 | 1.20E−04 | 0.024 | 2167 |
| ELVIDGE_HIF1A_TARGETS_UP | 47 | 0.5452291 | 2.195089 | 0 | 1.20E−04 | 0.024 | 3177 |
| [symbol] _UP | 82 | 0.4941093 | 2.215586 | 0 | 1.23E−04 | 0.001 | 1899 |
| MEINHOLD_OVARIAN_CANCER_LOW_GRADE_DN | 15 | 0.7217879 | 2.197403 | 0 | 1.21E−04 | 0.024 | 1218 |
| KEGG_CITRATE_CYCLE_TCA_CYCLE | 23 | 0.649862 | 2.188362 | 0 | 1.23E−04 | 0.025 | 1825 |
| REACTOME_ [symbol] _INFECTION | 117 | 0.4638732 | 2.188439 | 0 | 1.24E−04 | 0.025 | 2512 |
| REACTOME_TRANSPORT_OF_MATURE_MRNA_DERIVED_ | 25 | 0.6257544 | 2.187429 | 0 | 1.28E−04 | 0.026 | 1848 |
| FROM_AN_INTRONLESS_TRANSCRIPT | | | | | | | |
| MARTINEZ_RESPONSE_TO_TRABECTEDIN_DN | 173 | 0.4324481 | 2.183882 | 0 | 1.30E−04 | 0.027 | 3565 |
| MID_TCR_PATHWAY | 30 | 0.500023 | 2.184291 | 0 | 1.31E−04 | 0.027 | 2335 |
| PIC_AURORA_A_PATHWAY | 22 | 0.6657441 | 2.185917 | 0 | 1.31E−04 | 0.027 | 2674 |
| REACTOME_APC_C_CDH1_MEDIATED_DEGRADATION_ | 38 | 0.5599772 | 2.186917 | 0 | 1.32E−04 | 0.027 | 2627 |
| OF_CDC20_AND_OTHER_APC_C_CDH1_TARGETED_ | | | | | | | |
| PROTEINS_IN_LATE_MITOSIS_EARLY_G1 | | | | | | | |
| [symbol] _B_LYMPHOCYTE_NETWORK | 91 | 0.4691338 | 2.175064 | 0 | 1.33E−04 | 0.028 | 1434 |
| SENESE_HDAC2_TARGETS_UP | 69 | 0.4932556 | 2.178892 | 0 | 1.33E−04 | 0.028 | 2516 |
| GROSS_HYPOXIA_VIA_ELK3_UP | 136 | 0.44319 | 2.178925 | 0 | 1.34E−04 | 0.028 | 2219 |
| DOUGLAS_ [symbol] _TARGETS_UP | 360 | 0.396865 | 2.179383 | 0 | 1.34E−04 | 0.028 | 1909 |
| REACTOME_NEP_NS2_INTERACTS_WITH_THE_ | 21 | 0.6471572 | 2.155343 | 0 | 1.39E−04 | 0.03 | 1848 |
| CELLULAR_EXPORT_MACHINERY | | | | | | | |
| KEGG_PATHOGENIC_ESCHERICHIA_COLI_INFECTION | 35 | 0.5693248 | 2.166747 | 0 | 1.40E−04 | 0.03 | 1552 |
| JIANG_VHL_TARGETS | 80 | 0.4892545 | 2.171472 | 0 | 1.40E−04 | 0.03 | 2499 |
| PIC_E2F_PATHWAY | 58 | 0.52042 | 2.171815 | 0 | 1.41E−04 | 0.03 | 875 |
| FERRANDO_T_ALL_WITH_MLL_ENL_FUSION_DN | 55 | 0.5186248 | 2.174135 | 0 | 1.41E−04 | 0.03 | 1959 |
| KEGG_FC_GAMMA_ [symbol] _PHAGOCYTOSIS | 56 | 0.5262478 | 2.153769 | 0 | 1.43E−04 | 0.031 | 2529 |
| [symbol] _E5_2 | 23 | 0.5457991 | 2.160506 | 0 | 1.47E−04 | 0.032 | 1867 |
| [symbol] _SOX4_TARGETS_DN | 38 | 0.5652034 | 2.15772 | 0 | 1.51E−04 | 0.033 | 2102 |
| REACTOME_DOUBLE_STRAND_BREAK_REPAIR | 18 | 0.68211 | 2.155988 | 0 | 1.64E−04 | 0.036 | 1768 |
| IVANOVA_HEMATOPOIESIS_LATE_PROGENITOR | 264 | 0.4037208 | 2.155303 | 0 | 1.58E−04 | 0.037 | 3071 |
| GARCIA_TARGETS_OF_ [symbol] _AND_DAX1_DN | 163 | 0.4627588 | 2.152591 | 0 | 1.80E−04 | 0.04 | 1939 |
| [symbol] _RHO_PATHWAY | 25 | 0.6245347 | 2.153595 | 0 | 1.81E−04 | 0.04 | 1939 |
| REACTOME_FACTORS_INVOLVED_IN_ | 55 | 0.4943024 | 2.34644 | 0 | 1.92E−04 | 0.043 | 2615 |
| MEGAKARYOCYTE_DEVELOPMENT_AND_PLATELET_ | | | | | | | |
| PRODUCTION | | | | | | | |
| WHITFIELD_CELL_CYCLE_M_G2 | 89 | 0.474211 | 2.151257 | 0 | 1.92E−04 | 0.043 | 3022 |
| REACTOME_METABOLISM_OF_NON_CODING_RNA | 32 | 0.5724509 | 2.141542 | 0 | 2.03E−04 | 0.046 | 1848 |
| [symbol] _VS_FOLLICULAR_LYMPHOMA_UP | 35 | 0.5724509 | 2.142214 | 0 | 2.03E−04 | 0.046 | 2125 |
| REACTOME_APC_C_CDC20_MEDIATED_DEGRADATION_ | 39 | 0.5517606 | 2.14224 | 0 | 2.04E−04 | 0.046 | 2627 |
| OF_MITOTIC_PROTEINS | | | | | | | |
| REACTOME_CHOLESTEROL_BIOSYNTHESIS | 18 | 0.6780912 | 2.138251 | 0 | 2.09E−04 | 0.048 | 2216 |
| [symbol] _ESTRADIOL_RESPONSE_ [symbol] _UP | 163 | 0.4279309 | 2.13847 | 0 | 2.10E−04 | 0.048 | 1427 |
| REACTOME_HOST_INTERACTIONS_HIV_FACTORS | 73 | 0.4871194 | 2.139001 | 0 | 2.10E−04 | 0.048 | 2443 |
| [symbol] _APOPTOSIS_BY_REOVIRUS_INFECTION_DN | 211 | 0.4087728 | 2.136984 | 0 | 2.12E−04 | 0.049 | 2093 |
| [symbol] _RESPONSE_TO_IR_ [symbol] _DN | 72 | 0.4874165 | 2.135013 | 0 | 2.16E−04 | 0.049 | 2310 |
| WAKASUGI_HAVE_ZNF143_BINDING_SITES | 40 | 0.5463916 | 2.134691 | 0 | 2.18E−04 | 0.051 | 1085 |
| [symbol] _NEUROBLASTOMA_COPY_NUMBER_DN | 446 | 0.3819454 | 2.134862 | 0 | 2.19E−04 | 0.051 | 3027 |
| [symbol] _RESPONSE_TO_THC_DN | 24 | 0.6253232 | 2.134149 | 0 | 2.21E−04 | 0.052 | 1473 |
| [symbol] | 134 | 0.4368284 | 2.127377 | 0 | 2.26E−04 | 0.005 | 2122 |
| MARIADASON_REGULATED_BY_HISTONE_ | 27 | 0.5871342 | 2.132109 | 0 | 2.32E−04 | 0.053 | 1437 |
| ACETYLATION_DN | | | | | | | |
| JOHNSTONE_ [symbol] _TARGETS_2_DN | 206 | 0.4127925 | 2.132638 | 0 | 2.33E−04 | 0.053 | 2470 |
| SP1_Q4_01 | 135 | 0.4345556 | 2.133049 | 0 | 2.37E−04 | 0.005 | 2258 |
| CHIANG_LIVER_CANCER_SUBCLASS_UNANNOTATED_ | 119 | 0.4435938 | 2.129865 | 0 | 2.42E−04 | 0.056 | 3818 |
| DN | | | | | | | |
| REACTOME_ADAPTIVE_IMMUNE_SYSTEM | 279 | 0.3978644 | 2.130015 | 0 | 2.43E−04 | 0.056 | 2360 |
| [symbol] _INVASION_INHIBITED_BY_ASCITES_UP | 61 | 0.5104208 | 2.126163 | 0 | 2.66E−04 | 0.062 | 2244 |
| [symbol] _HYPOXIA_NOT_VIA_ [symbol] | 459 | 0.3777474 | 2.124628 | 0 | 2.68E−04 | 0.063 | 2684 |
| SHEPARD_BMYB_TARGETS | 51 | 0.5122973 | 2.124899 | 0 | 2.69E−04 | 0.063 | 2684 |
| PODAR_RESPONSE_TO_ADAPHOSTIN_DN | 16 | 0.6921382 | 2.116942 | 0 | 3.07E−04 | 0.071 | 864 |
| REACTOME_ACTIVATION_OF_CHAPERONE_GENES_ | 29 | 0.5858846 | 2.116321 | 0 | 3.13E−04 | 0.073 | 2137 |
| BY_XBP15 | | | | | | | |
| REACTOME_G0_AND_EARLY_G1 | 18 | 0.5589424 | 2.109192 | 0 | 3.49E−04 | 0.082 | 2610 |
| [symbol] _TARGETS_DN | 56 | 0.4985609 | 2.107092 | 0 | 3.59E−04 | 0.085 | 3054 |
| REACTOME_ [symbol] _REMOVAL_FROM_CHROMATIN | 42 | 0.5346347 | 2.104473 | 0 | 3.70E−04 | 0.087 | 2640 |
| LY_AGING_PREMATURE_DN | 23 | 0.62672 | 2.102505 | 0 | 3.77E−04 | 0.089 | 2233 |
| PIC_FCER1_PATHWAY | 33 | 0.5656276 | 2.101537 | 0 | 3.83E−04 | 0.091 | 2529 |
| [symbol] _EARLY_ [symbol] _UP | 97 | 0.4600552 | 2.223146 | 0 | 2.85E−04 | 0.004 | 3200 |
| REACTOME_DNA_REPAIR | 68 | 0.4835479 | 2.100369 | 0 | 3.85E−04 | 0.092 | 1886 |
| PIC_FANCONE_PATHWAY | 35 | 0.5660565 | 2.096959 | 0 | 3.92E−04 | 0.094 | 2089 |
| [symbol] | 240 | 0.399723 | 2.092987 | 0 | 4.10E−04 | 0.098 | 2778 |
| [symbol] | 120 | 0.4491469 | 2.132656 | 0 | 4.20E−04 | 0.005 | 1939 |
| [symbol] _TARGETS | 129 | 0.4327073 | 2.091803 | 0 | 4.20E−04 | 0.101 | 3168 |
| WINTER_HYPOXIA_UP | 56 | 0.5079345 | 2.091212 | 0 | 4.23E−04 | 0.102 | 2586 |
| [symbol] | 419 | 0.3720492 | 2.070286 | 0 | 4.30E−04 | 0.01 | 2521 |
| OXFORD_RALA_OR_RALB_TARGETS_UP | 39 | 0.5439909 | 2.088946 | 0 | 4.37E−04 | 0.106 | 1053 |
| KEGG_AMINO_SUGAR_AND_NUCLEOTIDE_SUGAR_ | 26 | 0.5975822 | 2.087967 | 0 | 4.43E−04 | 0.108 | 2238 |
| METABOLISM | | | | | | | |
| SASSON_RESPONSE_TO_FORSKOLIN_UP | 58 | 0.493644 | 2.08704 | 0 | 4.49E−04 | 0.11 | 2533 |
| KIN_APC_TARGETS | 43 | 0.5227274 | 2.085796 | 0 | 4.63E−04 | 0.144 | 1651 |
| [symbol] _ALVEOLAR_RHABDOMYOSARCOMA_DN | 276 | 0.8926061 | 2.083815 | 0 | 4.77E−04 | 0.118 | 2738 |
| [symbol] _ACINAR_DEVELOPMENT_LATE_DN | 19 | 0.6474959 | 2.072123 | 0 | 4.90E−04 | 0.121 | 851 |
| PBC2_ [symbol] _DN | 134 | 0.4268149 | 2.076228 | 0 | 5.13E−04 | 0.007 | 2312 |
| CABP_B | 143 | 0.4189898 | 2.093228 | 0 | 5.32E−04 | 0.013 | 2829 |
| THEILGAARD_NEUTROPHIL_AT_SKIN_WOUND_DN | 125 | 0.4324535 | 2.065624 | 0 | 5.26E−04 | 0.151 | 2186 |
| REACTOME_LICAM_INTERACTIONS | 53 | 0.4901342 | 2.053811 | 0 | 5.35E−04 | 0.153 | 2277 |
| RACTOME_IMMUNE_SYSTEM | 447 | 0.3563814 | 2.06316 | 0 | 6.48E−04 | 0.156 | 2335 |
| REACTOME_EXTENSION_OF_TELOMERES | 21 | 0.5127662 | 2.061614 | 0 | 6.65E−04 | 0.161 | 2995 |
| REACTOME_PREFOLDIN_MEDIATED_TRANSFER_OF_ | 21 | 0.5276293 | 2.058993 | 0 | 7.00E−04 | 0.167 | 2626 |
| SUBSTRATE_TO_CCT_TRIC | | | | | | | |
| [symbol] _PROLIFERATION_CLUSTER_DN | 53 | 0.4996137 | 2.05855 | 0 | 7.08E−04 | 0.17 | 2610 |
| CHICAS_RB1_TARGETS_GROWING | 158 | 0.4130699 | 2.057432 | 0 | 7.17E−04 | 0.173 | 1248 |
| SHEPARD_CRUSH_AND_BURN_MUTANT_DN | 115 | 0.4365805 | 2.055288 | 0 | 7.41E−04 | 0.18 | 1506 |
| PIC_DCD42_REG_PATHWAY | 23 | 0.5967745 | 2.051288 | 0 | 7.79E−04 | 0.19 | 2091 |
| SAKAL_CHRONIC_HEPATITIS_VS_LIVER_CANCER_UP | 56 | 0.4953023 | 2.051345 | 0 | 7.82E−04 | 0.19 | 3121 |
| GRUETZMANN_PANCREATIC_CANCER_UP | 228 | 0.3930303 | 2.046502 | 0 | 8.39E−04 | 0.204 | 2331 |
| REACTOME_HIV_LIFE_CYCLE | 24 | 0.4655075 | 2.045835 | 0 | 8.43E−04 | 0.204 | 2532 |
| [symbol] _TARGETS_DN | 422 | 0.367635 | 2.044175 | 0 | 8.81E−04 | 0.213 | 2579 |
| PETROVA_ENDOTHELIUM_LYMPHATIC_VS_BLOOD_UP | 79 | 0.4603269 | 2.043856 | 0 | 8.81E−04 | 0.214 | 1582 |
| ELK1_02 | 125 | 0.4157337 | 2.006724 | 0 | 9.40E−04 | 0.024 | 3202 |
| WANG_CISPLATIN_RESPONSE_AND_XPC_UP | 112 | 0.4272534 | 2.032858 | 0 | 9.47E−04 | 0.231 | 2264 |
| KEGG_ [symbol] _MEDIATED_PROTEOLYSIS | 89 | 0.4459266 | 2.036673 | 0 | 9.59E−04 | 0.234 | 2913 |
| RECTOME_CITRIC_ACID_CYCLE_TCA_CYCLE | 15 | 0.6613815 | 2.035854 | 0 | 9.66E−04 | 0.237 | 1824 |
| CINTILE_RESPONSE_CLUSTER_D3 | 51 | 0.4862784 | 2.03346 | 0 | 9.78E−04 | 0.239 | 3077 |
| WSIT_ADRENOCORTICAL_TUMOR_MARKERS_UP | 17 | 0.6444951 | 2.034373 | 0 | 9.95E−04 | 0.244 | 1232 |

[symbol]

indicates data missing or illegible when filed

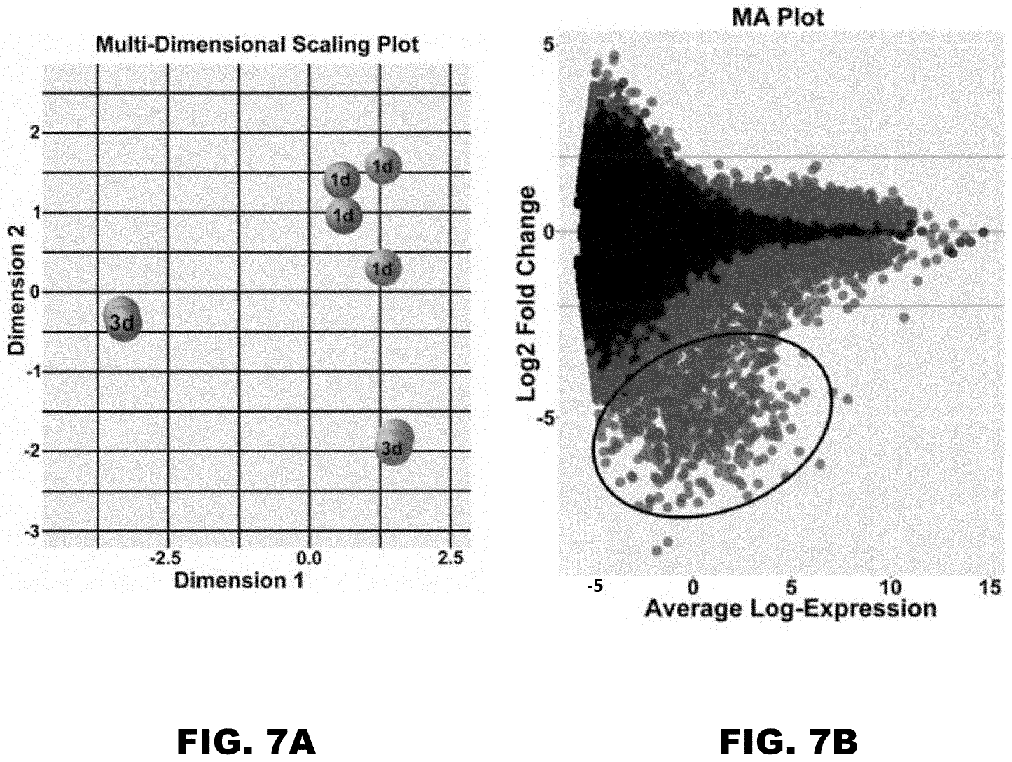

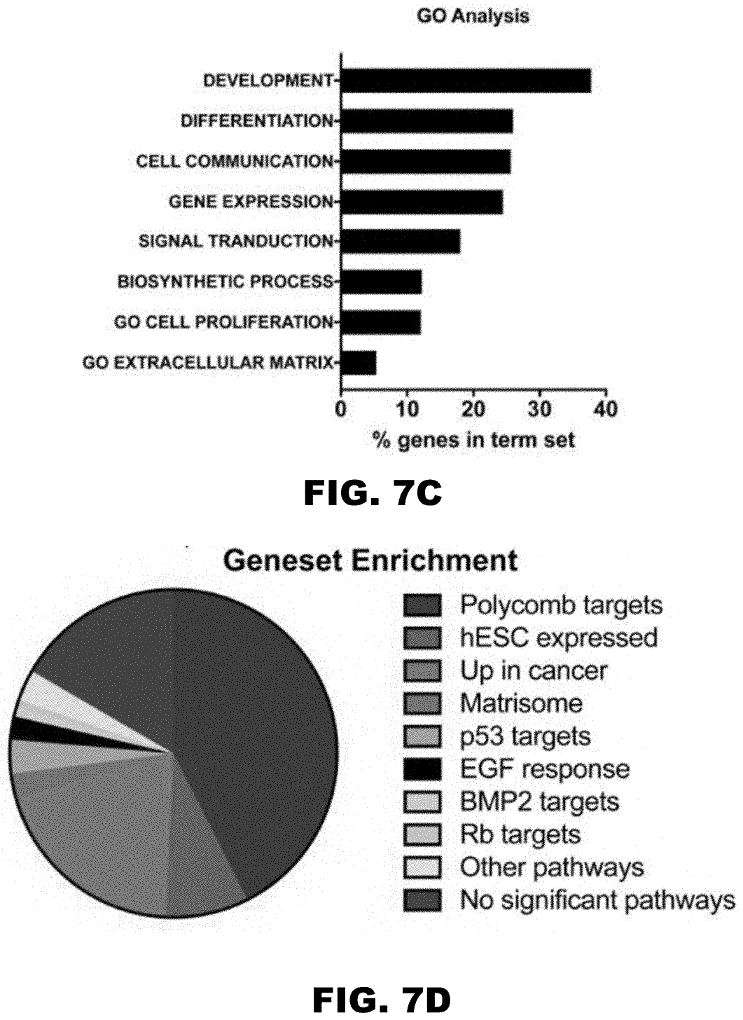

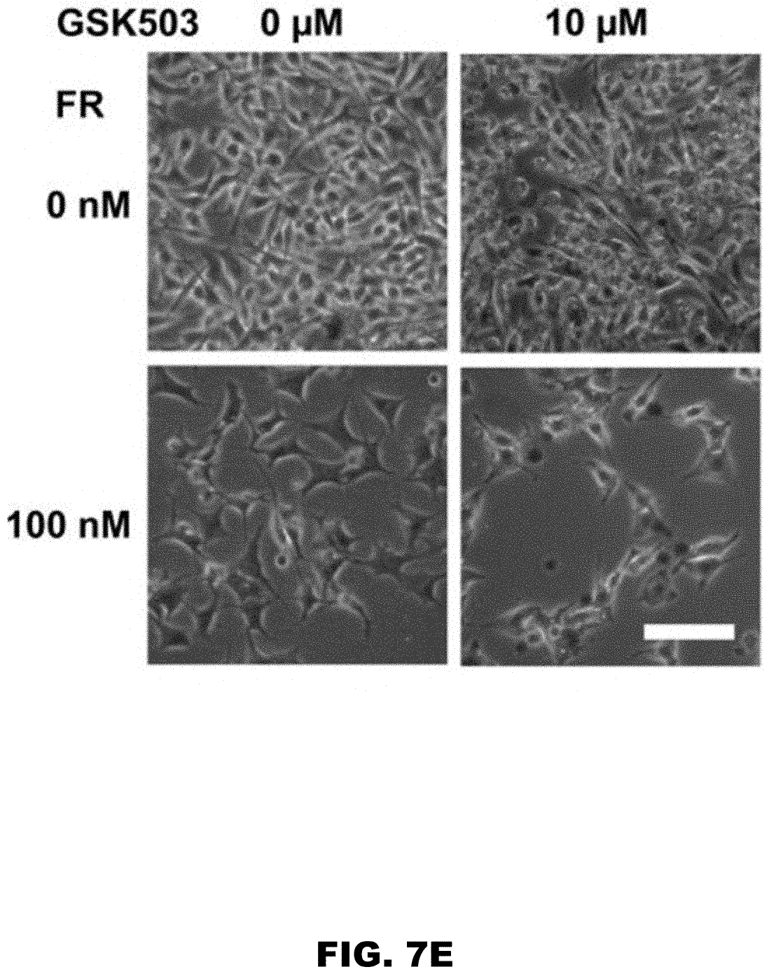

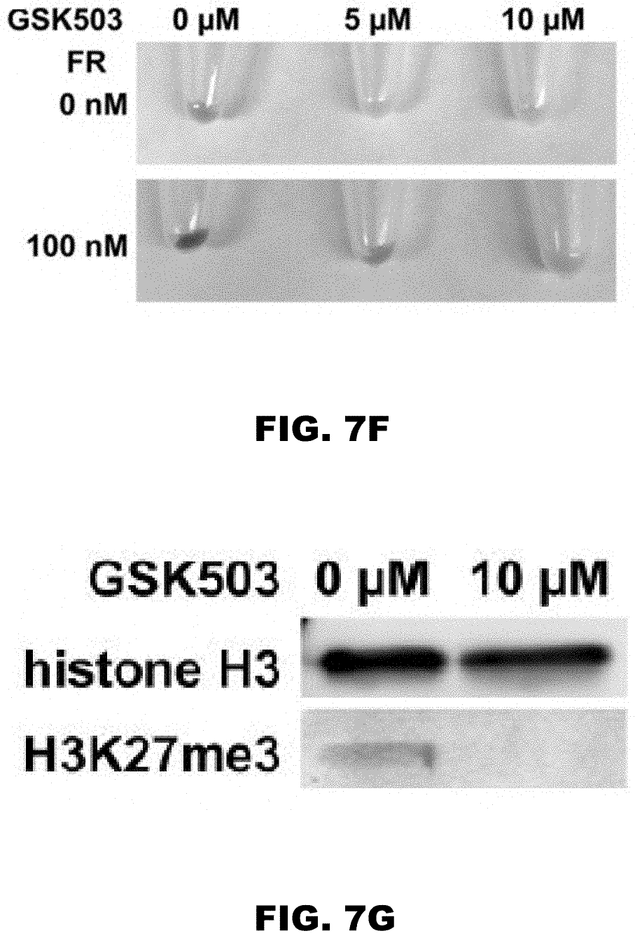

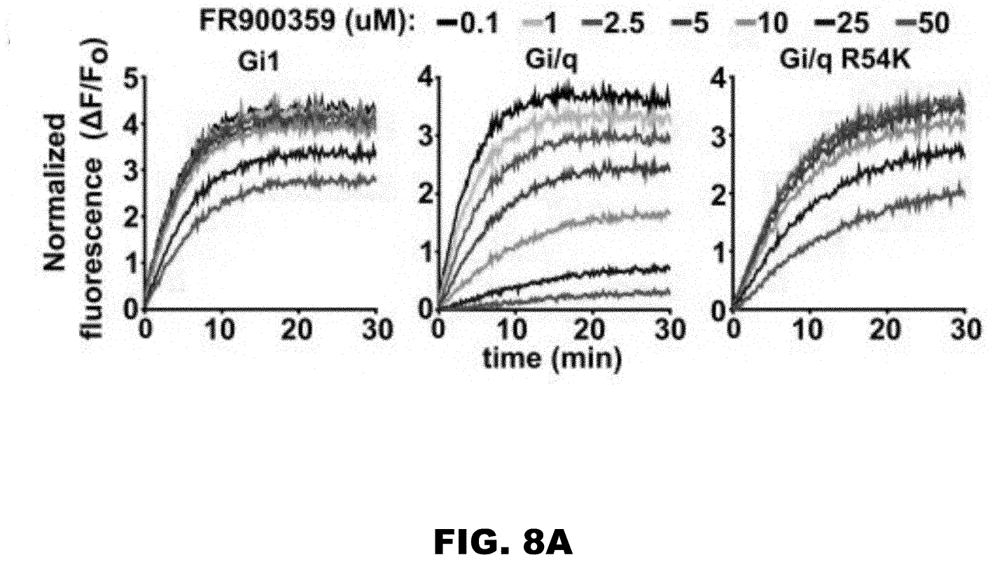

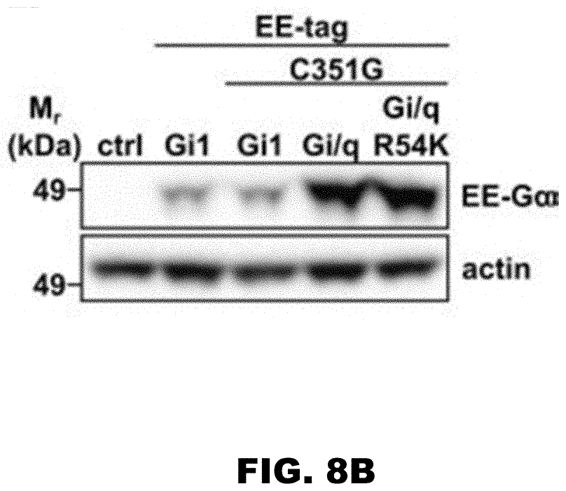

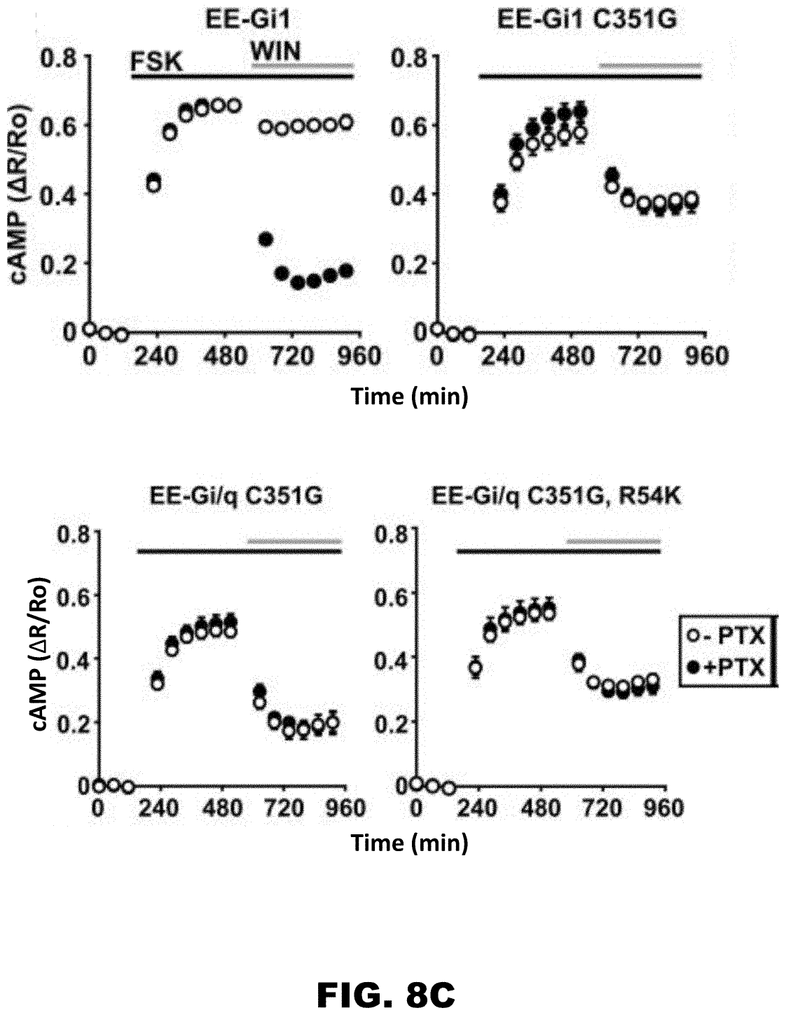

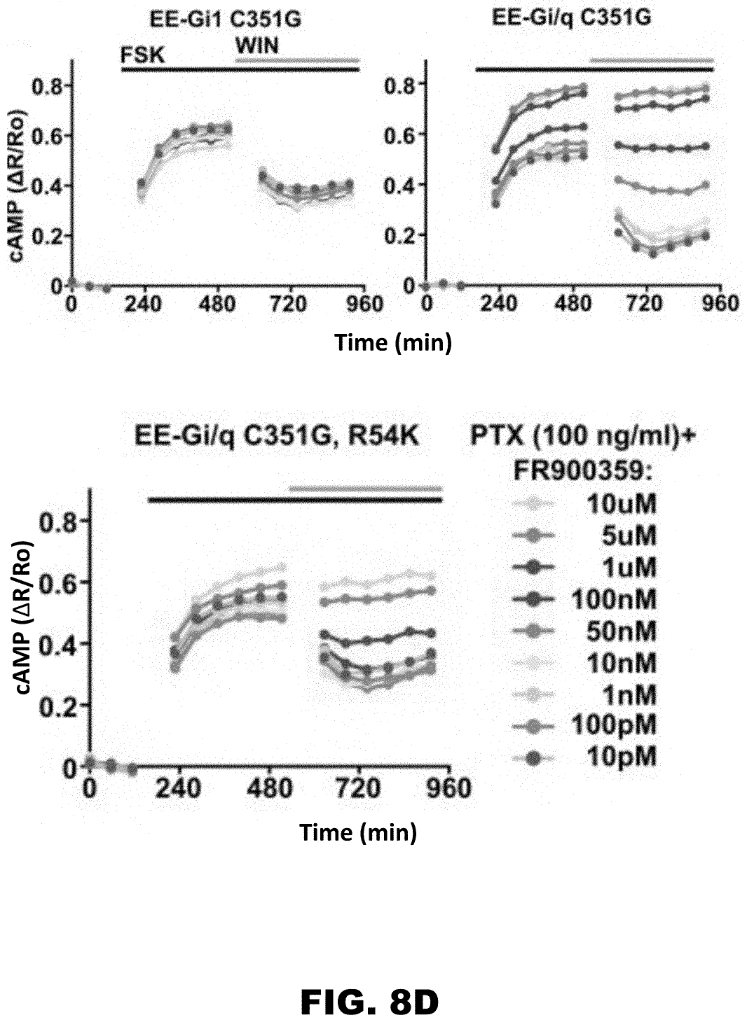

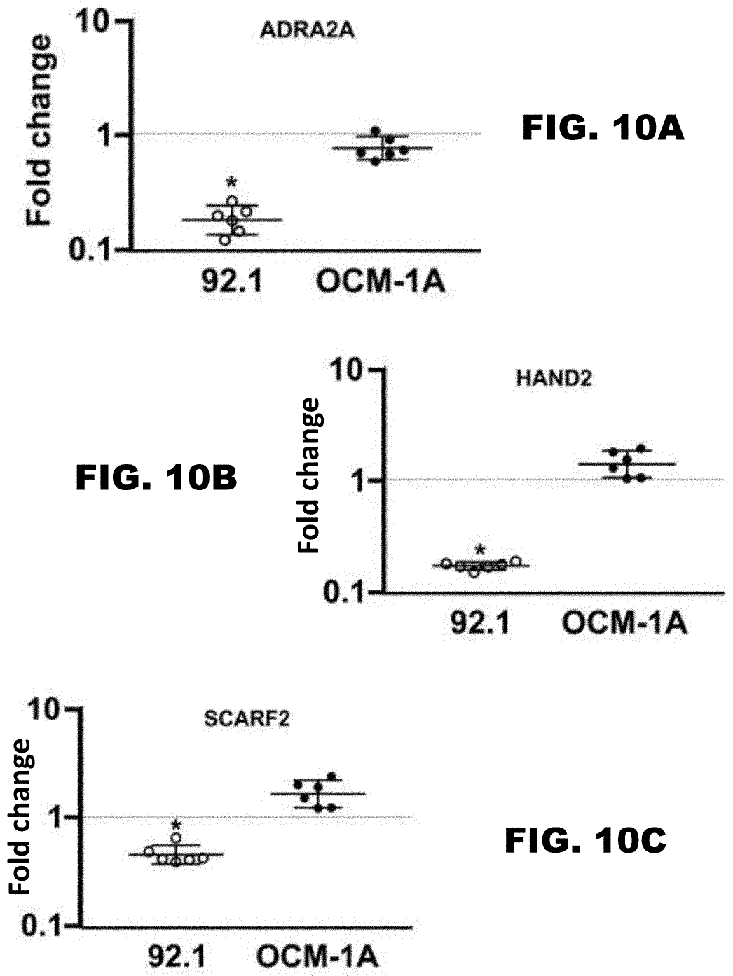

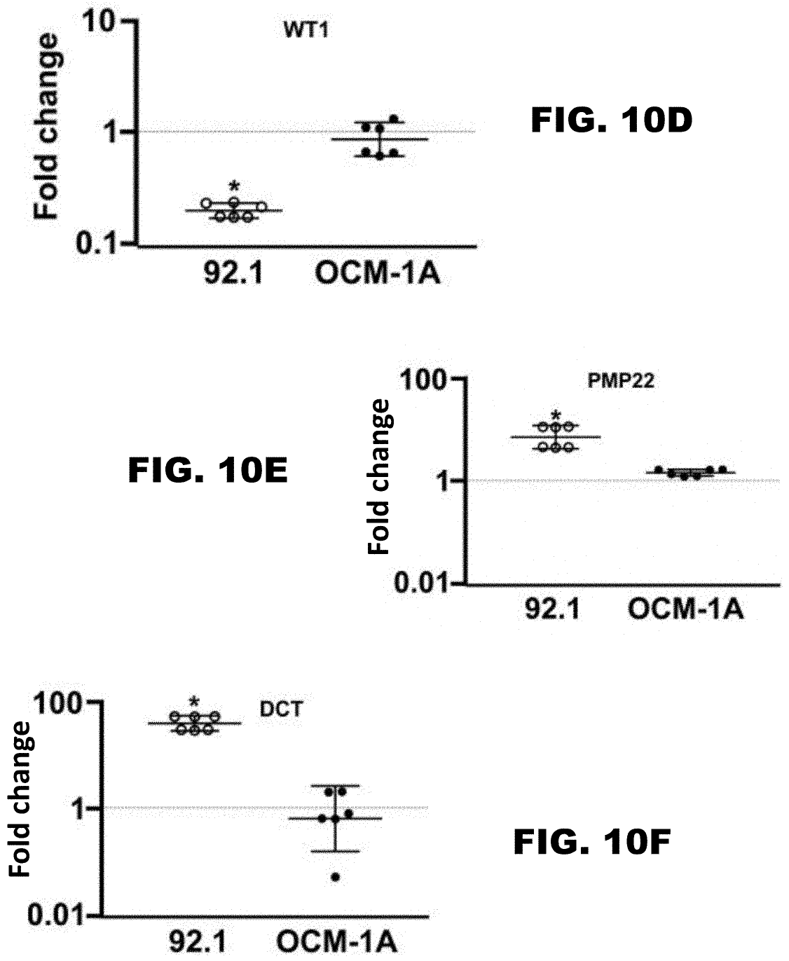

In contrast to the results above for apoptosis and cell cycle genes, large changes were observed for differentiation and developmental gene expression in FR-treated Gαq(Q209L)-driven 92.1 cells. Most strikingly, a distinct gene cluster was downregulated dramatically (4- to ˜160-fold; FIG. 7B; circled, and Table 3). Among genes in this cluster, 38% are associated with cell differentiation and development (FIG. 7C and Table 4), as revealed by Gene Ontology analysis. More important, 42% of genes in this cluster are known targets of the polycomb repressive complex 2 (PRC2) during differentiation from embryonic stem cells (FIG. 7D and Table 5), as indicated by gene set enrichment analysis. These results were confirmed by quantitative RT-PCR analysis of FR-treated Mel202 cells (FIG. 10). In contrast, expression of PRC2-regulated genes in BRAF(V600E)-driven OCM-1A cells was unaffected by FR (FIG. 10), demonstrating specificity of FR for developmental and differentiation genes targeted by constitutively active Gαq in UM cells

The RNA-seq analyses suggested a novel mechanism for Gαq-induced oncogenesis in UM in which constitutively active Gαq antagonizes PRC2-mediated gene repression, thereby reactivating genes associated with stemness and driving de-differentiation of UM cells into a more stem-like phenotype. FR treatment inhibits constitutively active Gαq, relieves blockade of PRC2-mediated repression, re-silences these genes, and returns UM cells to a melanocytic state. Consistent with this hypothesis, we found that inhibiting the catalytic subunit of PRC2 complexes (Ezh1/2) with GSK503 maintained 92.1 cells in an undifferentiated state and blocked the ability of FR to re-differentiate these cells as indicated by morphology (FIG. 7E) and pigmentation (FIG. 7F).

A striking and unanticipated consequence of inhibiting constitutively active Gαq in UM was discovered—the repression of gene sets that control differentiation and development. Many of these repressed genes are involved in embryonic stem cell lineage specification and differentiation, and they are targets of epigenetic silencing by the polycomb repressive complex 2 (PRC2), which acts through histone H3K27 trimethylation (34-36). These repressed genes include ADRA2A (alpha-adrenergic receptor-2A) and HAND2 (heart and neural crest derivatives expressed-2). The ADRA2A gene promoter was identified in independent screens for PRC2 subunit binding and for histone H3K27 trimethylation (34-36), and ADRA2A has been linked to cancer progression and severity (37). The HAND2 gene promoter is also targeted by PRC2 binding and histone H3K27 trimethylation (34-36), especially in migrating cranial neural crest cells, where HAND2 expression distinguishes neural crest cell lineages during facial development (38). Together, these findings reveal a novel mechanism in which signaling by constitutively active Gαq in UM cells antagonizes PRC2-mediated gene silencing, thereby maintaining UM cells in a less differentiated state similar to pre-melanocytic cranial neural crest cells (38).

### Materials and Methods for Examples 1-5

FR900359 was purified from A. crenata according to published methods (20). The structure of purified FR900359 relative to a commercially available equivalent (UBO-QIC; University of Bonn (Germany)) was established by NMR.

Biochemical Assays

Split luciferase assays were performed as described (24). The N-terminal portion of click beetle green luciferase (CBGN) was inserted into the αB-αC loop within the helical domain of wild type (WT) and constitutively active (Q/L) mutant forms of Gαq and Gα13. Insertion of foreign proteins at this site preserves Gα function (40). The C-terminal region of click beetle green luciferase (CBGC) was fused to the N-termini of Gβ1 (which was co-expressed with untagged Gγ2), RGS2, and the RGS domain of LARG, which interacts with Gα13 only in the active GTP-bound state (41). HEK-293 cells transiently transfected with various combinations of tagged proteins were treated 18 h with vehicle (DMSO) or FR and luciferase assays were performed to measure reconstituted luciferase activity.

Assays of transcriptional reporters driven by YAP or Gα subunits were performed as described (42, 43). YAP- or Gα-driven firefly luciferase activity was normalized to Renilla luciferase expressed from a constitutive promoter.

Experiments used to measure agonist-evoked inhibition of forskolin-induced cAMP formation in Neuro2A cells were performed as described (24). Cells were transfected with a cAMP FRET reporter and pertussis toxin (PTX)-resistant forms of Gαi1, Gαi/q or Gαi/q(R54K). After cells were treated 16 h with PTX to inactivate endogenously expressed Gi, FRET was used to measure inhibition of forskolin-induced cAMP formation by a CB1 cannabinoid receptor agonist (WIN 55,212-2; WIN).

Accumulation of IP1 in UM cells was measured using the IP-One kit (CiSbio, Inc; catalog number 62IPAPEB) according to the supplier's instructions. 10,000 Mel 202 cells, 20,000 92.1 cells and 20,000 OCM 1A cells were seeded into white-bottom tissue culture grade 384-well plates. Following an overnight incubation, cells were treated with FR or DMSO and returned to the incubator. The next day, stimulation buffer was added for 1 h, after which IP1-d2 and Ab-Cryp were added, and the cells were incubated at room temperature for 60 min. Plates were read in a Synergy H4 Hybred Reader (BioTek, Winooski, Vt., USA). Standard curves were generated using reagents supplied with the kit.

Cell Culture Assays

Cells were cultured at 37.0 in 5% CO2. Human 92.1, Mel202, and OCM-1A UM cells were derived by and the generous gifts of Drs. Martine Jager (Laboratory of Ophthalmology, Leiden University), Bruce Ksander (Schepens Eye Institute, Massachusetts Eye and Ear Infirmary) and June Kan-Mitchell (Biological Sciences, University of Texas at El Paso), respectively. Cell lines were grown in RPMI 1640 medium (Life Technologies, Carlsbad, Calif.) supplemented with 10% FBS and antibiotics. Cell viability was measured using a water-soluble tetrazolium salt, WTS-8 (Bimake, Houston, Tex.), following the manufacturer's protocol. Flow cytometry for analysis of cel proliferation and apoptosis was performed at the Siteman Cancer Center Flow Cytometry Core on a FACScan analyzer (BD Biosciences, San Diego Calif., USA) using a standard propidium iodide staining protocol as described previously (44).

Immunostaining

Cell fixation was carried out by adding an equal volume of 2× fixative (PBS with 4% paraformaldehyde and 0.4% glutaraldehyde) to UM cells in RPMI growth medium. After 15 min at 37° C., cells were permeabilized with 0.1% Triton X-100 in PBS for 5 min, washed with PBS, and blocked with 2% fish gelatin (Sigma-Aldrich) in PBS. Primary and secondary antibodies were diluted in 2% fish gelatin in PBS. Primary antibodies included mouse monoclonal anti-pre-melanosomal protein (One World Lab, San Diego, Calif.), rabbit polyclonal anti-tyrosinase (One World Lab), rabbit polyclonal anti-dopachrome tautomerase (One World Lab), rabbit polyclonal anti-S100 (DakoCytomation, Denmark), and mouse monoclonal anti-BrdU (Life Technologies). Secondary antibodies were Alexa-fluor conjugates (Life Technologies), and the mounting agent was ProLong Gold (Life Technologies). Cell morphology was assessed by phase contrast imaging with an inverted microscope (Olympus IX72) using a 10× objective.

Immunoblotting

For standard immunoblots, cells were lysed and cleared in radioimmunoprecipitation assay buffer (150 mM sodium chloride, 1% Triton-X100, 0.5% sodium deoxycholate, 0.1% sodium dodecyl sulfate, 50 mM Tris, pH 8.0) with 1× Complete Protease Inhibitor (Roche, cat.11697498001). Histones were isolated from cells using the Active Motif Histone Purification Mini Kit (cat.40026, Active Motif, Carlsbad, Calif.). Lysates were resolved on 15% SDS-PAGE gels and transferred to Immobilon (P) PVDF membrane (Milipore, cat.IPVH00010). Membranes were blocked with 5% w/v milk in TBST (25 mM Tris pH 7.2, NaCl 150 mM, 2.7 mM KCl, 0.1% v/v Tween 20) and incubated with primary antibodies. Membranes were washed with TBST at least three times and incubated with IRDye 680 Goat anti-rabbit and IRDye 800 Goat anti-mouse (LI-COR, Lincoln, Nebr.). Following incubation, membranes were washed at least three times with TBST and signals were detected using LI-COR Odyssey model 9120 imaging system (LI-COR). Primary antibodies used for immunoblots were: anti-EE (Covance, cat. MMS-115P, lot E12BF00285), anti-Actin C4 (Millipore, cat. MAB1501), anti-Histone H3 (clone A3S, cat.05-928, Millipore) and Anti-histone H3-trimethyl-K27 (cat.6002, Abcam).

Gene Expression Analysis

92.1 UM cells were treated with 100 nM FR or vehicle (DMSO) in RPMI growth medium and collected after 1 and 3 d of treatment. RNA was isolated using the RNeasy Mini Kit (Qiagen) following the manufacturer's protocol and including the optional DNase I treatment step. RNA quality was assessed on a Bioanalyzer 2100 (Agilent Technologies, Santa Clara, Calif., USA). mRNA was extracted from total RNA using a Dynal mRNA Direct kit, fragmented and reverse transcribed to double stranded cDNA with random primers before addition of adapters for library preparation. Library preparation and HiSeq2500 sequencing were performed by the Washington University Genome Technology Access Center (gtac.wustl.edu). FastQ files were aligned to the transcriptome and the whole-genome with STAR. Biologic replicates were simultaneously analyzed by edgeR and Sailfish analyses of gene-level/exon-level features. Unexpressed genes and exons were removed from the analyses. Unsupervised principal component analysis and volcano plots were generated in Bioconductor using edgeR. Significance Analysis of Microarrays (SAM), Version 4.0 was used to generate a ranked gene list, and a threshold of q<10% and fold-change>2.0 was then used to select the most highly significant genes that were down regulated in FR-treated versus vehicle control cells. This list was used as signature gene sets for Gene Ontology (GO) analysis and Gene Set Enrichment Analysis (GSEA) (45). GO groups were assembled by merging the lists of genes from related GO terms that were significantly enriched in the signature gene set. Significant gene sets from GSEA analyses were combined such that genes associated with multiple related signatures were only counted once and each gene was assigned only to a single combined groupbased on the signature with the highest enrichment score for that gene. Significant gene expression changes were validated in all three UM cell lines by qPCR using fast SYBR Green Mastermix (Fisher Scientific) following the manufacturers' protocol. GAPDH was used as an endogenous control. Primer sets used for the assay are listed in table 6.

TABLE 2
Gene Set Enrichment Negative Correlation

| | | | | NOM | FDR | FWER | RANK |
|---|---|---|---|---|---|---|---|
| NAME | SIZE | ES | NES | p-val | [symbol] | p-val | AT MAX |
| KEGG_RIBOSOME | 77 | [symbol] | −4.11456 | 0 | 0 | 0 | 1165 |
| REACTOME_PEPTIDE_CHAIN_ELONGATION | 78 | −0.8362932 | −4.040971 | 0 | 0 | 0 | 1165 |
| REACTOME_3_UTR_MEDIATED_TRANSLATIONAL_ | 93 | −0.7894343 | −4.0002007 | 0 | 0 | 0 | 806 |
| REGULATION | | | | | | | |
| REACTOME_INFLUENZA_VIRAL_RNA_ | 89 | −0.7629774 | −3.777758 | 0 | 0 | 0 | 1414 |
| TRANSCRIPTIONAL_REGUALTION | | | | | | | |
| REACTOME_NONSENSE_MEDIATED_DECAY_ | 90 | −0.7575414 | −3.775522 | 0 | 0 | 0 | 806 |
| ENHANCED_BY_THE_EXON_JUNCTION_COMPLEX | | | | | | | |
| REACTOME_SRP_DEPENDENT_COTRANSLATIONAL_ | 88 | −0.7586833 | −3.77252 | 0 | 0 | 0 | 1165 |
| PROTEIN_TARGETING_TO_MEMBRANE | | | | | | | |
| [symbol] _SERUM_AND_RAPAMYCIN_SENSITIVE_ | 56 | −0.8275592 | −3.744492 | 0 | 0 | 0 | 890 |
| GENES | | | | | | | |
| REACTOME_TRANSLATION | 117 | −0.5966806 | −3.614177 | 0 | 0 | 0 | 1222 |
| REACTOME_FORMATION_OF_THE_TERNARY_ | 42 | −0.7636944 | −3.333641 | 0 | 0 | 0 | 1414 |
| COMPLEX_AND_SUBSEQUENTLY_THIS_43S_ | | | | | | | |
| COMPLEX | | | | | | | |
| [symbol] _ARREST_BY_ [symbol] _DN | 69 | −0.6813395 | −3.252417 | 0 | 0 | 0 | 1562 |
| [symbol] _MULTIPLE_MYELOMA_HYPERLOID_UP | 40 | −0.7651164 | −3.199398 | 0 | 0 | 0 | 1320 |
| [symbol] _AMINO_ACID_DEPRIVATION | 24 | −0.8503655 | −3.140422 | 0 | 0 | 0 | 1094 |
| REACTOME_ACTIVATION_OF_THE_MRNA_UPON_ | 48 | −0.725141 | −3.124917 | 0 | 0 | 0 | 1414 |
| BINDING_OF_THE_CAP_BINDING_COMPLEX_ | | | | | | | |
| AND_ [symbol] _AND_SUBSEQUENT_BINDING_ | | | | | | | |
| UO_ [symbol] | | | | | | | |
| ALK_DN, V1_UP | 56 | −0.6754234 | −3.122742 | 0 | 0 | 0 | 985 |
| [symbol] _SERUM_RESPONSE_TRANSLATION | 25 | −0.7377167 | −3.031653 | 0 | 0 | 0 | 875 |
| REACTOME_INFLUENZA_LIFE_CYCLE | 117 | −0.5825156 | −3.005197 | 0 | 0 | 0 | 806 |
| REACTOME_METABOLISM_OF_MRNA | 150 | −0.5385245 | −2.898579 | 0 | 0 | 0 | 806 |
| [symbol] _MAMMARY_STEM_CELL_UP | 89 | −0.5578858 | −2.803973 | 0 | 0 | 0 | 1222 |
| ZHANG_ [symbol] _TARGETS_UP | 64 | −0.5840745 | −2.797299 | 0 | 0 | 0 | 1627 |
| [symbol] _PEDIATRIC_ [symbol] _THERAPY_ | 39 | −0.6667184 | −2.788914 | 0 | 0 | 0 | 1090 |
| RESPONSE_UP | | | | | | | |
| [symbol] _INTESTINE_PROBIOTICS_24HR_DN | 160 | −0.483478 | −2.683889 | 0 | 0 | 0 | 1198 |
| [symbol] _INNER_ [symbol] | 27 | −0.7179842 | −2.671325 | 0 | 0 | 0 | 1098 |
| [symbol] _TP53_MRAS_COOPERATION_RESPONSE_DN | 31 | −0.5834503 | −2.650262 | 0 | 0 | 0 | 1285 |
| PENG_LEUCINE_DEPRIVATION_UP | 100 | −0.5277748 | −2.629929 | 0 | 0 | 0 | 1633 |
| [symbol] _GENOTOXIC_DAMAGE_24HR | 24 | −0.7261035 | −2.629237 | 0 | 0 | 0 | 834 |
| ZHAN_MULTIPLE_MYELOMA_CD1_VS_CD2_UP | 44 | −0.5992472 | −2.602829 | 0 | 0 | 0 | 1256 |
| REACTOME_METABOLISM_OF_RNA | 180 | −0.5440802 | −2.581455 | 0 | 0 | 0 | 806 |
| [symbol] _TARGETS_OF_IGF1_AND_IGF2_UP | 27 | −0.679906 | −2.571272 | 0 | 0 | 0 | 881 |
| [symbol] _RESPONSE_TO_ [symbol] _UP | 35 | −0.6332173 | −2.557885 | 0 | 0 | 0 | 837 |
| PODAR_RESPONSE_TO_ADAPHOSTIN_UP | 104 | −0.5083627 | −2.557086 | 0 | 0 | 0 | 1633 |
| [symbol] _PLASMA_CELL_VS_PLASMABLAST_UP | 184 | −0.4464259 | −2.5482 | 0 | 0 | 0 | 1390 |
| [symbol] _SICKLE_CELL_DISEASE_DN | 116 | −0.4751957 | −2.531408 | 0 | 0 | 0 | 875 |
| GAUSSMANN_MLL_ [symbol] _FUSION_TARGETS_F_UP | 106 | −0.4918961 | −2.5305 | 0 | 0 | 0 | 1582 |
| [symbol] _INTESTINE_PROBIOTICS_6HR_UP | 47 | −0.5729876 | −2.518115 | 0 | 0 | 0 | 733 |
| [symbol] _TARGETS_UP | 106 | −0.4853156 | −2.514798 | 0 | 0 | 0 | 2047 |
| [symbol] _NEURAL_CREST_STEM_CELL_DN | 60 | −0.5112373 | −2.510382 | 0 | 0 | 0 | 1537 |
| [symbol] _RESPONSE_TO_ [symbol] _UP | 16 | −0.7423251 | −2.491617 | 0 | 0 | 0 | 1429 |
| MIKKELSEN_MCV6_HCP_WITH_ [symbol] | 139 | −0.4619398 | −2.475651 | 0 | 0 | 0 | 1429 |
| [symbol] _ASPARAGINAS_RESISTANCE_ [symbol] _UP | 19 | −0.7230168 | −2.468087 | 0 | 0 | 0 | 1222 |
| KRAS.LUNG.BREAST_UP, V1_DN | 40 | −0.5628054 | −2.3475 | 0 | 0 | 0 | 1983 |
| [symbol] _UP, V1_DN | 63 | −0.5127742 | −2.342012 | 0 | 0 | 0 | 1872 |
| YAO_TEMPORAL_RESPONSE_TO_PROGESTERONE_ | 47 | −0.5619978 | −2.458571 | 0 | 4.80E−05 | 0.002 | 1426 |
| CLUSTER_O | | | | | | | |
| [symbol] _RESPONSE_TO_ [symbol] _UP | 28 | −0.6260312 | −2.4359 | 0 | 6.93E−05 | 0.003 | 471 |
| [symbol] _RESPONSE_TO_ [symbol] _UP | 158 | −0.4429056 | −2.428607 | 0 | 9.01E−05 | 0.004 | 1719 |
| POMERPY_MEDULOBLASTOMA_DESMOPLASIC_VS_ | 31 | −0.6005085 | −2.387366 | 0 | 9.65E−05 | 0.005 | 911 |
| CLASSIC_DN | | | | | | | |
| REACTOME_CYTOSOLIC_TRNA_AMINOACYLATION | 19 | −0.6974551 | −2.393741 | 0 | 9.85E−05 | 0.005 | 721 |
| [symbol] _HOUSEKEEPING_GENES | 279 | −0.398129 | −2.395038 | 0 | 1.01E−04 | 0.005 | 1367 |
| ONDONNELL_TARGETS_OF_MYC_AND_TFRC_UP | 47 | −0.5322069 | −2.403383 | 0 | 1.05E−04 | 0.004 | 1390 |
| [symbol] _TARGETS_UP | 93 | −0.4737549 | −2.403383 | 0 | 1.05E−04 | 0.005 | 1518 |
| MIKKELSEN_ [symbol] | 103 | −0.4727085 | −2.507248 | 0 | 1.08E−04 | 0.005 | 1886 |
| [symbol] _DEGRADED_VIA_ [symbol] | 43 | −0.5497911 | −2.408297 | 0 | 1.10E−04 | 0.005 | 1303 |
| [symbol] | 410 | −0.381772 | −2.371255 | 0 | 1.27E−04 | 0.007 | 1922 |
| [symbol] _SILENCED_BY_METHYLATION_DN | 69 | −0.4980554 | −2.3798 | 0 | 1.30E−04 | 0.007 | 1298 |
| [symbol] _FLAVL1_TARGETS_UP | 79 | −0.49801521 | −2.383254 | 0 | 1.32E−04 | 0.007 | 817 |
| KOBAYASHI_EGFR_SIGNALING_24HR_UP | 58 | −0.5187423 | −2.367372 | 0 | 1.43E−04 | 0.008 | 1592 |
| [symbol] _TP53_TARGETS | 21 | −0.5636328 | −2.34865 | 0 | 1.56E−04 | 0.009 | 1584 |
| MARTENS_TRETINOIN_RESPONSE_UP | 237 | −0.4094868 | −2.352606 | 0 | 1.59E−04 | 0.009 | 1471 |
| [symbol] | 31 | −0.5890119 | −2.34034 | 0 | 2.20E−04 | 0.013 | 1225 |
| KASLER_HDAC7_TARGETS_2_DN | 21 | −0.6502378 | −2.320462 | 0 | 2.23E−04 | 0.016 | 802 |
| WEST_ADRENOCORTICAL_TUMOR_DN | 261 | −0.3968828 | −2.322684 | 0 | 2.78E−04 | 0.016 | 1262 |
| [symbol] _PRC2_TARGETS | 232 | −0.4077365 | −2.323629 | 0 | 2.83E−04 | 0.016 | 1922 |
| MIKKELSEN_ [symbol] _WITH_ [symbol] | 81 | −0.5719433 | −2.286906 | 0 | 4.09E−04 | 0.025 | 1655 |
| OSMAN_BLADDER_CANCER_DN | 220 | −0.3983562 | −2.286096 | 0 | 4.18E−04 | 0.026 | 1356 |
| [symbol] _BMP2_TARGETS_UP | 367 | −0.3741874 | −2.283134 | 0 | 4.26E−04 | 0.027 | 1707 |
| [symbol] _BRAIN_HCP_WITH_ [symbol] | 91 | −0.4481213 | −2.25689 | 0 | 5.22E−04 | 0.035 | 1852 |
| [symbol] _MULTIPLE_MYELOMA_UP | 29 | −0.5666021 | −2.253276 | 0 | 5.28E−04 | 0.035 | 717 |
| [symbol] _THYROID_CANCER_DN | 118 | −0.4291295 | −2.252387 | 0 | 5.30E−04 | 0.035 | 1760 |
| MIKKELSEN_ [symbol] _WITH_ [symbol] | 146 | −0.4149404 | −2.264092 | 0 | 5.39E−04 | 0.035 | 1688 |
| [symbol] | 68 | −0.44882245 | −2.12524 | 0 | 6.14E−04 | 0.002 | 1515 |
| [symbol] _TARGETS_UP | 21 | −0.6239096 | −2.233306 | 0 | 6.92E−04 | 0.048 | 1445 |
| [symbol] _RESPONSE_TO_METHOTREXATE_UP | 17 | −0.6758122 | −2.233308 | 0 | 7.02E−04 | 0.048 | 1314 |
| [symbol] _DN, V1_UP | 55 | −0.4773785 | −2.129865 | 0 | 7.37E−04 | 0.002 | 1859 |
| [symbol] _TARGETS_UP | 21 | −0.5318697 | −2.222122 | 0 | 7.49E−04 | 0.053 | 1748 |
| [symbol] | 129 | −0.4086753 | −2.220779 | 0 | 1.65E−04 | 0.055 | 1700 |
| KRAS [symbol] | 65 | −0.4222526 | −2.084648 | 0 | 7.86E−04 | 0.008 | 1867 |
| CHANG_CORE_SERUM_RESPONSE_DN | 125 | −0.4167168 | −2.209614 | 0 | 8.19E−04 | 0.059 | 1534 |
| ZHAN_MULTIPLE_MYELOMA_CD1_UP | 26 | −0.5883222 | −2.202299 | 0 | 8.22E−04 | 0.061 | 1094 |
| GENTILE_UV_LOW_DOSE_UP | 17 | −0.6674533 | −2.20004 | 0 | 8.23E−04 | 0.062 | 1462 |
| [symbol] _SILENCED_DURING_TUMOR_ANGIOGENESIS | 32 | −0.5556766 | −2.204214 | 0 | 8.34E−04 | 0.061 | 1098 |
| RICKMAN_HEAD_AND_NECK_CANCER_ [symbol] | 15 | −0.6841369 | −2.204813 | 0 | 8.46E−04 | 0.061 | 2000 |
| [symbol] | 54 | −0.4576916 | −2.084719 | 0 | 8.84E−04 | 0.004 | 1678 |
| [symbol] | 75 | −0.4348109 | −2.336282 | 0 | 9.21E−04 | 0.002 | 1508 |
| [symbol] _SOFT_TISSUE_TUMORS_PCA1_DN | 35 | −0.5231197 | −2.180453 | 0 | 9.84E−04 | 0.078 | 1867 |
| REACTOME_AMINO_ACID_TRANSPORT_ACROSS_THE_ | 20 | −0.6054498 | −2.182732 | 0 | 9.85E−04 | 0.076 | 992 |
| PLASMA_MEMBRANE | | | | | | | |
| ZHAN_MULTIPLE_MYELOMA_HP_UP | 28 | −0.5562334 | −2.17634 | 0 | 9.93E−04 | 0.081 | 1465 |
| [symbol] _TARGETS_OF_ [symbol] | 192 | −0.3867057 | −2.179024 | 0 | 9.94E−04 | 0.08 | 1687 |
| [symbol] _TARGETS | 393 | −0.3540429 | −2.181025 | 0 | 5.97E−04 | 0.078 | 1922 |
| MTOR_UP [symbol] | 89 | −0.4243262 | −2.0919 | 0 | 0.0010103 | 0.004 | 2035 |
| GARGALOVIC_RESPONSE_TO_OXIDIZED_ | 15 | −0.6794893 | −2.155882 | 0 | 0.00114316 | 0.095 | 1394 |
| PHOSPHOLIPIDS_RED_UP | | | | | | | |
| [symbol] _RESPONSE_TO_CISPLATIN | 15 | [symbol] | −2.160356 | 0 | 0.0012011 | 0.102 | 1314 |
| [symbol] _LIVER_CANCER_SUBCLASS_G3_DN | 26 | −0.5759184 | −2.162662 | 0 | 0.00120454 | 0.101 | 1534 |
| [symbol] _STEM_CELL_CULTURED_VS_FRESH_UP | 246 | [symbol] | [symbol] | 0 | 0.00120818 | 0.101 | 1120 |
| ZHANG_ [symbol] _TARGETS_60HR_UP | 170 | [symbol] | [symbol] | 0 | 0.00126072 | 0.109 | 1464 |
| CHO_ [symbol] _TARGETS | 18 | [symbol] | [symbol] | 0 | [symbol] | 0.109 | 985 |
| MODY_HIPPOCAMPUS_PRENATAL | 30 | [symbol] | [symbol] | 0 | [symbol] | 0.135 | [symbol] |
| [symbol] _PROSTATE_CARCINOGENESIS_UP | 37 | −0.5153462 | [symbol] | 0 | [symbol] | 0.159 | 442 |
| [symbol] | 64 | [symbol] | [symbol] | 0 | [symbol] | 0.011 | 1747 |
| [symbol] _METHYLATED_DE_NOVO_IN_CANCER | 34 | [symbol] | [symbol] | 0 | [symbol] | 0.184 | 2277 |
| [symbol] _METASTASIS_DN | 88 | [symbol] | [symbol] | 0 | [symbol] | 0.19 | 2181 |
| MAN_INK_SIGNALING_DN | 20 | [symbol] | [symbol] | 0 | [symbol] | 0.199 | [symbol] |
| ACEVEDO_ [symbol] _TARGETS_IN_PROSTATE_ | 142 | [symbol] | [symbol] | 0 | [symbol] | 0.204 | 1676 |
| CANCER_MODEL_DN | | | | | | | |
| ODONNELL_TFRC_TARGETS_UP | 196 | [symbol] | [symbol] | 0 | [symbol] | 0.223 | 1526 |
| [symbol] _TARGETS_DN | 31 | [symbol] | [symbol] | 0 | [symbol] | 0.237 | 1008 |
| [symbol] _DN_ [symbol] | 68 | [symbol] | [symbol] | 0 | [symbol] | 0.017 | 208 |
| [symbol] _SECRETED_FACTERS | 75 | −0.4331841 | −2.08575 | 0 | [symbol] | 0.26 | 2139 |
| [symbol] | 36 | −0.4718207 | [symbol] | 0 | [symbol] | 0.02 | 2016 |
| [symbol] | 74 | [symbol] | [symbol] | 0 | [symbol] | 0.024 | 1582 |
| [symbol] _RESPONSE_TO_ [symbol] | 31 | [symbol] | [symbol] | 0 | [symbol] | 0.272 | 844 |
| KRAS.KIDNEY_ [symbol] | 67 | [symbol] | [symbol] | 0 | [symbol] | 0.023 | 1926 |
| KRAS.BREAST_ [symbol] | 47 | [symbol] | [symbol] | 0 | [symbol] | 0.027 | 1983 |
| [symbol] BAUER_MYOGENIC_TARGETS_OF_AX3_ | 23 | [symbol] | [symbol] | 0 | [symbol] | 0.298 | 183 |
| FOXO1_FUSION | | | | | | | |
| REACTOME_METABOLISM_OF_PROTEINS | 281 | [symbol] | [symbol] | 0 | [symbol] | 0.333 | 902 |
| [symbol] _RESPONSE_TO_CISPLATIN_UP | 25 | [symbol] | [symbol] | 0 | [symbol] | 0.341 | 1012 |
| [symbol] | 80 | [symbol] | [symbol] | 0 | [symbol] | 0.039 | 1582 |
| [symbol] _TARGETS | 407 | −0.3305 | −2.04566 | 0 | [symbol] | 0.352 | 1922 |
| [symbol] _GASTRIC_CANCER_EARLY_DN | 138 | [symbol] | [symbol] | 0 | [symbol] | 0.363 | 1683 |
| [symbol] _PILOCYTIC_ASTROCYTOMA_VS_ | 23 | [symbol] | [symbol] | 0 | [symbol] | 0.368 | 1004 |
| GLIOBLASTOME_UP | | | | | | | |
| KIM_RESPONSE_TO_TSA_AND_DECITABINE_UP | 47 | [symbol] | [symbol] | 0 | [symbol] | 0.385 | 1745 |
| NABA_MATRISOME | 332 | [symbol] | [symbol] | 0 | [symbol] | 0.392 | 1622 |
| [symbol] _TARGETS_DN | [symbol] | [symbol] | [symbol] | 0 | [symbol] | 0.392 | 1683 |
| [symbol] _RESPONSE_SGY | 24 | [symbol] | [symbol] | 0 | [symbol] | 0.4 | 1278 |
| CHANDRAN_METASTASIS_TOP50_DN | 33 | [symbol] | [symbol] | 0 | [symbol] | 0.417 | 1145 |
| [symbol] _EPITHELIAL_MESENCHYMAL_TRANSITION_ | 85 | [symbol] | [symbol] | 0 | [symbol] | 0.45 | 1663 |
| DN | | | | | | | |
| NAKAMURA_TUMOR_ZONE_PERIPHERAL_VS_CENTRAL_ | 359 | [symbol] | [symbol] | 0 | [symbol] | 0.467 | 1642 |
| DN | | | | | | | |
| [symbol] _ADIPOGENIC_POTENTIAL_DN | 22 | [symbol] | [symbol] | 0 | [symbol] | 0.52 | 1152 |
| [symbol] _CHRONIC_MEYLOGENOUS_LEUKEMIA_DN | [symbol] | [symbol] | [symbol] | 0 | [symbol] | 0.547 | 1738 |
| [symbol] _TARGETS_DN | 79 | [symbol] | [symbol] | 0 | [symbol] | 0.564 | 1335 |
| SENESE_ [symbol] _TARGETS_DN | 130 | [symbol] | [symbol] | 0 | [symbol] | 0.564 | 1581 |
| [symbol] _BREAST_4_ [symbol] _UP | 125 | [symbol] | [symbol] | 0 | [symbol] | 0.571 | [symbol] |
| [symbol] _AMPLIFIED_IN_LUNG_CANCER | 107 | [symbol] | [symbol] | 0 | [symbol] | 0.583 | 1342 |
| KEGG_GLYCINE_SERINE_AND_THREONINE_ | 16 | [symbol] | [symbol] | 0 | [symbol] | 0.612 | 1228 |
| METABOLISM | | | | | | | |
| [symbol] | 86 | [symbol] | [symbol] | 0 | [symbol] | 0.079 | 1874 |
| [symbol] _SILENCED_BY_METHYLATION_IN_ | 22 | [symbol] | [symbol] | 0 | [symbol] | 0.654 | 729 |
| PANCREATIC_CANCER_2 | | | | | | | |
| WANG_HCP_PROSTATE_CANCER | 67 | [symbol] | [symbol] | 0 | [symbol] | 0.662 | 1557 |
| [symbol] _LIVER_CANCER_WITH_EPCAM_UP | 43 | [symbol] | [symbol] | 0 | [symbol] | 0.672 | [symbol] |
| [symbol] _MELANOMA_DN | 357 | [symbol] | [symbol] | 0 | [symbol] | 0.698 | 1090 |
| [symbol] | 80 | [symbol] | [symbol] | 0 | [symbol] | 0.101 | 1215 |
| [symbol] _TARGETS | 42 | [symbol] | [symbol] | 0 | [symbol] | 0.712 | 834 |
| [symbol] _TARGETS_DN | 57 | [symbol] | [symbol] | 0 | [symbol] | 0.731 | 1537 |
| [symbol] _TARGETS_OF_ [symbol] _FUSION_ [symbol] | 67 | [symbol] | [symbol] | 0 | [symbol] | 0.729 | 1616 |
| [symbol] _SILENCED_BY_METHYLATION_IN_ | 178 | [symbol] | [symbol] | 0 | [symbol] | 0.741 | 870 |
| PANCREATIC_CANCER_1 | | | | | | | |
| WANG_RESPONSE_TO_ [symbol] _INHIBITOR_ [symbol] | 173 | [symbol] | [symbol] | 0 | [symbol] | 0.743 | 1476 |
| [symbol] _RESPONSE_TO_ [symbol] _24HR_UP | 437 | [symbol] | [symbol] | 0 | [symbol] | 0.762 | 1656 |
| [symbol] _INTESTINE_PROBIOTICS_2HR_UP | 20 | [symbol] | [symbol] | 0 | [symbol] | 0.776 | 623 |
| [symbol] _MATRISOME | 106 | [symbol] | [symbol] | 0 | [symbol] | 0.783 | 1995 |
| [symbol] | 60 | [symbol] | [symbol] | 0 | [symbol] | 0.13 | 1456 |
| MIKKELSEN_NPC_HCP_WITH_ [symbol] | 113 | [symbol] | [symbol] | 0 | [symbol] | 0.791 | 1236 |
| ZWANG_EGF_INTERVAL_DN | 105 | [symbol] | [symbol] | 0 | [symbol] | 0.797 | 1574 |
| [symbol] | 115 | [symbol] | [symbol] | 0 | [symbol] | 0.129 | 1252 |
| [symbol] | 99 | [symbol] | [symbol] | 0 | [symbol] | 0.158 | 2255 |
| [symbol] | 65 | [symbol] | [symbol] | 0 | [symbol] | 0.154 | [symbol] |
| [symbol] | 98 | [symbol] | [symbol] | 0 | [symbol] | 0.147 | 1926 |
| ZHANG_ [symbol] _TARGETS_36HR_UP | [symbol] | [symbol] | [symbol] | 0 | [symbol] | [symbol] | 1627 |
| [symbol] _BREAST_CANCER_BASAL_DN | 329 | [symbol] | [symbol] | 0 | [symbol] | 0.87 | 1675 |
| [symbol] _STEM_CELL_UP | 120 | [symbol] | [symbol] | 0 | [symbol] | 0.879 | 1098 |
| [symbol] _BREAST_CANCER_LUMINAL_VS_ | 207 | [symbol] | [symbol] | 0 | [symbol] | 0.879 | 2112 |
| MESENCHYMAL_UP | | | | | | | |
| [symbol] _RESPONSE_TO_LEUKOTRIENE_AND_ | 20 | [symbol] | [symbol] | 0 | [symbol] | 0.895 | 1342 |
| THROMBIN | | | | | | | |
| [symbol] _DLBCL_VS_FOLLICULAR_LYMPHOMA_DN | 19 | [symbol] | [symbol] | 0 | [symbol] | 0.898 | 829 |
| RODRIGUES_THYROID_CARCINOMA_ANAPLASTIC_DN | 292 | [symbol] | [symbol] | 0 | [symbol] | 0.915 | 1225 |
| [symbol] _TARGETS_SILENCED_BY_METHYLATION_DN | 156 | [symbol] | [symbol] | 0 | [symbol] | [symbol] | 1722 |
| [symbol] _TP53_TARGETS_UP | 30 | [symbol] | [symbol] | 0 | [symbol] | 0.925 | 1234 |
| [symbol] _LVAD_SUPPORT_OF_FAILING_HEART_UP | 61 | [symbol] | [symbol] | 0 | [symbol] | 0.923 | 1953 |
| SENESE_HDAC2_TARGETS_DN | 58 | [symbol] | [symbol] | 0 | [symbol] | 0.927 | 1265 |
| [symbol] _THYROID_CANCER_CLUSTER_3 | 18 | [symbol] | [symbol] | 0 | [symbol] | 0.945 | 1571 |
| [symbol] _CDH1_TARGETS_2_DN | 205 | [symbol] | [symbol] | 0 | [symbol] | [symbol] | 1698 |
| [symbol] _MATRISONE_ASSOCIATED | 226 | [symbol] | [symbol] | 0 | [symbol] | 0.953 | 1622 |
| GROSS_HYPOXIA_VIA_ELK3_DN | 91 | [symbol] | [symbol] | 0 | [symbol] | 0.958 | 1196 |
| [symbol] _BREAST_CARCINOMA_METAPLASTIC_VS_ | 39 | [symbol] | [symbol] | 0 | [symbol] | 0.973 | 1554 |
| DUCTAL_DN | | | | | | | |
| KEGG_AMINOACYL_TRNA_BIOSYNTHESIS | 31 | [symbol] | [symbol] | 0 | [symbol] | 0.575 | [symbol] |
| [symbol] _MULTIPLE_MYELOMA_PCA1-_UP | 36 | [symbol] | [symbol] | 0 | [symbol] | [symbol] | 1120 |
| [symbol] _ACUTE_PROMYELOCYTIC_LEUKEMIA_UP | 84 | [symbol] | [symbol] | 0 | [symbol] | 0.981 | 1820 |
| KAN_RESPONSE_TO_ARSENIC_ [symbol] | 77 | [symbol] | [symbol] | 0 | [symbol] | 0.992 | 1177 |
| TANG_SENESCENCE_TP53_TARGETS_UP | 19 | [symbol] | [symbol] | 0 | [symbol] | 0.992 | [symbol] |
| [symbol] | [symbol] | [symbol] | [symbol] | 0 | [symbol] | 0.022 | 1780 |
| [symbol] _TNF_TARGETS-_UP | 31 | [symbol] | [symbol] | 0 | [symbol] | 0.994 | 1760 |
| CHEN_HOXA5_TARGETS_9HR_UP | 119 | [symbol] | [symbol] | 0 | [symbol] | 0.994 | 1862 |
| WANG_SMARCI1_TARGETS_UP | 145 | [symbol] | [symbol] | 0 | [symbol] | 0.996 | [symbol] |
| [symbol] _APOPTOSIS_BY_EPOXOMICIN_UP | 158 | [symbol] | [symbol] | 0 | [symbol] | 0.997 | 1458 |
| [symbol] _MMP14_TARGETS_UP | 134 | [symbol] | [symbol] | 0 | [symbol] | 0.997 | 1621 |
| OCT1_01 | 106 | [symbol] | [symbol] | 0 | [symbol] | 0.166 | 1738 |
| [symbol] _APOPTOSIS_VIA_TRAIL_DN | 99 | [symbol] | [symbol] | 0 | [symbol] | 0.997 | 1511 |
| [symbol] _HEPATOBLASTOMA_CLASSES_DN | 103 | [symbol] | [symbol] | 0 | [symbol] | 0.998 | 1338 |
| [symbol] | 75 | [symbol] | [symbol] | 0 | [symbol] | 0.998 | 1119 |
| CHOP_01 | 128 | [symbol] | [symbol] | 0 | [symbol] | 0.155 | 1747 |
| [symbol] | 57 | [symbol] | [symbol] | 0 | [symbol] | 0.435 | 1687 |
| [symbol] | 105 | [symbol] | [symbol] | 0 | [symbol] | 0.133 | 1777 |
| [symbol] | 114 | [symbol] | [symbol] | 0 | [symbol] | 0.291 | 1489 |
| [symbol] | 113 | [symbol] | [symbol] | 0 | [symbol] | 0.107 | 1415 |
| [symbol] _FUSION_TARGETS_A_DN | 58 | [symbol] | [symbol] | 0 | [symbol] | 0.998 | 1210 |
| [symbol] _STROMA_S_UP | 152 | [symbol] | [symbol] | 0 | [symbol] | 0.998 | 2007 |
| SCHAEFFER_PROSTATE_DEVELOPMENT_48HR_DN | 195 | [symbol] | [symbol] | 0 | [symbol] | 0.998 | 1537 |
| KARLSSON_TGFB1_TARGETS_DN | 130 | [symbol] | [symbol] | 0 | [symbol] | 0.998 | 1387 |
| [symbol] | 45 | [symbol] | [symbol] | 0 | [symbol] | 0.282 | 1822 |
| [symbol] | 112 | [symbol] | [symbol] | 0 | [symbol] | 0.238 | 1496 |
| [symbol] | 108 | [symbol] | [symbol] | 0 | [symbol] | 0.279 | 1097 |
| [symbol] _BREAST_DUCTAL_CARCINOMA_VS_ | 78 | [symbol] | [symbol] | 0 | [symbol] | 0.999 | 2141 |
| DUCTAL_NORMAL_DN | | | | | | | |
| [symbol] _GLYCOPROTEINS | 78 | [symbol] | [symbol] | 0 | [symbol] | 0.999 | 2267 |
| [symbol] _UNKNOWN | 182 | [symbol] | [symbol] | 0 | [symbol] | 0.399 | 1452 |
| [symbol] | 95 | [symbol] | [symbol] | 0 | [symbol] | 0.35 | 1640 |
| [symbol] _CDH1_TARGETS_1_UP | 85 | [symbol] | [symbol] | 0 | [symbol] | 0.999 | 1592 |
| [symbol] _AGING_KIDNEY_NO_BLOOD_UP | 106 | [symbol] | [symbol] | 0 | [symbol] | 0.999 | 902 |
| MEISSNER_NPS_HCP_WITH_ [symbol] | 218 | [symbol] | [symbol] | 0 | [symbol] | 0.999 | 1487 |
| [symbol] _EWING_SARCOMA_PROGENITOR_UP | 204 | [symbol] | [symbol] | 0 | [symbol] | 0.999 | 1688 |
| REACTOME_ [symbol] _LIGAND_BINDING | 77 | [symbol] | [symbol] | 0 | [symbol] | 0.999 | [symbol] |
| [symbol] | 135 | [symbol] | [symbol] | 0 | [symbol] | 0.47 | 1390 |
| [symbol] _WITH_H3_UNMETHYLATED | 178 | [symbol] | [symbol] | 0 | [symbol] | 0.999 | 891 |
| GAUSSMANN_ [symbol] FUSION_TARGETS_ [symbol] | 107 | [symbol] | [symbol] | 0 | [symbol] | 0.999 | 1171 |
| MEISSNER_BRAIN_HCP_WITH_ [symbol] _ | 494 | [symbol] | [symbol] | 0 | [symbol] | 0.999 | 1587 |
| AND_ [symbol] | | | | | | | |
| WONG_ADULT_TISSUE_STEM_MODULE | 373 | [symbol] | [symbol] | 0 | [symbol] | 0.999 | 1755 |
| [symbol] | [symbol] | [symbol] | [symbol] | 0 | [symbol] | 0.522 | 1807 |
| [symbol] | 97 | [symbol] | [symbol] | 0 | [symbol] | 0.106 | 1817 |
| WANG_ [symbol] _TARGETS | [symbol] | [symbol] | [symbol] | 0 | [symbol] | 0.999 | 1585 |
| [symbol] | 140 | [symbol] | [symbol] | 0 | [symbol] | 0.523 | 1321 |
| [symbol] | 212 | [symbol] | [symbol] | 0 | [symbol] | 0.999 | 1298 |
| MIKKELSEN_NPC_ICP_WIHT_ [symbol] | 208 | [symbol] | [symbol] | 0 | [symbol] | 1 | 1286 |
| [symbol] | 123 | [symbol] | [symbol] | 0 | [symbol] | 0.674 | [symbol] |
| [symbol] | 119 | [symbol] | [symbol] | 0 | [symbol] | 0.672 | 1313 |
| [symbol] _UNKNOWN | 333 | [symbol] | [symbol] | 0 | [symbol] | 0.579 | 1777 |
| [symbol] | 113 | [symbol] | [symbol] | 0 | [symbol] | 0.603 | 1599 |
| [symbol] | 105 | [symbol] | [symbol] | 0 | [symbol] | 0.656 | 1271 |
| [symbol] _EXTRADIOL_RESPONSE_24HR_DN | 309 | −0.27060787 | −1.650879 | 0 | 0.04567584 | 1 | 1663 |
| GATA1_ [symbol] | 100 | −0.3178839 | −0.6226 | 0 | 0.0720975 | 0.755 | 1581 |
| [symbol] | 127 | [symbol] | −0.158209 | 0 | 0.04985837 | 0.901 | 1271 |
| [symbol] _TAMOXIFEN_RESISTANCE_DN | 137 | −0.3024071 | [symbol] | 0 | 0.0501198 | 1 | 1304 |
| [symbol] | 105 | −0.3122923 | −1.604354 | 0 | [symbol] | 0.806 | 1834 |
| [symbol] _UNKNOWN | 440 | [symbol] | [symbol] | 0 | [symbol] | 0.898 | 1899 |
| ECEVEDO_LIVER_CANCER_WITH_ [symbol] | 94 | [symbol] | −1.63057 | 0 | 0.05167929 | 1 | 1220 |
| [symbol] | 111 | [symbol] | [symbol] | 0 | [symbol] | 0.895 | 1439 |
| [symbol] | 103 | [symbol] | [symbol] | 0 | [symbol] | 0.893 | 1132 |
| MANALO_HYPOXIA_UP | 125 | [symbol] | [symbol] | 0 | [symbol] | 1 | 1438 |
| [symbol] | 101 | [symbol] | [symbol] | 0 | [symbol] | 0.865 | 1889 |
| [symbol] | 105 | [symbol] | [symbol] | 0 | [symbol] | 0.845 | 1777 |
| DAZARD_RESPONSE_ [symbol] | 133 | [symbol] | [symbol] | 0 | [symbol] | 1 | 1867 |
| [symbol] | 106 | [symbol] | [symbol] | 0 | 0.06134814 | 0.943 | 1314 |
| [symbol] | 361 | [symbol] | −1.491485 | 0 | [symbol] | 0.984 | 1267 |
| [symbol] _ONCOGENIC_SIGNATURE | 149 | [symbol] | [symbol] | 0 | [symbol] | 1 | 1342 |
| CHICAS_ [symbol] _TARGETS_ [symbol] | 324 | [symbol] | [symbol] | 0 | [symbol] | 1 | 881 |
| [symbol] | 108 | [symbol] | −1.47767 | 0 | [symbol] | 0.993 | 1486 |
| [symbol] _RESPONSE_TO_ARSENITE | 141 | −0.2875814 | −1.549565 | 0 | [symbol] | 1 | 1633 |
| [symbol] _UV_RESPONSE_KERATINOCYTE_UP | 283 | [symbol] | −1.551263 | 0 | [symbol] | 1 | 1498 |
| [symbol] _RESPONSE_TO_ [symbol] _UP | 236 | [symbol] | [symbol] | 0 | [symbol] | 1 | 1621 |
| MIKKELSEN_ES_ [symbol] _WITH_ [symbol] | 288 | [symbol] | [symbol] | 0 | [symbol] | 1 | 1557 |
| [symbol] _TARGETS_DN | 370 | [symbol] | [symbol] | 0 | 0.08460882 | 1 | 1829 |
| [symbol] _UNKNOWN | 490 | −0.226161 | −1.410285 | 0 | [symbol] | 0.999 | 1843 |
| [symbol] _UNKNOWN | 168 | [symbol] | [symbol] | 0 | [symbol] | 0.999 | 1777 |
| [symbol] _HEART_ATRIUM_VS_VENTRICLE_UP | 144 | [symbol] | [symbol] | 0 | [symbol] | 1 | 1710 |
| [symbol] _AGING_BRAIN_UP | 172 | [symbol] | [symbol] | 0 | [symbol] | 1 | 1575 |
| [symbol] _TAGETS | 129 | [symbol] | [symbol] | 0 | [symbol] | 1 | 2050 |
| [symbol] _PROSTATE_CANCER_DN | 242 | [symbol] | [symbol] | 0 | [symbol] | 1 | 1602 |
| [symbol] | 155 | [symbol] | [symbol] | 0 | [symbol] | 1 | 1297 |
| MARTENS_TRETINOIN_RESPONSE_DN | 401 | [symbol] | [symbol] | 0 | [symbol] | 1 | 962 |
| [symbol] _ENDOCRINE_THERAPY_RESISTANCE_1 | 297 | [symbol] | [symbol] | 0 | [symbol] | 1 | 1256 |
| RODRIGUES_THYROID_CARCINOMA_POORLY_ | 454 | [symbol] | [symbol] | 0 | 0.13657138 | 1 | 1642 |
| DIFFERENTIATED_DN | | | | | | | |
| KIM_BIPOLAR_DISORDER_OLIGODENDROCYTE_ | 411 | −0.221016 | [symbol] | 0 | [symbol] | 1 | 1167 |
| DENSITY_CORR_UP | | | | | | | |

[symbol]

indicates data missing or illegible when filed

TABLE 3
FR Responsive Downregulated Genes

| | | | | |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

indicates data missing or illegible when filed

TABLE 4
FR responsive Downregulated Genes GO

| | | # | | | |
|---|---|---|---|---|---|
| | # Genes | Genes in | | | |
| | in Gene | overlap | | | FDR |
| Gene Set Name | Set (K) | (k) | [symbol] | p-value | q-value |
| GO_NEUROGENESIS | 1402 | 92 | 0.0656 | 1.13E−33 | 5.97E−30 |
| GO_CELL_DEVELOPMENT | 1425 | 91 | 0.0638 | 2.19E−32 | 6.76E−29 |
| GO_NEURON_DIFFERENTIATION | 874 | 70 | 0.0801 | 70.9E−31 | 1.46E−27 |
| GO_REGULATION_OF_ [symbol] _DEVELOPMENT | 1672 | 95 | 0.0568 | 6.71E−30 | 1.04E−26 |
| GO_TISSUE_DEVELOPMENT | 1518 | 90 | 0.0595 | 1.15E−29 | 1.42E−26 |
| GO_EMBRYO_DEVELOPMENT | 894 | 59 | 0.0772 | 1.79E−29 | 1.84E−26 |
| GO_REGULATION_OF_CELL_DIFFERENTIATION | 1402 | 83 | 0.0556 | 1.58E−25 | 1.33E−22 |
| GO_ORGAN_MORPHOGENESIS | 841 | 62 | 0.0737 | 1.75E−25 | 1.33E−22 |
| GO_EMBRYONIC_MORPHOGENESIS | 939 | 50 | 0.0928 | 3.90E−25 | 2.67E−22 |
| CO_REGULATION_OF_CELL_PROLIFERATION | 1486 | 82 | 0.0548 | 8.29E−25 | 5.11E−22 |
| GO_TUBE_DEVELOPMENT | 552 | 50 | 0.0006 | 1.14E−24 | 6.40E−22 |
| GO_EPITHELIUM_DEVELOPMENT | 945 | 64 | 0.0677 | 2.76E−24 | 1.42E−23 |
| GO_POSITIVE_REGULATION_OF_CELL_COMMUNICATION | 1532 | 80 | 0.0522 | 6.78E−23 | 3.21E−20 |
| GO_TISSUE_MORPHOGENESIS | 533 | 47 | 0.0887 | 9.19E−23 | 4.05E−20 |
| GO_UROGENITAL_SYSTEM_DEVELOPMENT | 299 | 36 | 0.1204 | 3.28E−22 | 1.28E−19 |
| GO_INTRACELLULAR_ [symbol] _TRANSDUCTION | 1572 | 80 | 0.0509 | 3.33E−22 | 1.28E−19 |
| GO_NUCLEIC_ACID_BINDING_TRANSCRIPTION_FACTOR_ACTIVITY | 1199 | 29 | 0.0575 | 4.00E−22 | 1.45E−19 |
| GO_ANATOMICAL_STRUCTURE_FORMATION_INVOLVED_IN_MORPHOGENESIS | 957 | 61 | 0.0637 | 7.51E−22 | 2.57E−19 |
| GO_CENTRAL_NERVOUS_SYSTEM_DEVELOPMENT | 872 | 58 | 0.0665 | 1.04E−23 | 3.37E−19 |
| GO_MORPHOGENESIS_OF_AN_EPITHELIUM | 400 | 40 | 0.1000 | 1.59E−21 | 4.89E−19 |
| G0_CELL_MORPHOGENESIS_INVOLVED_IN_DIFFERENTIATION | 513 | 44 | 0.855 | 6.52E−21 | 1.92E−18 |
| GO_POSITIVE_REGULATION_OF_MULTICELLULAR_ORGANISMAL_PROCESS | 1395 | 72 | 0.0516 | 2.20E−20 | 6.17E−18 |
| GO_POSITIVE_REGULATION_OF_BIOSYNTHETIC_PROCESS | 2805 | 83 | 0.0460 | 2.59E−20 | 7.65E−18 |
| GO_REGULATION_OF_INTRACELLULAR_SIGNAL_TRANSDUCTION | 1656 | 79 | 0.0477 | 2.98E−20 | 7.65E−18 |
| GO_POSITIVE_REGULATION_OF_GENE_DEXPRESSION | 1733 | 81 | 0.0467 | 3.25E−20 | 8.01E−18 |
| GO_CARDIOVASCULAR_SYSTEM_DEVELOPMENT | 788 | 53 | 0.0673 | 3.87E−20 | 8.84E−18 |
| GO_CICULATORY_SYSTEM_DEVELOPMENT | 788 | 53 | 0.0673 | 3.87E−20 | 8.84E−18 |
| GO_MOVEMENT_OF_CELL_OF_SUBCELLULAR_COMPONENT | 1275 | 68 | 0.0533 | 4.55E−20 | 1.05E−17 |
| GO_RESPONSE_TO_EXTERNAL_STIMULUS | 1823 | 83 | 0.0456 | 4.95E−20 | 1.05E−17 |
| GO_NEURON_PROJECTION_DEVELOPMENT | 545 | 44 | 0.0807 | 6.81E−20 | 1.40E−17 |
| GO_INTRINSIC_COMPONENT_OF_PLASMA_MEMBRANE | 1649 | 78 | 0.0475 | 8.67E−20 | 1.68E−17 |
| GO_REGULATION_OF_ANATOMICAL_STRUCTURE_MORPHOGENESIS | 1021 | 60 | 0.0588 | 8.73E−20 | 1.68E−17 |
| GO_NEURON_DEVELOPMENT | 687 | 49 | 0.0713 | 9.44E−20 | 1.76E−17 |
| DO_REGULATION_OF_TRANSPORT | 1804 | 82 | 0.0455 | 1.00E−19 | 1.82E−17 |
| GO_REGULATION_OF_NERVOUS_SYSTEM_DEVELOPMENT | 750 | 51 | 0.0680 | 1.27E−19 | 2.24E−17 |
| GO_POSITIVE_REGULATION_OF_RESPONSE_TO_STIMULUS | 1929 | 85 | 0.0441 | 1.36E−19 | 2.33E−17 |
| GO_CELL_PROJECTION | 1785 | 81 | 0.0454 | 1.96E−19 | 3.27E−17 |
| GO_TUBE_MORPHOGENESIS | 323 | 34 | 0.1053 | 3.48E−19 | 5.64E−17 |
| GO_CELL_PROJECTION_ORGANIZATION | 902 | 55 | 0.0610 | 6.25E−19 | 9.89E−17 |
| GO_EMBRYONIC_ORGAN_DEVELOPMENT | 405 | 37 | 0.0911 | 1.19E−18 | 1.83E−16 |
| GO_RECEPTION_BINDING | 1476 | 71 | 0.0481 | 1.84E−18 | 2.77E−16 |
| GO_NEURON_PART | 1265 | 55 | 0.0514 | 2.21E−18 | 3.24E−16 |
| GO_CELLULAR_COMPONENT_MORPHOGENESIS | 900 | 54 | 0.600 | 2.58E−18 | 3.84E−16 |
| GO_POSITIVE_REGULATION_OF_MOLECULAR_FUNCTION | 1791 | 79 | 0.0441 | 2.83E−18 | 3.97E−16 |
| GO_APPENDAGE_DEVELOPMENT | 169 | 25 | 0.1479 | 5.36E−18 | 7.19E−16 |
| GO_LIMB_DEVELOPMENT | 169 | 25 | 0.1479 | 5.36E−18 | 7.19E−16 |
| GO_EXTRACELLULAR_MATRIX | 425 | 37 | 0.0869 | 5.83E−18 | 7.65E−16 |
| GO_NEURON_PROJECTION_MORPHOGENESIS | 402 | 36 | 0.0896 | 6.17E−18 | 7.92E−16 |
| GO_REPRODUCTIVE | 1297 | 65 | 0.0503 | 7.52E−18 | 9.51E−16 |
| GO_SKELETAL_SYSTEM_DEVELOPMENT | 455 | 38 | 0.0835 | 7.71E−18 | 9.51E−16 |

[symbol]

indicates data missing or illegible when filed

TABLE 5
FR Responsive Downregulated Genes GSEA

| | | | | | |
|---|---|---|---|---|---|
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |

[symbol]

indicates data missing or illegible when filed

### Example 6: Determine the Therapeutic Benefit in Mouse Models

In this example we will determine whether FR has potential to provide vision sparing therapy for primary (ocular) UM tumors. Treating primary UM tumors via intravitreal rather than systemic administration of FR might provide a more effective and better tolerated means of intervening pharmacologically, because this approach might enable FR to be administered at higher dose. Based on our preliminary studies of UM cells in culture, intravitreal injection might be therapeutically beneficial by inhibiting tumor growth, promoting apoptosis, and/or pushing tumor re-differentiation toward melanocytic phenotypes less likely to metastasize. FR might provide vision-sparing therapy because our preliminary data indicate that it preserves retina structure. Our second priority is to determine whether FR administered systemically could provide therapeutic benefit for metastatic UM tumors. An important goal in both priorities will be to determine whether the effects of FR in models of primary or metastatic UM tumors require reinstatement of PRC2-mediated gene silencing, thereby testing the pathological and therapeutic relevance of the novel signaling mechanism suggested by the studies of human UM cells in culture.

No mouse or other animal model of UM fully recapitulates the disease; instead, various models are used based on the specific questions to be answered. Here we will use two well-accepted mouse models: a transgenic model of primary UM tumors driven by conditional expression of constitutively active Gαq (Q209L) in melanocytes, and an orthotopic transplant model using human UM cell lines and immune deficient (NSG) mice. Also contemplated are PDX models of UM because their tumor cells might better mimic response of human UM tumors to FR.

ERG recordings of intact mice will be used to determine what concentration of intravitreally injected FR affects retina function. One eye will be injected with FR and the other with vehicle as a control. Intravitreal injection itself does not affect retina function as assessed by ERGs. Standard corneal ERGs will be recorded 24 h after injection using scotopic and photopic illumination to identify effects on rod and/or cones, bipolar neurons, or feedback between retinal neurons. ERGs will be recorded weekly thereafter to determine how long the effects of a single FR injection persist. If ERGs do not return to normal, retina histology will be used to assess whether structural defects indicative of cell death have occurred. Although substantial, sustained impairment of retina function would suggest that FR is unlikely to provide vision-sparing therapy of primary UM tumors, this inhibitor could still be therapeutically beneficial if it impairs tumor growth and/or development of metastatic disease.

Next we will determine whether FR targets primary ocular tumors in a transgenic mouse model of UM. These experiments will use the only established transgenic model of UM in which conditional expression of constitutively active Gαq(Q209L) is induced by Mitf-CRE in uveal and other melaoncytes. This model has key advantages over transplant models. First, it is highly penetrant and reproducible. Occular tumors occur in 100% of mice within 4 weeks of age. Second, hematogenous dissemination of tumor cells to the lung occurs in >90% of mice by 3 months of age, making this transgenic model well suited for determining whether FR inhibits metastatic dissemination or growth. Although the transgenic models do not recapitulate other genetic changes characteristic of human UM, it is the only mouse model that produces primary tumors within the same compartment (uveal tract) as in human UM. In contrast, tumor xenografts develop intravitreally are inappropriate for indicating how tumors arising within the uveal tract might respond to FR.

Transgenic uveal melanoma tumors will be imaged non-invasively, by spectral-domain optical coherence tomography (SD-OCT) and angiography. These techniques enable ocular tumor volume and blood supply recruitment to be followed over time. Baseline images will be acquired in 4-week old mice when tumors are small but evident. The next day, vehicle will be injected intravitreally into one eye and FR at various concentrations, as suggested by studies of FR on retina function, into the other eye. The same imaging and intravitreal injection procedures will be repeated one week later; further injections will not be performed because they would damage the eye. Final tumor images will be acquired in 6-week old mice (i.e. one week after, the 2nd FR or vehicle injection), by which time control tumors have expanded to occupy −30% of the intravitreal space. Inhibition of tumor growth as a function of FR concentration will be determined. Tumors will be harvested and analyzed by immunoblotting or immunohistochemistry for the activity state of signaling pathways downstream of constitutively active Gαq/11 in UM (YAP; MEK), to determine dose of FR is sufficient to inhibit constitutively active Gαq signaling in tumors.

Next we will determine whether treatment reinstates PRC2-mediated repression in primary ocular tumors and whether this mechanism is required for FR to inhibit tumor growth. RNA-Seq will be performed to determine whether intraocular injection of FR in the transgenic model of UM reinstates PRC2-mediated repression. Second, we will determine whether maintaining PRC2 in an inactive state with an Ezh1/2 inhibitor (GSK503) interferes with the ability of FR to blunt tumor growth and/or reinstate PRC2-mediated silencing. Using the same schedule involving two weekly intravitreal injections described in above, vehicle or FR at its effective concentration will be co-injected with GSK503 at a concentration known to inactivate the catalytic subunits of PRC2 complexes in vivo. Tumor growth will be imaged by SD-OCT. PRC2 inhibition in ocular tumors will be detected by immunoblotting for histone H3K27me3 normalized to total histone H3. The effects of FR injected with or without GSK503 on expression of PRC2 target genes will be determined by RNA-Seq. Based on our studies of human UM cells, it is expected that FR will reinstate silencing of PRC2-regulated genes in transgenic UM tumors, whereas this effect of FR will be blocked or blunted by inhibition of Ezh1/2.

Determine whether FR inhibits development of lung metastases in the transgenic UM model. This question will be addressed in two ways, both of which will examine the appearance of pigmented lung lesions in 3-month old transgenic mice, which normally occur with high penetrance by this time point. We will determine whether FR administered systemically by daily subcutaneous injection (0.1-0.3 mg/kg) affects the growth of primary ocular tumors and/or lung lesions the transgenic model.

### REFERENCES

1. A. G. Gilman, G proteins: transducers of receptor-generated signals. Annu. Rev. Biochem. 56, 615-49 (1987).
2. M. O'Hayre, M. S. Degese, J. S. Gutkind, Novel insights into G protein and G protein-coupled receptor signaling in cancer. Curr. Opin. Cell Biol. 27, 126-35 (2014).
3. M. D. Shirley, H. Tang, C. J. Gallione, J. D. Baugher, L. P. Frelin, B. Cohen, P. E. North, D. A. Marchuk, A. M. Comi, J. Pevsner, Sturge-Weber syndrome and port-wine stains caused by somatic mutation in GNAQ. N Engl J Med. 368, 1971-1979 (2013).
4. M. D. Onken, L. A. Worley, M. D. Long, S. Duan, M. L. Council, A. M. Bowcock, J. W. Harbour, Oncogenic mutations in GNAQ occur early in uveal melanoma. Invest Ophthalmol Vis Sci. 49, 5230-5234 (2008).
5. C. D. Van Raamsdonk, V. Bezrookove, G. Green, J. Bauer, L. Gaugler, J. M O'Brien, E. M. Simpson, G. S. Barsh, B. C. Bastian, Frequent somatic mutations of GNAQ in uveal melanoma and blue naevi. Nature. 457, 599-602 (2009).
6. C. D. Van Raamsdonk, K. G. Griewank, M. B. Crosby, M. C. Garrido, S. Vemula, T. Wiesner, A. C. Obenauf, W. Wackernagel, G. Green, N. Bouvier, M. M. Sozen, G. Baimukanova, R. Roy, A. Heguy, I. Dolgalev, R. Khanin, K. Busam, M. R. Speicher, J. O'Brien, B. C. Bastian, Mutations in GNA11 in Uveal Melanoma. N Engl J Med (2010).
7. Y. Yonekawa, I. K. Kim, Epidemiology and management of uveal melanoma. Hematol Oncol Clin North Am. 26, 1169-1184 (2012).
8. C. M. Balch, J. E. Gershenwald, S. J. Soong, J. F. Thompson, M. B. Atkins, D. R. Byrd, A. C. Buzaid, A. J. Cochran, D. G. Coit, S. Ding, A. M. Eggermont, K. T. Flaherty, P. A. Gimotty, J. M. Kirkwood, K. M. McMasters, M. C. J. Mihm, D. L. Morton, M. I. Ross, A. J. Sober, V. K. Sondak, Final version of 2009 AJCC melanoma staging and classification. J Clin Oncol. 27, 6199-6206 (2009).
9. R. D. Carvajal, G. K. Schwartz, T. Tezel, B. Marr, J. H. Francis, P. D. Nathan, Metastatic disease from uveal melanoma: treatment options and future prospects. Br. J. Ophthalmol. 101, 38-44 (2017).
10. A. V Smrcka, Molecular targeting of Gα and Gβγ subunits: a potential approach for cancer therapeutics. Trends Pharmacol. Sci. 34, 290-8 (2013).
11. J. M. L. Ostrem, K. M. Shokat, Direct small-molecule inhibitors of KRAS: from structural insights to mechanism-based design. Nat. Rev. Drug Discov. 15, 771-785 (2016).
12. V. Chua, D. Lapadula, C. Randolph, J. L. Benovic, P. Wedegaertner, A. E. Aplin, Dysregulated GPCR Signaling and Therapeutic Options in Uveal Melanoma. Mol Cancer Res (2017).
13. P. Chidiac, V. S. Markin, E. M. Ross, Kinetic control of guanine nucleotide binding to soluble Galpha(q). Biochem. Pharmacol. 58, 39-48 (1999).
14. J. L. Blank, A. H. Ross, J. H. Exton, Purification and characterization of two G-proteins that activate the beta 1 isozyme of phosphoinositide-specific phospholipase C. Identification as members of the Gq class. J. Biol. Chem. 266, 18206-16 (1991).
15. G. Berstein, J. L. Blank, A. V Smrcka, T. Higashijima, P. C. Sternweis, J. H. Exton, E. M. Ross, Reconstitution of agonist-stimulated phosphatidylinositol 4,5 bisphosphate hydrolysis using purified ml muscarinic receptor, Gq/11, and phospholipase C-beta 1. J. Biol. Chem. 267, 8081-8 (1992).
16. G. G. Tall, A. M. Krumins, A. G. Gilman, Mammalian Ric-8A (synembryn) is a heterotrimeric Galpha protein guanine nucleotide exchange factor. J. Biol. Chem. 278, 8356-62 (2003).
17. P. Chan, C. J. Thomas, S. R. Sprang, G. G. Tall, Molecular chaperoning function of Ric-8 is to fold nascent heterotrimeric G protein a subunits. Proc. Natl. Acad. Sci. U.S.A. 110, 3794-9 (2013).
18. T. W. Traut, Physiological concentrations of purines and pyrimidines. Mol. Cell. Biochem. 140, 1-22 (1994).
19. A. Nishimura, K. Kitano, J. Takasaki, M. Taniguchi, N. Mizuno, K. Tago, T. Hakoshima, H. Itoh, Structural basis for the specific inhibition of heterotrimeric Gq protein by a small molecule. Proc Natl Acad Sci USA. 107, 13666-13671 (2010).
20. R. Schrage, A. L. Schmitz, E. Gaffal, S. Annala, S. Kehraus, D. Wenzel, K. M. Büllesbach, T. Bald, A. Inoue, Y. Shinjo, S. Galandrin, N. Shridhar, M. Hesse, M. Grundmann, N. Merten, T. H. Charpentier, M. Martz, A. J. Butcher, T. Slodczyk, S. Armando, M. Effern, Y. Namkung, L. Jenkins, V. Horn, A. Stößel, H. Dargatz, D. Tietze, D. Imhof, C. Galés, C. Drewke, C. E. Müller, M. Hölzel, G. Milligan, A. B. Tobin, J. Gomeza, H. G. Dohlman, J. Sondek, T. K. Harden, M. Bouvier, S. A. Laporte, J. Aoki, B. K. Fleischmann, K. Mohr, G. M. König, T. Tüting, E. Kostenis, The experimental power of FR900359 to study Gq-regulated biological processes. Nat Commun. 6, 10156 (2015).
21. J. Takasaki, T. Saito, M. Taniguchi, T. Kawasaki, Y. Moritani, K. Hayashi, M. Kobori, A novel Galphaq/11-selective inhibitor. J Biol Chem. 279, 47438-47445 (2004).
22. S. P. Heximer, N. Watson, M. E. Linder, K. J. Blumer, J. R. Hepler, RGS2/G0S8 is a selective inhibitor of Gqalpha function. Proc. Natl. Acad. Sci. U.S.A 94, 14389-93 (1997).
23. V. Villalobos, S. Naik, M. Bruinsma, R. S. Dothager, M. H. Pan, M. Samrakandi, B. Moss, A. Elhammali, D. Piwnica-Worms, Dual-color click beetle luciferase heteroprotein fragment complementation assays. Chem Biol. 17, 1018-1029 (2010).
24. S. L. Scherer, M. D. Cain, S. M. Kanai, K. M. Kaltenbronn, K. J. Blumer, Regulation of neurite morphogenesis by interaction between R7 regulator of G protein signaling complexes and G protein subunit Gα13. J. Biol. Chem. 292, 9906-9918 (2017).
25. T. Flock, C. N. J. Ravarani, D. Sun, A. J. Venkatakrishnan, M. Kayikci, C. G. Tate, D. B. Veprintsev, M. M. Babu, Universal allosteric mechanism for Gα activation by GPCRs. Nature. 524, 173-179 (2015).
26. F.-X. Yu, J. Luo, J.-S. Mo, G. Liu, Y. C. Kim, Z. Meng, L. Zhao, G. Peyman, H. Ouyang, W. Jiang, J. Zhao, X. Chen, L. Zhang, C.-Y. Wang, B. C. Bastian, K. Zhang, K.-L. Guan, Mutant Gq/11 Promote Uveal Melanoma Tumorigenesis by Activating YAP. Cancer Cell. 25, 822-830 (2014).
27. X. Feng, M. S. Degese, R. Iglesias-Bartolome, J. P. Vague, A. A. Molinolo, M. Rodrigues, M. R. Zaidi, B. R. Ksander, G. Merlino, A. Sodhi, Q. Chen, J. S. Gutkind, Hippo-Independent Activation of YAP by the GNAQ Uveal Melanoma Oncogene through a Trio-Regulated Rho GTPase Signaling Circuitry. Cancer Cell. 25, 831-845 (2014).
28. T. G. Cotter, Apoptosis and cancer: the genesis of a research field. Nat. Rev. Cancer. 9, 501-507 (2009).
29. K. W. Yip, J. C. Reed, Bcl-2 family proteins and cancer. Oncogene. 27, 6398-6406 (2008).
30. Y. H. Lim, A. Bacchiocchi, J. Qiu, R. Straub, A. Bruckner, L. Bercovitch, D. Narayan, J. McNiff, C. Ko, L. Robinson-Bostom, R. Antaya, R. Halaban, K. A. Choate, K. A. Choate, GNA14 Somatic Mutation Causes Congenital and Sporadic Vascular Tumors by MAPK Activation. Am. J. Hum. Genet. 99, 443-450 (2016).
31. M. D. Onken, J. P. Ehlers, L. A. Worley, J. Makita, Y. Yokota, J. W. Harbour, Functional gene expression analysis uncovers phenotypic switch in aggressive uveal melanomas. Cancer Res. 66, 4602-4609 (2006).
32. K. A. Matatall, O. A. Agapova, M. D. Onken, L. A. Worley, A. M. Bowcock, J. W. Harbour, BAP1 deficiency causes loss of melanocytic cell identity in uveal melanoma. BMC Cancer. 13, 371 (2013).
33. M. D. Onken, L. A. Worley, J. P. Ehlers, J. W. Harbour, Gene expression profiling in uveal melanoma reveals two molecular classes and predicts metastatic death. Cancer Res. 64, 7205-7209 (2004).
34. T. S. Mikkelsen, J. Hanna, X. Zhang, M. Ku, M. Wernig, P. Schorderet, B. E. Bernstein, R. Jaenisch, E. S. Lander, A. Meissner, Dissecting direct reprogramming through integrative genomic analysis. Nature. 454, 49-55 (2008).
35. A. Meissner, T. S. Mikkelsen, H. Gu, M. Wernig, J. Hanna, A. Sivachenko, X. Zhang, B. E. Bernstein, C. Nusbaum, D. B. Jaffe, A. Gnirke, R. Jaenisch, E. S. Lander, Genome-scale DNA methylation maps of pluripotent and differentiated cells. Nature. 454, 766-70 (2008).
36. I. Ben-Porath, M. W. Thomson, V. J. Carey, R. Ge, G. W. Bell, A. Regev, R. A. Weinberg, An embryonic stem cell-like gene expression signature in poorly differentiated aggressive human tumors. Nat. Genet. 40, 499-507 (2008).
37. B. Kaabi, G. Belaaloui, W. Benbrahim, K. Hamizi, M. Sadelaoud, W. Toumi, H. Bounecer, ADRA2A Germline Gene Polymorphism is Associated to the Severity, but not to the Risk, of Breast Cancer. Pathol. Oncol. Res. 22, 357-365 (2016).
38. M. Minoux, S. Holwerda, A. Vitobello, T. Kitazawa, H. Kohler, M. B. Stadler, F. M. Rijli, Science, in press, doi:10.1126/science.aa12913.
39. S. Landreville, O. A. Agapova, K. A. Matatall, Z. T. Kneass, M. D. Onken, R. S. Lee, A. M. Bowcock, J. W. Harbour, Histone deacetylase inhibitors induce growth arrest and differentiation in uveal melanoma. Clin Cancer Res. 18, 408-416 (2012).
40. T. E. Hughes, H. Zhang, D. E. Logothetis, C. H. Berlot, Visualization of a Functional Gα q-Green Fluorescent Protein Fusion in Living Cells. J. Biol. Chem. 276, 4227-4235 (2001).
41. M. A. Booden, D. P. Siderovski, C. J. Der, Leukemia-associated Rho guanine nucleotide exchange factor promotes G alpha q-coupled activation of RhoA. Mol. Cell. Biol. 22, 4053-61 (2002).
42. M. Mohseni, J. Sun, A. Lau, S. Curtis, J. Goldsmith, V. L. Fox, C. Wei, M. Frazier, O. Samson, K.-K. Wong, C. Kim, F. D. Camargo, F. D. Camargo, A genetic screen identifies an LKB1-MARK signalling axis controlling the Hippo-YAP pathway. Nat. Cell Biol. 16, 108-117 (2013).
43. C. R. Evelyn, S. M. Wade, Q. Wang, M. Wu, J. A. Iniguez-Lluhi, S. D. Merajver, R. R. Neubig, CCG-1423: a small-molecule inhibitor of RhoA transcriptional signaling. Mol. Cancer Ther. 6, 2249-2260 (2007).
44. R. B. Delston, K. A. Matatall, Y. Sun, M. D. Onken, J. W. Harbour, p38 phosphorylates Rb on Ser567 by a novel, cell cycle-independent mechanism that triggers Rb-Hdm2 interaction and apoptosis. Oncogene. 30, 588-599 (2011).
45. A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles. Proc Natl Acad Sci USA. 102, 15545-15550 (2005).

1. A method of allosterically inhibiting nucleotide exchange of a constitutively active G protein α-subunit in a cell by trapping the G protein α-subunit in a GDP-bound state, method comprising:
contacting the cell with an effective amount of FR900359: 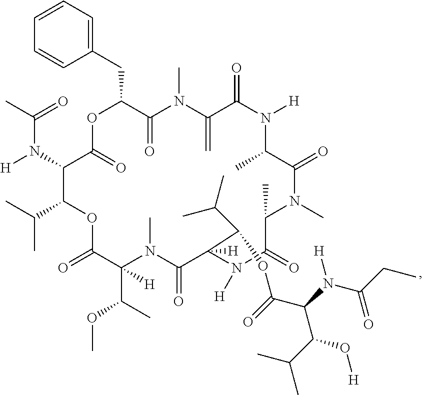 [SMILES: C=C1C(=O)N[C@@H](C)C(=O)N(C)[C@@H](C)C(=O)N[C@H]([C@@H](OC(=O)[C@@H](NC(=O)CC)[C@H](O)C(C)C)C(C)C)C(=O)N(C)[C@@H]([C@H](C)OC)C(=O)O[C@H](C(C)C)[C@H](NC(C)=O)C(=O)O[C@H](Cc2ccccc2)C(=O)N1C]
YM-254890: 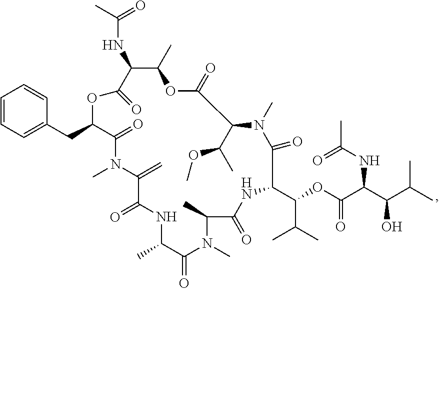
or a derivative thereof, wherein the effective amount allosterically inhibits GDP release from the G protein α-subunit.

2. The method of claim 1, wherein said GDP bound constitutively active Gα subunit assembles into Gαβγ heterotrimers, further suppressing GDP release and stabilizing the heterotrimer in an inactive state.

3. The method of claim 1, wherein contacting the cell occurs in vitro, in vivo or ex vivo.

4. The method of claim 1, wherein the cell is in a subject and the subject has or is suspected of having a disease or disorder associated with constitutively active G-protein signaling.

5. (canceled)

6. The method of claim 2, wherein stabilizing the Gαβγ heterotrimer in an inactive state reduces a downstream constituently active G-protein signaling pathway, wherein the downstream signaling pathways include Yes-associated protein (YAP) activity, adenylyl cyclase, phospholipase C, the mitogen activated protein kinases (MAPKs), extracellular signal regulated kinase (ERK) c-Jun-NH2-terminal kinase (JNK) or p38 MAPK.

7. The method of claim 4, wherein the disease or disorder associated with constitutively active G-protein signaling is selected from growth hormone-secreting pituitary tumors, tumors derived from Nevus of Ota, colon cancer, lung cancer, adenocarcinoma, skin melanoma, thyroid adenoma, cholera, and Sturge-Weber Syndrome.

8. (canceled)

9. A method of treating uveal melanoma in a subject in need thereof, method comprising:
administering to the subject a composition comprising an effective amount of FR900359: 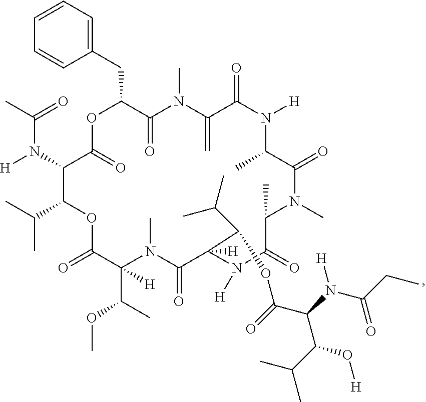 [SMILES: C=C1C(=O)N[C@@H](C)C(=O)N(C)[C@@H](C)C(=O)N[C@H]([C@@H](OC(=O)[C@@H](NC(=O)CC)[C@H](O)C(C)C)C(C)C)C(=O)N(C)[C@@H]([C@H](C)OC)C(=O)O[C@H](C(C)C)[C@H](NC(C)=O)C(=O)O[C@H](Cc2ccccc2)C(=O)N1C]
YM-254890: 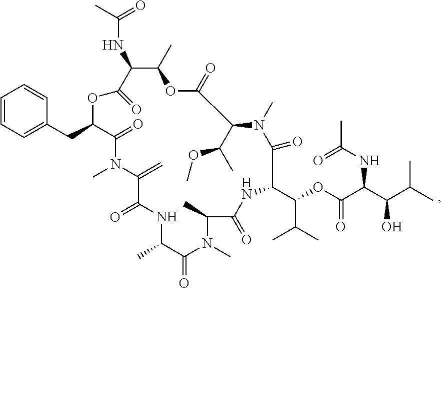
or a derivative thereof, wherein the effective amount allosterically inhibits GDP release from an constitutively active G protein α-subunit.

10. The method of claim 9, wherein the FR900359, YM-254890, or a derivative thereof down-regulates constitutively active G-protein signaling.

11. The method of claim 9, wherein uveal melanoma cell proliferation is reduced relatively to untreated uveal melanoma cells.

12. (canceled)

13. The method of claim 9, wherein 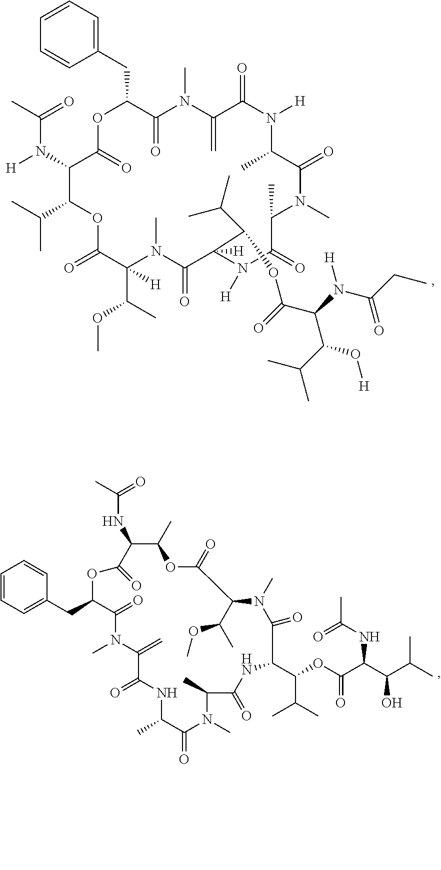
the uveal melanoma cells re-differentiate compared to untreated uveal melanoma cells.

14. The method of claim 13, wherein re-differentiation is determined by loss of spindle morphology, flatting of the cell, production of multiple projections, increased melanocytic pigmentation or combinations thereof.

15. The method of claim 13, wherein genes targeted by the polycomb repressive complex 2 (PRC2) are repressed.

16. The method of claim 15, wherein the repressed genes ADRA2A (alpha-adrenergic receptor-2A) and HAND2 (heart and neural crest derivatives expressed-2).

17. A method of treatment of a disease, disorder, or condition associated with constitutively active G protein signaling in a subject in need thereof, the method comprising:
administering to the subject a composition comprising a therapeutically effective amount of an composition comprising FR900359: 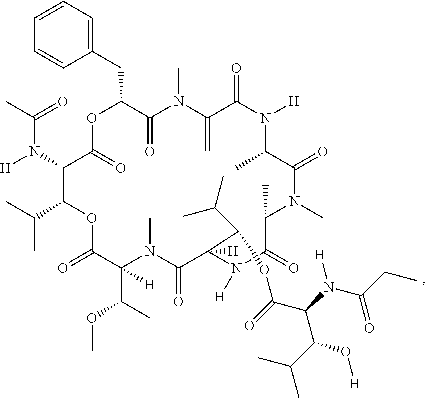 [SMILES: C=C1C(=O)N[C@@H](C)C(=O)N(C)[C@@H](C)C(=O)N[C@H]([C@@H](OC(=O)[C@@H](NC(=O)CC)[C@H](O)C(C)C)C(C)C)C(=O)N(C)[C@@H]([C@H](C)OC)C(=O)O[C@H](C(C)C)[C@H](NC(C)=O)C(=O)O[C@H](Cc2ccccc2)C(=O)N1C]
YM-254890: 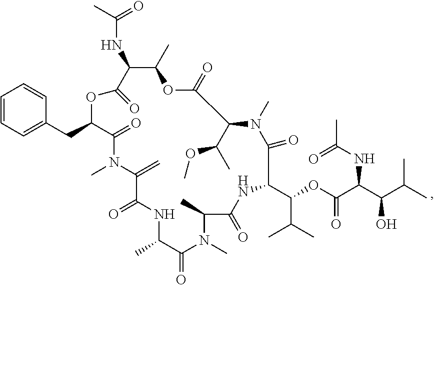
or a derivative thereof; wherein, the therapeutically effective amount reduces or prevents constitutively active G protein signaling.

18. The method of claim 17, wherein the disease, disorder, or condition is selected from growth hormone-secreting pituitary tumors, tumors derived from Nevus of Ota, constitutively active G protein mediated cancer (e.g. colon, lung, adenocarcinoma, skin melanoma, thyroid adenomas), cholera, and Sturge-Weber Syndrome

19.-20. (canceled)

21. The method of claim 17, wherein the effective amount allosterically inhibits GDP release from the G protein α-subunit.

22. The method of claim 17, wherein the disorder, or condition associated with constitutively active G protein signaling is a tumor or cancer and tumor or cancer cell growth is reduced relative to the untreated tumor or cancer cell.

23. The method of claim 17, wherein the disorder, or condition associated with constitutively active G protein signaling is a tumor or cancer and tumor or cancer metastasis is reduced relative to the untreated tumor or cancer cell.
